(19) United States
(12) Patent Application Publication
(10) Pub. No.: US 2023/0219921 A1
(43) Pub. Date: Jul. 13, 2023

(54) KCNT1 INHIBITORS AND METHODS OF USE

(71) Applicant: Praxis Precision Medicines, Inc., Boston, MA (US)

(72) Inventors: Andrew Mark GRIFFIN, L'lle Bizard (CA); Gabriel MARTINEZ BOTELLA, Wayland, MA (US)

(21) Appl. No.: 18/004,417

(22) PCT Filed: Jul. 6, 2021
(86) PCT No.: PCT/US2021/040486

§ 371 Date: Jan. 5, 2023

Related U.S. Application Data
(60) Provisional application No. 63/048,335, filed on Jul. 6, 2020.

(51) Int. Cl. C07D 333/38; C07D 409/12

(52) U.S. Cl. CPC C07D 333/38; C07D 409/12

(57) ABSTRACT

The present invention is directed to, in part, compounds and compositions useful for preventing and/or treating a neurological disease or disorder, a disease or condition relating to excessive neuronal excitability, and/or a gain-of-function mutation in a gene (e.g., KCNT1). Methods of treating a neurological disease or disorder, a disease or condition relating to excessive neuronal excitability, and/or a gain-of-function mutation in a gene such as KCNT1 are also provided herein.

### CROSS-REFERENCE TO RELATED APPLICATIONS

This application claims priority to and the benefit of U.S. Provisional Patent Application No. 63/048,335 filed Jul. 6, 2020, the contents of which are incorporated herein by reference in their entirety.

### BACKGROUND

KCNT1 encodes sodium-activated potassium channels known as Slack (Sequence like a calcium-activated K⁺ channel). These channels are found in neurons throughout the brain and can mediate a sodium-activated potassium current I_{KNa}. This delayed outward current can regulate neuronal excitability and the rate of adaption in response to maintained stimulation. Abnormal Slack activity have been associated with development of early onset epilepsies and intellectual impairment. Accordingly, pharmaceutical compounds that selectively regulate sodium-activated potassium channels, e.g., abnormal KCNT1, abnormal I_{KNa}, are useful in treating a neurological disease or disorder or a disease or condition related to excessive neuronal excitability and/or KCNT1 gain-of-function mutations.

### SUMMARY OF THE INVENTION

Described herein are compounds and compositions useful for preventing and/or treating a disease, disorder, or condition, e.g., a neurological disease or disorder, a disease, disorder, or condition associated with excessive neuronal excitability and/or a gain-of-function mutation in a gene, for example, KCNT1.

Thus, in one aspect, the present disclosure features a pharmaceutical composition comprising a compound of Formula (A):

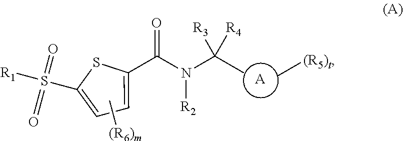

or a pharmaceutically acceptable salt thereof, wherein the variables are as defined herein, and a pharmaceutically acceptable excipient.

In another aspect, the present disclosure features a pharmaceutical composition comprising a compound of Formula (A-1), Formula (A-2), or Formula (A-3):

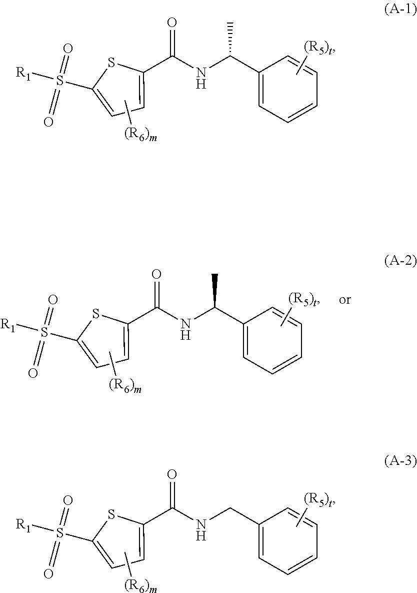

or a pharmaceutically acceptable salt thereof, wherein the variables are as defined herein, and a pharmaceutically acceptable excipient.

In another aspect, the present disclosure features a compound of Formula (II):

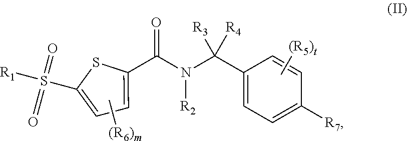

or a pharmaceutically acceptable salt thereof, wherein the variables are as defined herein.

In another aspect, the present disclosure features a compound of Formula (II-a):

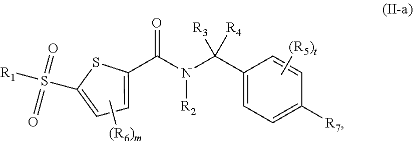

or a pharmaceutically acceptable salt thereof, wherein the variables are as defined herein.

In another aspect, the present disclosure features a compound of Formula (II-b):

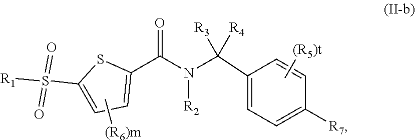

or a pharmaceutically acceptable salt thereof, wherein the variables are as defined herein.

In another aspect, the present disclosure features a compound of Formula (III):

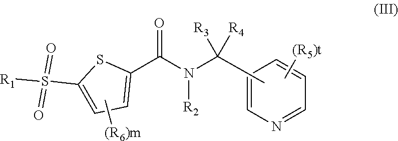

or a pharmaceutically acceptable salt thereof, wherein the variables are as defined herein.

In another aspect, the present disclosure provides a pharmaceutical composition comprising a compound disclosed herein (e.g., a compound of Formula (II), (II-a), (II-b), or (III), or a pharmaceutically acceptable salt thereof, and a pharmaceutically acceptable excipient.

In another aspect, the present disclosure provides a method of treating a neurological disease or disorder, wherein the method comprises administering to a subject in need thereof a compound disclosed herein (e.g., a compound of Formula (A), (A-1), (A-2), (A-3), (I), (I-a), (I-b), (II), (II-a), (II-b), or (III), or a pharmaceutically acceptable salt thereof) or a pharmaceutical composition disclosed herein (e.g., a pharmaceutical composition comprising a compound disclosed herein (e.g., a compound of Formula (A), (A-1), (A-2), (A-3), (I), (I-a), (I-b), (II), (II-a), (II-b), or (III), or a pharmaceutically acceptable salt thereof), and a pharmaceutically acceptable excipient).

In another aspect, the present disclosure provides a method of treating a disease or condition associated with excessive neuronal excitability, wherein the method comprises administering to a subject in need thereof a compound disclosed herein (e.g., a compound of Formula (A), (A-1), (A-2), (A-3), (I), (I-a), (I-b), (II), (II-a), (II-b), or (III), or a pharmaceutically acceptable salt thereof) or a pharmaceutical composition disclosed herein (e.g., a pharmaceutical composition comprising a compound disclosed herein (e.g., a compound of Formula (A), (A-1), (A-2), (A-3), (I), (I-a), (I-b), (II), (II-a), (II-b), or (III), or a pharmaceutically acceptable salt thereof), and a pharmaceutically acceptable excipient).

In another aspect, the present disclosure provides a method of treating a disease or condition associated with a gain-of-function mutation of a gene (e.g., KCNT1), wherein the method comprises administering to a subject in need thereof a compound disclosed herein (e.g., a compound of Formula (A), (A-1), (A-2), (A-3), (I), (I-a), (I-b), (II), (II-a), (II-b), or (III), or a pharmaceutically acceptable salt thereof) or a pharmaceutical composition disclosed herein (e.g., a pharmaceutical composition comprising a compound disclosed herein (e.g., a compound of Formula (A), (A-1), (A-2), (A-3), (I), (I-a), (I-b), (II), (II-a), (II-b), or (III), or a pharmaceutically acceptable salt thereof), and a pharmaceutically acceptable excipient).

In some embodiments, the neurological disease or disorder, the disease or condition associated with excessive neuronal excitability, or the disease or condition associated with a gain-of-function mutation of a gene (e.g., KCNT1) is epilepsy, an epilepsy syndrome, or an encephalopathy.

In some embodiments, the neurological disease or disorder, the disease or condition associated with excessive neuronal excitability, or the disease or condition associated with a gain-of-function mutation of a gene (e.g., KCNT1) is a genetic or pediatric epilepsy or a genetic or pediatric epilepsy syndrome.

In some embodiments, the neurological disease or disorder, the disease or condition associated with excessive neuronal excitability, or the disease or condition associated with a gain-of-function mutation of a gene (e.g., KCNT1) is a cardiac dysfunction.

In some embodiments, the neurological disease or disorder, the disease or condition associated with excessive neuronal excitability, or the disease or condition associated with a gain-of-function mutation of a gene (e.g., KCNT1) is selected from epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, cerebellar ataxia).

In some embodiments, the neurological disease or disorder, the disease or condition associated with excessive neuronal excitability, or the disease or condition associated with a gain-of-function mutation of a gene (e.g., KCNT1) is selected from the group consisting of cardiac arrhythmia, sudden unexpected death in epilepsy, Brugada syndrome, and myocardial infarction.

In some embodiments, the neurological disease or disorder, the disease or condition associated with excessive neuronal excitability, or the disease or condition associated with a gain-of-function mutation of a gene (e.g., KCNT1) is selected from pain and related conditions (e.g. neuropathic pain, acute/chronic pain, migraine, etc).

In some embodiments, the neurological disease or disorder, the disease or condition associated with excessive neuronal excitability, or the disease or condition associated with a gain-of-function mutation of a gene (e.g., KCNT1) is a muscle disorder (e.g. myotonia, neuromyotonia, cramp muscle spasms, spasticity).

In some embodiments, the neurological disease or disorder, the disease or condition associated with excessive neuronal excitability, or the disease or condition associated with a gain-of-function mutation of a gene (e.g., KCNT1) is selected from itch and pruritis, ataxia and cerebellar ataxias.

In some embodiments, the neurological disease or disorder, the disease or condition associated with excessive neuronal excitability, or the disease or condition associated with a gain-of-function mutation of a gene (e.g., KCNT1) is selected from psychiatric disorders (e.g. major depression, anxiety, bipolar disorder, schizophrenia).

In some embodiments, the neurological disease or disorder or the disease or condition associated with excessive neuronal excitability and/or a gain-of-function mutation in a gene (e.g., KCNT1) is selected from the group consisting of learning disorders, Fragile X, neuronal plasticity, and autism spectrum disorders.

In some embodiments, the neurological disease or disorder, the disease or condition associated with excessive neuronal excitability, or the disease or condition associated with a gain-of-function mutation of a gene (e.g., KCNT1) is selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

Other objects and advantages will become apparent to those skilled in the art from a consideration of the ensuing Detailed Description, Examples, and Claims.

### DETAILED DESCRIPTION OF THE INVENTION

As generally described herein, the present invention provides compounds and compositions useful for preventing and/or treating a disease, disorder, or condition described herein, e.g., a disease, disorder, or condition associated with excessive neuronal excitability, and/or a disease, disorder, or condition associated with gain-of-function mutations in KCNT1. Exemplary diseases, disorders, or conditions include epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, and Lennox Gastaut syndrome, seizures, leukodystrophy, leukoencephalopathy, intellectual disability, Multifocal Epilepsy, Generalized tonic clonic seizures, Drug resistant epilepsy, Temporal lobe epilepsy, cerebellar ataxia, Asymmetric Tonic Seizures) and cardiac dysfunctions (e.g., cardiac arrhythmia, Brugada syndrome, sudden unexpected death in epilepsy, myocardial infarction), pain and related conditions (e.g. neuropathic pain, acute/chronic pain, migraine, etc), muscle disorders (e.g. myotonia, neuromyotonia, cramp muscle spasms, spasticity), itch and pruritis, ataxia and cerebellar ataxias, psychiatric disorders (e.g. major depression, anxiety, bipolar disorder, schizophrenia), learning disorders, Fragile X, neuronal plasticity, and autism spectrum disorders.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E.L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; F may be in any isotopic form, including ¹⁸F and ¹⁹F; and the like.

The following terms are intended to have the meanings presented therewith below and are useful in understanding the description and intended scope of the present invention. When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₅ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

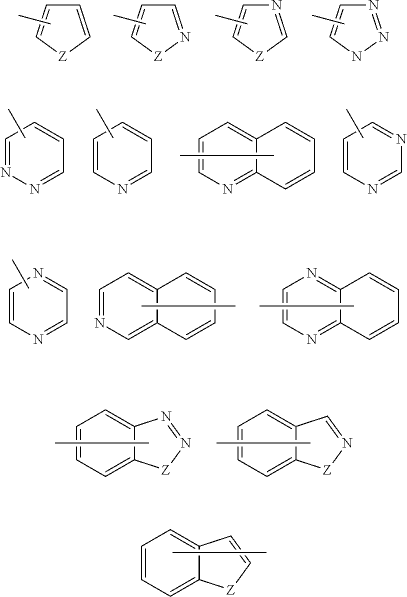

wherein each Z is selected from carbonyl, N, NR⁶⁵, O, and S; and R⁶⁵ is independently hydrogen, C₁-C₈ alkyl, C₃-C₁₀ carbocyclyl, 4-10 membered heterocyclyl, C₆-C₁₀ aryl, and 5-10 membered heteroaryl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₅ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. Cycloalkyl groups can be fused to other cycloalkyl, aryl, or heterocyclyl groups. In certain embodiments, the cycloalkyl group is not substituted, i.e., it is unsubstituted.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

As used herein, “heterocylene” refers to a divalent radical of a heterocycle.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g., heteroaryl; and the like having from 1 to 5, and particularly from 1 to 3 heteroatoms.

As used herein, “cyano” refers to —CN.

As used herein, “halo” or “halogen” refers to fluoro (F), chloro (Cl), bromo (Br) and iodo (I). In certain embodiments, the halo group is either fluoro or chloro.

As used herein, “haloalkyl” refers to an alkyl group substituted with one or more halogen atoms.

As used herein, “nitro” refers to —NO₂.

As used herein, “oxo” refers to —C═O.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NRbb)R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{aa})₂, —P(═O)(NR)₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19, and Gould, Salt selection for basic drugs, International Journal of Pharmaceutics, 33 (1986) 201-217. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, “prophylactic treatment” contemplates an action that occurs before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, a “disease or condition associated with a gain-of-function mutation in KCNT1” refers to a disease or condition that is associated with, is partially or completely caused by, or has one or more symptoms that are partially or completely caused by, a mutation in KCNT1 that results in a gain-of-function phenotype, i.e. an increase in activity of the potassium channel encoded by KCNT1 resulting in an increase in whole cell current. As used herein, a “gain-of-function mutation” is a mutation in KCNT1 that results in an increase in activity of the potassium channel encoded by KCNT1. Activity can be assessed by, for example, ion flux assay or electrophysiology (e.g. using the whole cell patch clamp technique). Typically, a gain-of-function mutation results in an increase of at least or about 20%, 30%, 40%, 50%, 60%, 70%, 80%, 90%, 100%, 125%, 150%, 175%, 200%, 225%, 250%, 275%, 300%, 325%, 350%, 375%, 400% or more compared to the activity of a potassium channel encoded by a wild-type KCNT1.

### Compounds and Compositions

In one aspect, the present disclosure provides a compound of Formula (A):

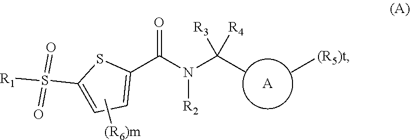

or a pharmaceutically acceptable salt thereof, wherein

A is phenyl or pyridyl;

R₁ is selected from the group consisting of C₁₋₆alkyl, C₃₋₈cycloalkyl, and —NHRₐ, wherein the C₁₋₆alkyl is optionally substituted with C₁₋₆alkoxy;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen or C₁₋₆alkyl;

R₃ and R₄ are each independently selected from the group consisting of hydrogen, C₁₋₆alkyl, C₁₋₆alkylene-O—C₁₋₆alkyl, and C₁₋₆alkoxy;

R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R_{c} and R_{d} are each independently hydrogen or C₁₋₆alkyl;

R₆ is C₁₋₆alkyl or C₁₋₆alkoxy;

t is 0, 1, 2, 3, or 4; and

m is 0, 1, or 2.

In one aspect, the present disclosure provides a compound of Formula (A-1), Formula (A-2), or Formula (A-3):

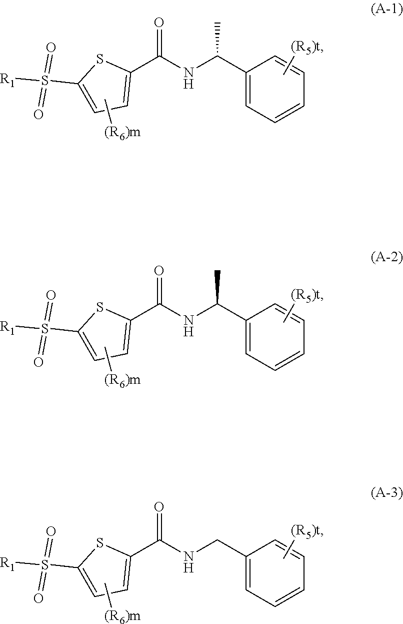

or a pharmaceutically acceptable salt thereof, wherein

R₁ is selected from the group consisting of C₁₋₆alkyl, C₃₋₈cycloalkyl, and —NHRₐ, wherein the C₁₋₆alkyl optionally substituted with C₁₋₆alkoxy;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R_{c} and R_{d} are each independently hydrogen or C₁₋₆alkyl;

R₆ is C₁₋₆alkyl or C₁₋₆alkoxy;

t is 0, 1, 2, 3, or 4; and

m is 0, 1, or 2.

In one aspect, the present disclosure provides a compound of Formula (I):

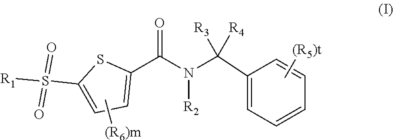

or a pharmaceutically acceptable salt thereof, wherein

R₁ is selected from the group consisting of C₁₋₆alkyl, C₃₋₈cycloalkyl, and —NHRₐ;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen;

R₃ and R₄ are each independently selected from the group consisting of hydrogen, C₁₋₆alkyl, C₁₋₆alkylene-O—C₁₋₆alkyl, and C₁₋₆alkoxy;

R₅ is each independently selected from the group consisting of halogen, —OH, C₁₋₆alkyl, C₁₋₆haloalkyl, C₁₋₆alkoxy, C₁₋₆haloalkoxy, C₁₋₆alkylene-O—C₁₋₆alkyl, and C₃₋₈cycloalkyl;

R₆ is C₁₋₆alkyl or C₁₋₆alkoxy;

t is 0, 1, 2, 3, or 4; and

m is 0, 1, or 2.

In one aspect, provided herein is a a compound of Formula (I-a) or Formula (I-b):

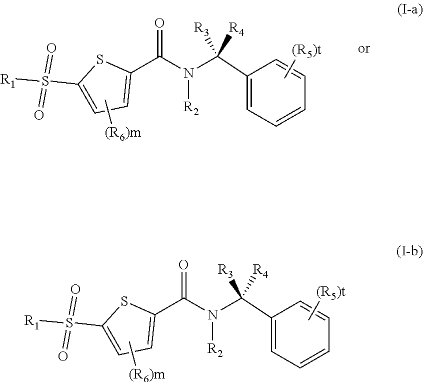

or a pharmaceutically acceptable salt thereof, wherein

R₁ is selected from the group consisting of C₁₋₆alkyl, C₃₋₈cycloalkyl, and —NHRₐ;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen;

R₃ is hydrogen;

R₄ is C₁₋₆alkyl;

R₅ is each independently selected from the group consisting of halogen, —OH, C₁₋₆ alkyl, C₁₋₆haloalkyl, C₁₋₆alkoxy, C₁₋₆haloalkoxy, C₁₋₆alkylene-O—C₁₋₆alkyl, and C₃₋₈cycloalkyl;

R₆ is C₁₋₆alkyl or C₁₋₆alkoxy;

t is 0, 1, 2, 3, or 4; and

m is 0, 1, or 2.

In some embodiments of a compound of Formula (A), (A-1), (A-2), (A-3), (I), (I-a), or (I-b), R₁ is C₁₋₆alkyl or —NHRₐ. In some embodiments of a compound of Formula (A), (A-1), (A-2), (A-3), (I), (I-a), or (I-b), R₁ is C₁₋₆alkyl. In some embodiments of a compound of Formula (A), (A-1), (A-2), (A-3), (I), (I-a), or (I-b), R₁ is methyl, ethyl, or isopropyl. In some embodiments of a compound of Formula (A), (A-1), (A-2), (A-3), (I), (I-a), or (I-b), R₁ is —NHRₐ. In some embodiments of a compound of Formula (A), (A-1), (A-2), (A-3), (I), (I-a), or (I-b), Rₐ is C₁₋₆alkyl. In some embodiments of a compound of Formula (A), (A-1), (A-2), (A-3), (I), (I-a), or (I-b), Rₐ is methyl, ethyl, or isopropyl. In some embodiments of a compound of Formula (A), (A-1), (A-2), (A-3), (I), (I-a), or (I-b), Rₐ is methyl. In some embodiments of a compound of Formula (A), (A-1), (A-2), (A-3), (I), (I-a), or (I-b), R₁ is C₃₋₈cycloalkyl. In some embodiments of a compound of Formula (A), (A-1), (A-2), (A-3), (I), (I-a), or (I-b), R₁ is cyclopropyl, cyclobutyl, cyclopentyl, or cyclohexyl. In some embodiments of a compound of Formula (A), (A-1), (A-2), (A-3), (I), (I-a), or (I-b), R₁ is cyclopropyl. In some embodiments of a compound of Formula (A), (A-1), (A-2), or (A-3), R₁ is C₁₋₆alkyl substituted with C₁₋₆alkoxy. In some embodiments of a compound of Formula (A), (A-1), (A-2), or (A-3), R₁ is C₁₋₆alkyl substituted with —OCH₃.

In some embodiments of Formula (A), (A-1), (A-2), (A-3), (I), (I-a), or (I-b), R₃ is hydrogen. In some embodiments of Formula (A), (A-1), (A-2), (A-3), (I), (I-a), or (I-b), R₄ is hydrogen. In some embodiments of Formula (A), (I), (I-a), or (I-b), R₄ is methyl, ethyl, or isopropyl. In some embodiments of Formula (A), (I), (I-a), or (I-b), R₄ is methyl.

In some embodiments of a compound of Formula (A), (A-1), (A-2), (A-3), (I), (I-a), or (I-b), R₅ is each independently selected from the group consisting of halogen, cyano, —OH, C₁₋₆alkyl, C₁₋₆haloalkyl, C₁₋₆alkoxy, and C₃₋₈cycloalkyl, wherein the C₁₋₆alkyl or C₃₋₈cycloalkyl is optionally substituted with halogen, cyano, or C₁₋₆haloalkyl. In some embodiments of a compound of Formula (A), (A-1), (A-2), (A-3), (I), (I-a), or (I-b), R₅ is each independently selected from the group consisting of chloro, fluoro, bromo, cyano, —OH, methyl, ethyl, isopropyl, tert-butyl, —CHCF₂, —CF₃, —OCH₃, —OCH₂CH₃, —OCH(CH₃)₂, —OCH₂CF₃, and cyclopropyl optionally substituted with —CF₃.

In some embodiments of a compound of Formula (A), (A-1), (A-2), (A-3), (I), (I-a), or (I-b), t is 1 or 2. In some embodiments of a compound of Formula (A), (A-1), (A-2), (A-3), (I), (I-a), or (I-b), t is 1. In some embodiments of a compound of Formula (A), (A-1), (A-2), (A-3), (I), (I-a), or (I-b), t is 2.

In some embodiments of a compound of Formula (I), (I-a), or (I-b), R₁ is C₁₋₆alkyl or —NHRₐ. In some embodiments of a compound of Formula (I), (I-a), or (I-b), Riis C₁₋₆alkyl. In some embodiments of a compound of Formula (I), (I-a), or (I-b), R₁ is methyl. In some embodiments of a compound of Formula (I), (I-a), or (I-b), R₁ is —NHRₐ.

In some embodiments of a compound of Formula (I), (I-a), or (I-b), Rₐ is C₁₋₆alkyl. In some embodiments of a compound of Formula (I), (I-a), or (I-b), Rₐ is methyl.

In some embodiments of a compound of Formula (I), (I-a), or (I-b), R₃ is hydrogen.

In some embodiments of a compound of Formula (I), (I-a), or (I-b), R₄ is hydrogen or methyl.

In some embodiments of a compound of Formula (I), (I-a), or (I-b), R₅ is each independently selected from the group consisting of halogen, —OH, C₁₋₆alkyl, C₁₋₆haloalkyl, C₁₋₆alkoxy, and C₃₋₈cycloalkyl. In some embodiments of a compound of Formula (I), (I-a), or (I-b), R₅ is each independently selected from the group consisting of chloro, fluoro, bromo, —OH, methyl, —CF₃, —OCH₃, and cyclopropyl.

In some embodiments of a compound of Formula (I), (I-a), or (I-b), t is 1 or 2.

In some embodiments of a compound of Formula (I), (I-a), or (I-b), m is 0.

In another aspect, provided herein is a compound of Formula II.

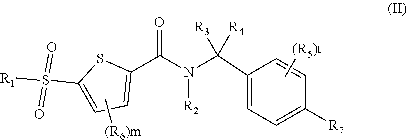

or a pharmaceutically acceptable salt thereof, wherein

R₁ is selected from the group consisting of C₁₋₆alkyl, C₃₋₈cycloalkyl, and —NHRₐ, wherein the C₁₋₆alkyl optionally substituted with C₁₋₆alkoxy;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen;

R₃ and R₄ are each independently selected from the group consisting of hydrogen, C₁₋₆alkyl, C₁₋₆alkylene-O—C₁₋₆alkyl, and C₁₋₆alkoxy;

R₅ is each independently selected from the group consisting of halogen, cyano, —OH, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R₆ is C₁₋₆alkyl or C₁₋₆alkoxy;

R₇ is selected from the group consisting of halogen, cyano, —NR_{c}R_{d}, C₁₋₆alkyl, C₁₋₆haloalkyl, C₁₋₆alkoxy, C₁₋₆haloalkoxy, and C₃₋₈cycloalkyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R_{c} and R_{d} are each independently hydrogen or C₁₋₆alkyl;

t is 0, 1, 2, or 3; and

m is 0, 1, or 2.

In another aspect, provided herein is a compound of Formula (II):

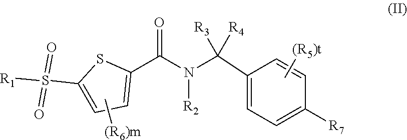

or a pharmaceutically acceptable salt thereof, wherein

R₁ is selected from the group consisting of C₁₋₆alkyl, C₃₋₈cycloalkyl, and —NHRₐ;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen;

R₃ and R₄ are each independently selected from the group consisting of hydrogen, C₁₋₆ alkyl, C₁₋₆alkylene-O—C₁₋₆alkyl, and C₁₋₆alkoxy;

R₅ is each independently selected from the group consisting of halogen, —OH, C₁₋₆alkyl, C₁₋₆haloalkyl, C₁₋₆alkoxy, C₁₋₆haloalkoxy, C₁₋₆alkylene-O—C₁₋₆alkyl, and C₃₋₈-cycloalkyl;

R₆ is C₁₋₆alkyl or C₁₋₆alkoxy;

R₇ is halogen;

t is 0, 1, 2, or 3; and

m is 0, 1, or 2.

In another aspect, provided herein is a compound of Formula (II-a):

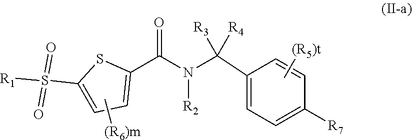

or a pharmaceutically acceptable salt thereof, wherein

R₁ is selected from the group consisting of C₁₋₆alkyl, C₃₋₈cycloalkyl, and —NHRₐ;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen;

R₃ and R₄ are each independently selected from the group consisting of hydrogen, C₁₋₆alkyl, C₁₋₆alkylene-O—C₁₋₆alkyl, and C₁₋₆alkoxy;

R₅ is each independently selected from the group consisting of halogen, —OH, C₁₋₆alkyl, C₁₋₆haloalkyl, C₁₋₆alkoxy, C₁₋₆haloalkoxy, C₁₋₆alkylene-O—C₁₋₆alkyl, and C₃₋₈ cycloalkyl;

R₆ is C₁₋₆alkyl or C₁₋₆alkoxy;

R₇ is halogen;

t is 0, 1, 2, or 3; and

m is 0, 1, or 2.

In another aspect, provided herein is a compound of Formula (II-b):

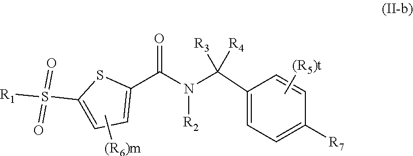

or a pharmaceutically acceptable salt thereof, wherein

R₁ is selected from the group consisting of C₁₋₆alkyl, C₃₋₈cycloalkyl, and —NHRₐ, wherein the C₁₋₆alkyl optionally substituted with C₁₋₆alkoxy;

Rₐ is C₁₋₆alkyl;

R₂ is hydrogen;

R₃ and R₄ are each independently selected from the group consisting of hydrogen, C₁₋₆alkyl, C₁₋₆alkylene-O—C₁₋₆alkyl, and C₁₋₆alkoxy;

R₅ is each independently selected from the group consisting of halogen, cyano, —OH, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R₆ is C₁₋₆alkyl or C₁₋₆alkoxy;

R₇ is selected from the group consisting of halogen, cyano, —NR_{c}R_{d}, C₁₋₆alkyl, C₁₋₆haloalkyl, C₁₋₆alkoxy, C₁₋₆haloalkoxy, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R_{c} and R_{d} are each independently hydrogen or C₁₋₆alkyl;

t is 0, 1, 2, or 3; and

m is 0, 1, or 2.

In some embodiments of a compound of Formula (II), (II-a), or (II-b), R₁ is C₁₋₆alkyl or —NHRₐ. In some embodiments of a compound of Formula (II), (II-a), or (II-b), R₁ is C₁₋₆ alkyl. In some embodiments of a compound of Formula (II), (II-a), or (II-b), R₁ is methyl, ethyl, or isopropyl. In some embodiments of a compound of Formula (II), (II-a), or (II-b), R₁ is methyl. In some embodiments of a compound of Formula (II), (II-a), or (II-b), R₁ is —NHRₐ. In some embodiments of a compound of Formula (II), (II-a), or (II-b), Rₐ is C₁₋₆alkyl. In some embodiments of a compound of Formula (II), (II-a), or (II-b), Rₐ is methyl, ethyl, or isopropyl. In some embodiments of a compound of Formula (II), (II-a), or (II-b), Rₐ is methyl. In some embodiments of a compound of Formula (II), (II-a), or (II-b), R₁ is C₃₋₈cycloalkyl. In some embodiments of a compound of Formula (II), (II-a), or (II-b), R₁ is cyclopropyl, cyclobutyl, cyclopentyl, or cyclohexyl. In some embodiments of a compound of Formula (II), (II-a), or (II-b), R₁ is cyclopropyl. In some embodiments of a compound of Formula (II), (II-a), or (II-b), R₁ is C₁₋₆alkyl substituted with C₁₋₆alkoxy. In some embodiments of a compound of Formula (II), (II-a), or (II-b), R₁ is C₁₋₆alkyl substituted with —OCH₃.

In some embodiments of a compound of Formula (II), (II-a), or (II-b), R₃ is hydrogen.

In some embodiments of a compound of Formula (II), (II-a), or (II-b), R₄ is hydrogen or methyl. In some embodiments of a compound of Formula (II), (II-a), or (II-b), R₄ is hydrogen. In some embodiments of a compound of Formula (II), (II-a), or (II-b), R₄ is methyl. In some embodiments of a compound of Formula (II), (II-a), or (II-b), R₃ and R₄ are hydrogen.

In some embodiments of a compound of Formula (II), (II-a), or (II-b), R₅ is each independently selected from the group consisting of halogen, cyano, —OH, C₁₋₆alkyl, C₁₋₆haloalkyl, C₁₋₆alkoxy, and C₃₋₈cycloalkyl, wherein the C₁₋₆alkyl or C₃₋₈cycloalkyl is optionally substituted with halogen, cyano, or C₁₋₆haloalkyl. In some embodiments of a compound of Formula (II), (II-a), or (II-b), R₅ is each independently selected from the group consisting of chloro, fluoro, bromo, cyano, —OH, methyl, ethyl, isopropyl, tert-butyl, —CHCF₂, —CF₃, —OCH₃, —OCH₂CH₃, —OCH(CH₃)₂, —OCH₂CF₃, and cyclopropyl optionally substituted with —CF₃.

In some embodiments of a compound of Formula (II), (II-a), or (II-b), R₅ is each independently selected from the group consisting of halogen, —OH, C₁₋₆alkyl, C₁₋₆haloalkyl, C₁₋₆alkoxy, and C₃₋₈cycloalkyl. In some embodiments of a compound of Formula (II), (II-a), or (II-b), R₅ is each independently selected from the group consisting of chloro, fluoro, bromo, —OH, methyl, —CF₃, —OCH₃, and cyclopropyl.

In some embodiments of a compound of Formula (II), (II-a), or (II-b), R₇ is selected from the group consisting of halogen, cyano, —NR_{c}R_{d}, C₁₋₆alkyl optionally substituted with cyano, C₁₋₆haloalkyl, C₁₋₆alkoxy, C₁₋₆haloalkoxy, and C₃₋₈cycloalkyl optionally substituted with C₁₋₆haloalkyl. In some embodiments of a compound of Formula (II), (II-a), or (II-b), R₇ is selected from the group consisting of chloro, bromo, cyano, methyl, ethyl, isopropyl, tert-butyl, —CHCF₂, —CF₃, —OCH₂CH₃, —OCH₂CF₃, and cyclopropyl optionally substituted with —CF₃. In some embodiments of a compound of Formula (II), (II-a), or (II-b), R₇ is 4-8 membered heterocyclyl. In some embodiments, the 4-8 membered heterocyclyl comprises one nitrogen.

In some embodiments of a compound of Formula (II), (II-a), or (II-b), t is 1 or 2. In some embodiments of a compound of Formula (II), (II-a), or (II-b), t is 0. In some embodiments of a compound of Formula (II), (II-a), or (II-b), t is 1. In some embodiments of a compound of Formula (II), (II-a), or (II-b), t is 2.

In some embodiments of a compound of Formula (II), (II-a), or (II-b), m is 0.

In some embodiments of a compound of Formula (II) or (II-a), R₁ is C₁₋₆alkyl or —NHRₐ. In some embodiments of a compound of Formula (II) or (II-a), R₁ is C₁₋₆alkyl. In some embodiments of a compound of Formula (II) or (II-a), R₁ is methyl. In some embodiments of a compound of Formula (II) or (II-a), R₁ is —NHRₐ.

In some embodiments of a compound of Formula (II) or (II-a), Rₐ is C₁₋₆alkyl. In some embodiments of a compound of Formula (II) or (II-a), Rₐ is methyl.

In some embodiments of a compound of Formula (II) or (II-a), R₃ is hydrogen.

In some embodiments of a compound of Formula (II) or (II-a), R₄ is hydrogen or methyl.

In some embodiments of a compound of Formula (II) or (II-a), R₅ is each independently selected from the group consisting of halogen, —OH, C₁₋₆alkyl, C₁₋₆haloalkyl, C₁₋₆alkoxy, and C₃₋₈cycloalkyl. In some embodiments of a compound of Formula (II) or (II-a), R₅ is each independently selected from the group consisting of chloro, fluoro, bromo, —OH, methyl, —CF₃, —OCH₃, and cyclopropyl.

In some embodiments of a compound of Formula (II) or (II-a), t is 1 or 2.

In another aspect, provided herein is a compound of Formula (III):

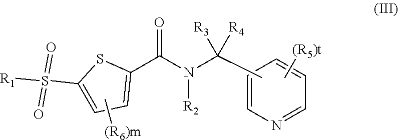

or a pharmaceutically acceptable salt thereof, wherein

R₁ is selected from the group consisting of C₁₋₆alkyl, C₃₋₈cycloalkyl, and —NHRₐ, wherein the C₁₋₆alkyl optionally substituted with C₁₋₆alkoxy;

Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;

R₂ is hydrogen;

R₃ and R₄ are each independently selected from the group consisting of hydrogen, C₁₋₆alkyl, C₁₋₆alkylene-O—C₁₋₆alkyl, and C₁₋₆alkoxy;

R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;

R_{c} and R_{d} are each independently hydrogen or C₁₋₆alkyl;

R₆ is C₁₋₆alkyl or C₁₋₆alkoxy;

t is 0, 1, 2, or 3; and

m is 0, 1, or 2.

In some embodiments of a compound of Formula (III), R₁ is C₁₋₆alkyl. In some embodiments of a compound of Formula (III), R₁ is methyl, ethyl, or isopropyl. In some embodiments of a compound of Formula (III), R₁ is methyl.

In some embodiments of a compound of Formula (III), R₃ is hydrogen.

In some embodiments of a compound of Formula (III), R₄ is hydrogen.

In some embodiments of a compound of Formula (III), R₅ is each independently selected from the group consisting of halogen, cyano, —OH, C₁₋₆alkyl, C₁₋₆haloalkyl, C₁₋₆alkoxy, and C₃₋₈cycloalkyl, wherein the C₁₋₆alkyl or C₃₋₈cycloalkyl is optionally substituted with halogen, cyano, or C₁₋₆haloalkyl. In some embodiments of a compound of Formula (III), R₅ is —CF₃.

In some embodiments of a compound of Formula (III), t is 1. In some embodiments of a compound of Formula (III), t is 2. In some embodiments of a compound of Formula (III), t is 0.

In some embodiments of a compound of Formula (III), m is 0.

In another aspect, provided herein is a pharmaceutical composition comprising a compound of Formula (A), or a pharmaceutically acceptable salt thereof, and a pharmaceutically acceptable excipient.

In another aspect, provided herein is a pharmaceutical composition comprising a compound of Formula (A-1), Formula (A-2), of Formula (A-3), or a pharmaceutically acceptable salt thereof, and a pharmaceutically acceptable excipient.

In another aspect, provided herein is a pharmaceutical composition comprising a compound of Formula (I) or a pharmaceutically acceptable salt thereof and a pharmaceutically acceptable excipient.

In another aspect, provided herein is a pharmaceutical composition comprising a compound of Formula (I-a) or Formula (I-b) or a pharmaceutically acceptable salt thereof and a pharmaceutically acceptable excipient.

In another aspect, provided herein is a pharmaceutical composition comprising a compound of Formula (II) or a pharmaceutically acceptable salt thereof and a pharmaceutically acceptable excipient.

In another aspect, provided herein is a pharmaceutical composition comprising a compound of Formula (II-a) or a pharmaceutically acceptable salt thereof and a pharmaceutically acceptable excipient.

In another aspect, provided herein is a pharmaceutical composition comprising a compound of Formula (II-b) or a pharmaceutically acceptable salt thereof and a pharmaceutically acceptable excipient.

In another aspect, provided herein is a pharmaceutical composition comprising a compound of Formula (III) or a pharmaceutically acceptable salt thereof and a pharmaceutically acceptable excipient.

In typical embodiments, the present invention is intended to encompass the compounds disclosed herein, and the pharmaceutically acceptable salts, tautomeric forms, polymorphs, and prodrugs of such compounds. In some embodiments, the present invention includes a pharmaceutically acceptable addition salt, a pharmaceutically acceptable ester, a solvate (e.g., hydrate) of an addition salt, a tautomeric form, all polymorphs including polymorphs of hydrates and solvates, an enantiomer, a mixture of enantiomers, a diastereomer, a mixture of diastereomers, a stereoisomer or mixture of stereoisomers (pure or as a racemic or non-racemic mixture) of a compound described herein, e.g., a compound of Formula (A), (A-1), (A-2), (A-3), (I), (I-a), (I-b), (II), (II-a), (II-b), or (III).

Provided herein is a compound is selected from the group consisting of:

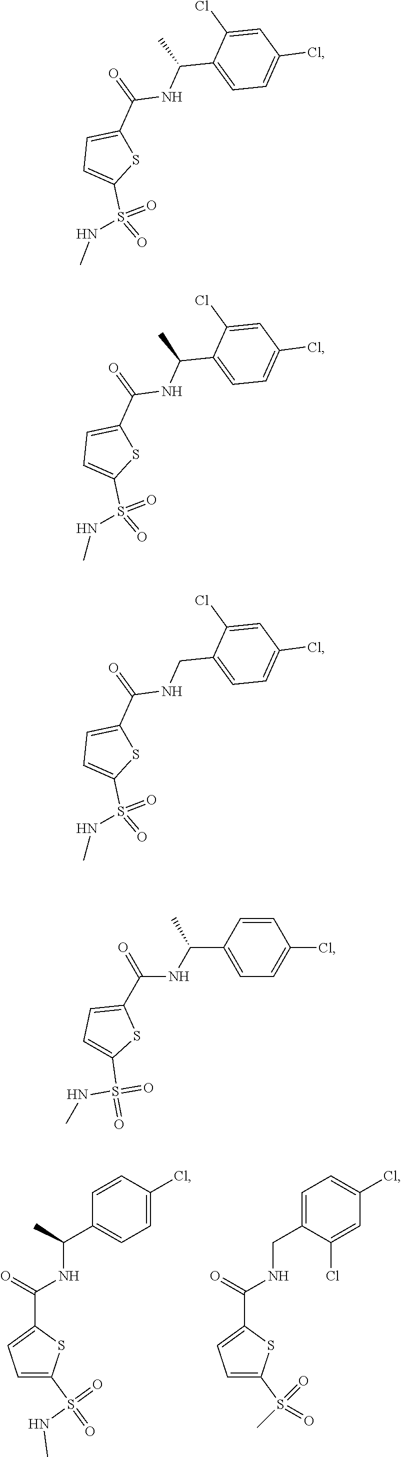 [SMILES: CNS(=O)(=O)c1ccc(C(=O)N[C@H](C)c2ccc(Cl)cc2Cl)s1 ; CNS(=O)(=O)c1ccc(C(=O)N[C@@H](C)c2ccc(Cl)cc2Cl)s1 ; CNS(=O)(=O)c1ccc(C(=O)NCc2ccc(Cl)cc2Cl)s1 ; CNS(=O)(=O)c1ccc(C(=O)N[C@H](C)c2ccc(Cl)cc2)s1 ; CNS(=O)(=O)c1ccc(C(=O)N[C@@H](C)c2ccc(Cl)cc2)s1 ; CS(=O)(=O)c1ccc(C(=O)NCc2ccc(Cl)cc2Cl)s1]

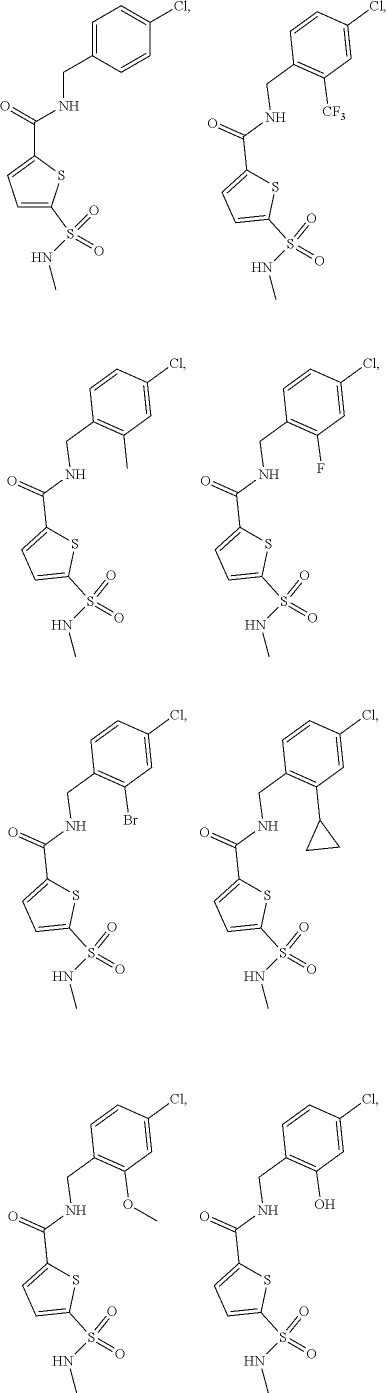

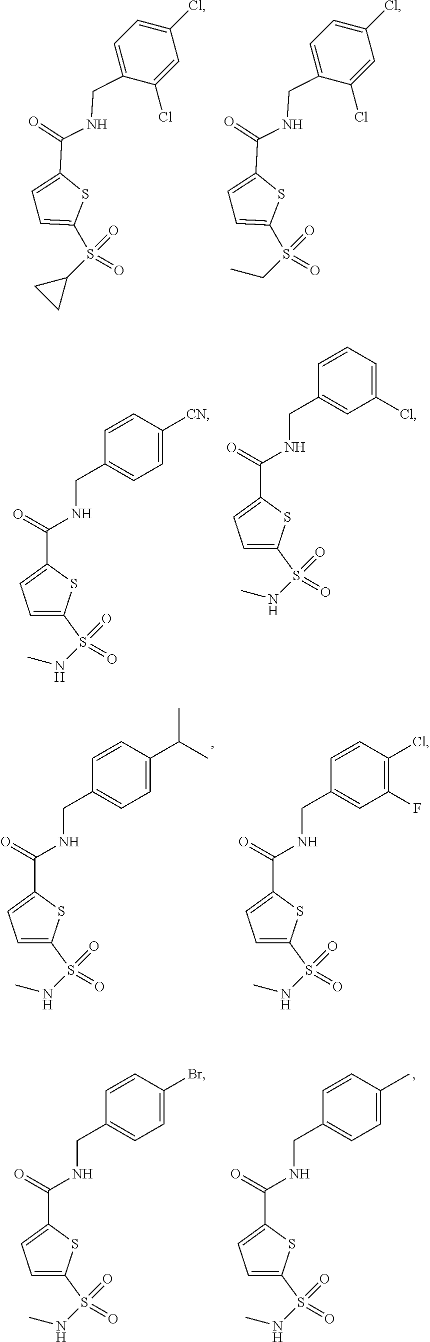

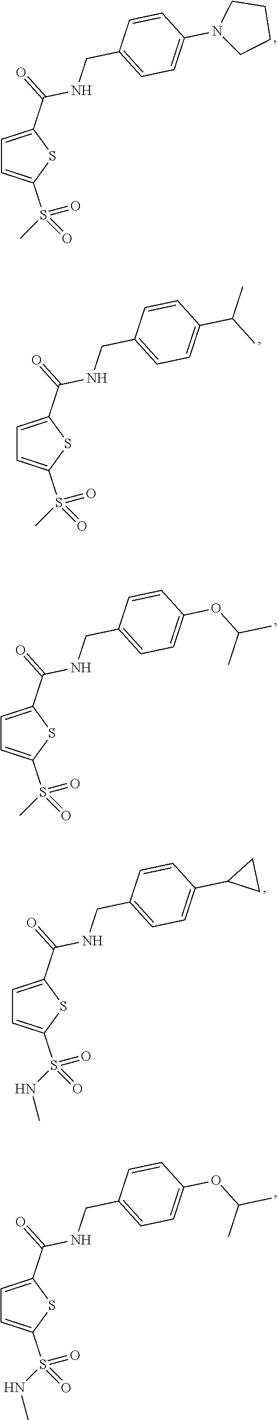 [SMILES: CS(=O)(=O)c1ccc(C(=O)NCc2ccc(N3CCCC3)cc2)s1 ; CC(C)c1ccc(CNC(=O)c2ccc(S(C)(=O)=O)s2)cc1 ; CC(C)Oc1ccc(CNC(=O)c2ccc(S(C)(=O)=O)s2)cc1 ; CNS(=O)(=O)c1ccc(C(=O)NCc2ccc(C3CC3)cc2)s1 ; CNS(=O)(=O)c1ccc(C(=O)NCc2ccc(OC(C)C)cc2)s1]

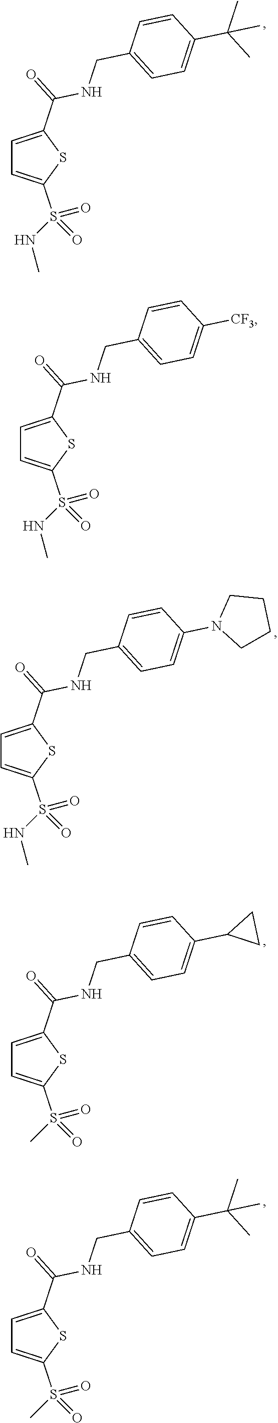 [SMILES: CNS(=O)(=O)c1ccc(C(=O)NCc2ccc(C(C)(C)C)cc2)s1 ; CNS(=O)(=O)c1ccc(C(=O)NCc2ccc(C(F)(F)F)cc2)s1 ; CNS(=O)(=O)c1ccc(C(=O)NCc2ccc(N3CCCC3)cc2)s1 ; CS(=O)(=O)c1ccc(C(=O)NCc2ccc(C3CC3)cc2)s1 ; CC(C)(C)c1ccc(CNC(=O)c2ccc(S(C)(=O)=O)s2)cc1]

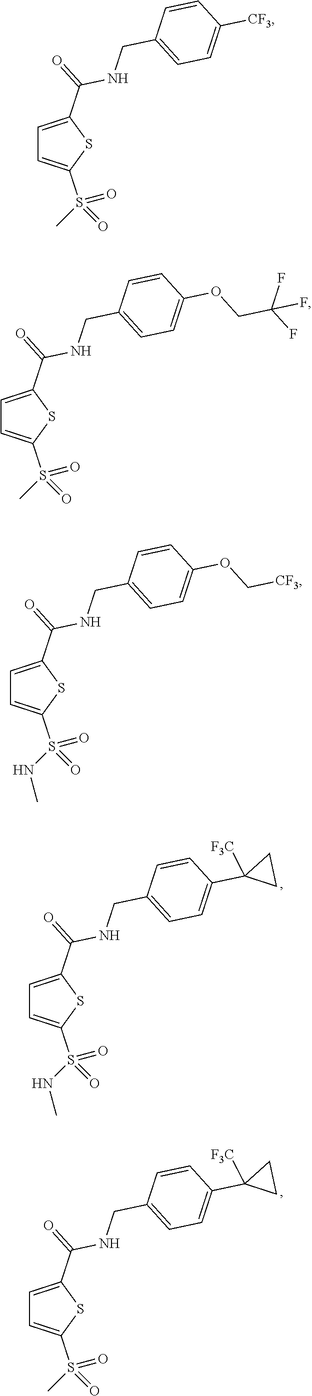 [SMILES: CS(=O)(=O)c1ccc(C(=O)NCc2ccc(C(F)(F)F)cc2)s1 ; CS(=O)(=O)c1ccc(C(=O)NCc2ccc(OCC(F)(F)F)cc2)s1 ; CNS(=O)(=O)c1ccc(C(=O)NCc2ccc(OCC(F)(F)F)cc2)s1 ; CNS(=O)(=O)c1ccc(C(=O)NCc2ccc(C3(C(F)(F)F)CC3)cc2)s1 ; CS(=O)(=O)c1ccc(C(=O)NCc2ccc(C3(C(F)(F)F)CC3)cc2)s1]

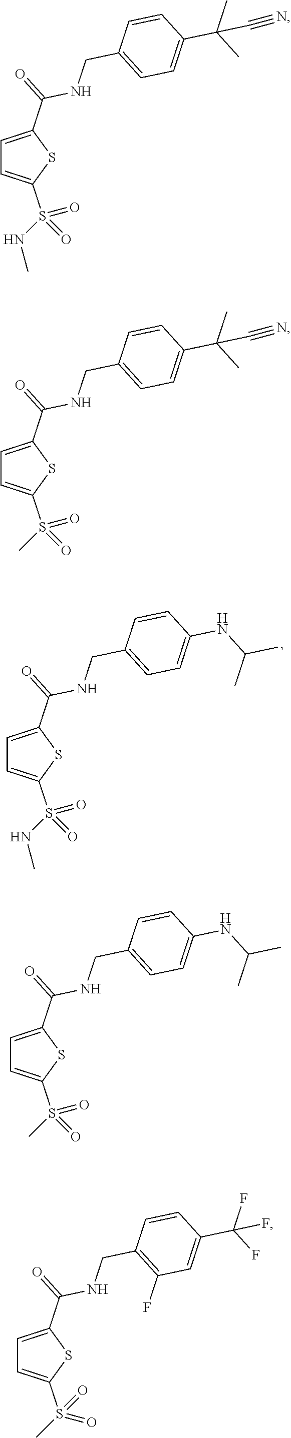 [SMILES: CNS(=O)(=O)c1ccc(C(=O)NCc2ccc(C(C)(C)C#N)cc2)s1 ; CC(C)(C#N)c1ccc(CNC(=O)c2ccc(S(C)(=O)=O)s2)cc1 ; CNS(=O)(=O)c1ccc(C(=O)NCc2ccc(NC(C)C)cc2)s1 ; CC(C)Nc1ccc(CNC(=O)c2ccc(S(C)(=O)=O)s2)cc1 ; CS(=O)(=O)c1ccc(C(=O)NCc2ccc(C(F)(F)F)cc2F)s1]

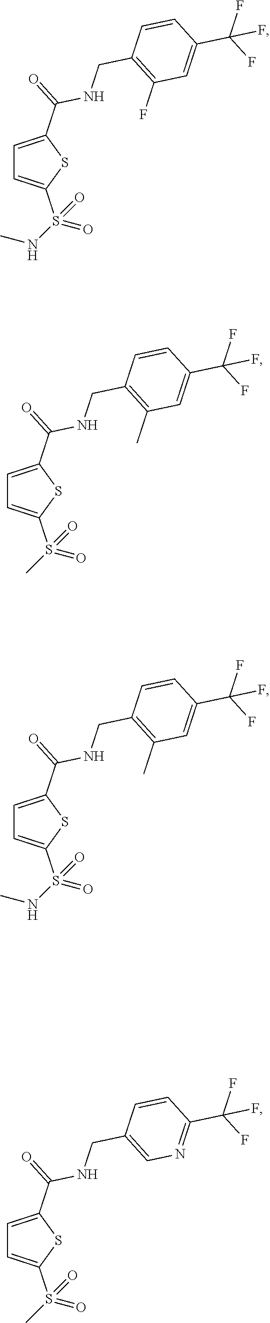 [SMILES: CNS(=O)(=O)c1ccc(C(=O)NCc2ccc(C(F)(F)F)cc2F)s1 ; Cc1cc(C(F)(F)F)ccc1CNC(=O)c1ccc(S(C)(=O)=O)s1 ; CNS(=O)(=O)c1ccc(C(=O)NCc2ccc(C(F)(F)F)cc2C)s1 ; CS(=O)(=O)c1ccc(C(=O)NCc2ccc(C(F)(F)F)nc2)s1]

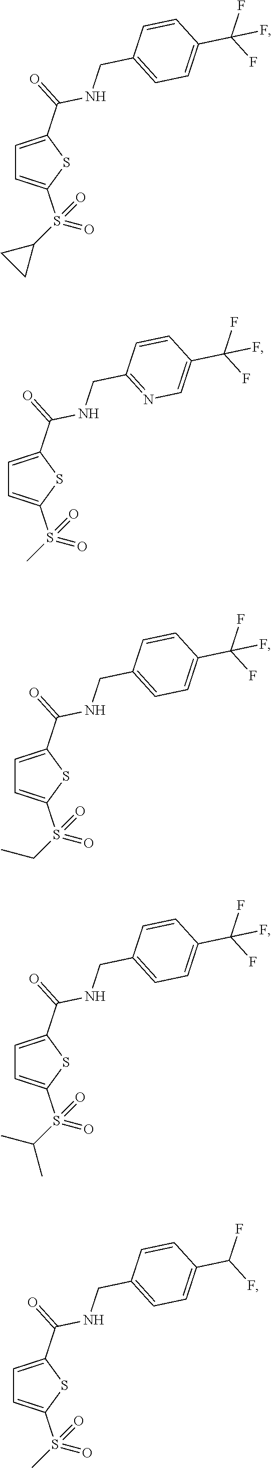 [SMILES: O=C(NCc1ccc(C(F)(F)F)cc1)c1ccc(S(=O)(=O)C2CC2)s1 ; CS(=O)(=O)c1ccc(C(=O)NCc2ccc(C(F)(F)F)cn2)s1 ; CCS(=O)(=O)c1ccc(C(=O)NCc2ccc(C(F)(F)F)cc2)s1 ; CC(C)S(=O)(=O)c1ccc(C(=O)NCc2ccc(C(F)(F)F)cc2)s1 ; CS(=O)(=O)c1ccc(C(=O)NCc2ccc(C(F)F)cc2)s1]

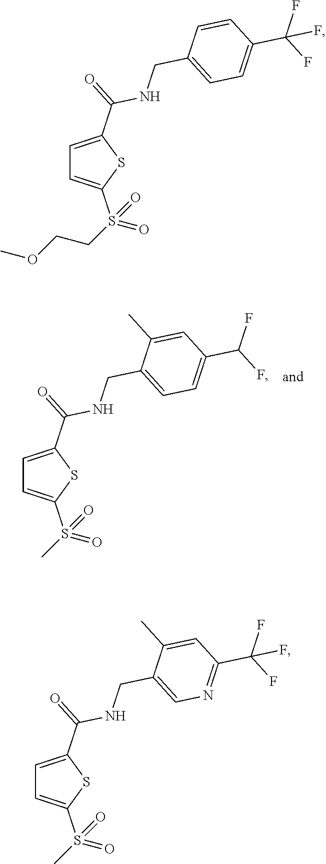 [SMILES: COCCS(=O)(=O)c1ccc(C(=O)NCc2ccc(C(F)(F)F)cc2)s1 ; Cc1cc(C(F)F)ccc1CNC(=O)c1ccc(S(C)(=O)=O)s1 ; Cc1cc(C(F)(F)F)ncc1CNC(=O)c1ccc(S(C)(=O)=O)s1]

or a pharmaceutically acceptable salt thereof.

### General Synthetic Scheme

Exemplary methods for preparing compounds described herein are illustrated in the following synthetic schemes. These schemes are given for the purpose of illustrating the invention, and should not be regarded in any manner as limiting the scope or the spirit of the invention.

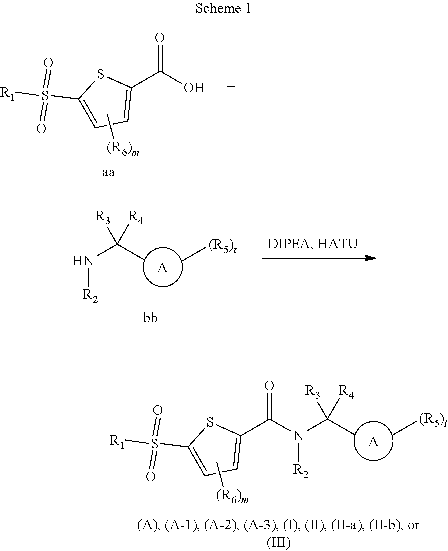

The synthetic route illustrated in Scheme 1 depicts an exemplary procedure for preparing a compound of Formula (A), (A-1), (A-2), (A-3), (I), (I-a), (I-b), (II), (II-a), (II-b), or (III). Coupling of carboxylic acid aa and amine bb using standard peptide coupling procedures (e.g., DIPEA followed by HATU in DCM or DMF) yields a compound of Formula (A), (A-1), (A-2), (A-3), (I), (I-a), (I-b), (II), (II-a), (II-b), or (III).

### Methods of Treatment

The compounds and compositions described above and herein can be used to treat a neurological disease or disorder or a disease or condition associated with excessive neuronal excitability and/or a gain-of-function mutation in a gene (e.g., KCNT1). Exemplary diseases, disorders, or conditions include epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, developmental and epileptic encephalopathy (DEE), early infantile epileptic encephalopathy (EIEE), generalized epilepsy, focal epilepsy, multifocal epilepsy, temporal lobe epilepsy, Ohtahara syndrome, early myoclonic encephalopathy and Lennox Gastaut syndrome, drug resistant epilepsy, seizures (e.g., frontal lobe seizures, generalized tonic clonic seizures, asymmetric tonic seizures, focal seizures), leukodystrophy, hypomyelinating leukodystrophy, leukoencephalopathy, and sudden unexpected death in epilepsy, cardiac dysfunctions (e.g., cardiac arrhythmia, Brugada syndrome, myocardial infarction), pulmonary vasculopathy/hemorrhage, pain and related conditions (e.g. neuropathic pain, acute/chronic pain, migraine, etc), muscle disorders (e.g. myotonia, neuromyotonia, cramp muscle spasms, spasticity), itch and pruritis, movement disorders (e.g., ataxia and cerebellar ataxias), psychiatric disorders (e.g. major depression, anxiety, bipolar disorder, schizophrenia, attention-deficit hyperactivity disorder), neurodevelopmental disorder, learning disorders, intellectual disability, Fragile X, neuronal plasticity, and autism spectrum disorders.

In some embodiments, the neurological disease or disorder or the disease or condition associated with excessive neuronal excitability and/or a gain-of-function mutation in a gene (e.g., KCNT1) is selected from EIMIFS, ADNFLE and West syndrome. In some embodiments, the neurological disease or disorder or the disease or condition associated with excessive neuronal excitability and/or a gain-of-function mutation in a gene (e.g., KCNT1) is selected from infantile spasms, epileptic encephalopathy, focal epilepsy, Ohtahara syndrome, developmental and epileptic encephalopathy and Lennox Gastaut syndrome. In some embodiments, the neurological disease or disorder or the disease or condition associated with excessive neuronal excitability and/or a gain-of-function mutation in a gene (e.g., KCNT1) is seizure. In some embodiments, the neurological disease or disorder or the disease or condition associated with excessive neuronal excitability and/or a gain-of-function mutation in a gene (e.g., KCNT1) is selected from cardiac arrhythmia, Brugada syndrome, and myocardial infarction.

In some embodiments, the neurological disease or disorder or the disease or condition associated with excessive neuronal excitability and/or a gain-of-function mutation in a gene (e.g., KCNT1) is selected from the group consisting of the learning disorders, Fragile X, intellectual function, neuronal plasticity, psychiatric disorders, and autism spectrum disorders.

Accordingly, the compounds and compositions thereof can be administered to a subject with a neurological disease or disorder or a disease or condition associated with excessive neuronal excitability and/or a gain-of-function mutation in a gene such as KCNT1 (e.g., EIMFS, ADNFLE, West syndrome, infantile spasms, epileptic encephalopathy, focal epilepsy, Ohtahara syndrome, developmental and epileptic encephalopathy, and Lennox Gastaut syndrome, seizures, cardiac arrhythmia, Brugada syndrome, and myocardial infarction).

EIMFS is a rare and debilitating genetic condition characterized by an early onset (before 6 months of age) of almost continuous heterogeneous focal seizures, where seizures appear to migrate from one brain region and hemisphere to another. Patients with EIMFS are generally intellectually impaired, non-verbal and non-ambulatory. While several genes have been implicated to date, the gene that is most commonly associated with EIMFS is KCNT1. Several de novo mutations in KCNT1 have been identified in patients with EIMFS, including V271F, G288S, R428Q, R474Q, R474H, R474C, 1760M, A934T, P924L, G243S, H257D, A259D, R262Q, Q270E, L2741, F346L, C377S, R398Q, P409S, A477T, F502V, M516V, Q550del, K629E, K629N, I760F, E893K, M896K, R933G, R950Q, K1154Q (Barcia et al. (2012) Nat Genet. 44: 1255-1260; Ishii et al. (2013) Gene 531:467-471; McTague et al. (2013) Brain. 136: 1578-1591; Epi4K Consortium & Epilepsy Phenome/Genome Project. (2013) Nature 501:217-221; Lim et al. (2016) Neurogenetics; Ohba et al. (2015) Epilepsia 56:el21-el28; Zhou et al. (2018) Genes Brain Behav. e12456; Moller et al. (2015) Epilepsia. el 14-20; Numis et al. (2018) Epilepsia. 1889-1898; Madaan et al. Brain Dev. 40(3):229-232; McTague et al. (2018) Neurology. 90(1):e55-e66; Kawasaki et al. (2017) J Pediatr. 191:270-274; Kim et al. (2014) Cell Rep. 9(5):1661-1672; Ohba et al. (2015) Epilepsia. 56(9):e121-8; Rizzo et al. (2016) Mol Cell Neurosci. 72:54-63; Zhang et al. (2017) Clin Genet. 91(5):717-724; Mikati et al. (2015) Ann Neurol. 78(6):995-9; Baumer et al. (2017) Neurology. 89(21):2212; Dilena et al. (2018) Neurotherapeutics. 15(4):1112-1126). These mutations are gain-of-function, missense mutations that are dominant (i.e. present on only one allele) and result in change in function of the encoded potassium channel that causes a marked increase in whole cell current when tested in Xenopus oocyte or mammalian expression systems (see e.g. Milligan et al. (2015) Ann Neurol. 75(4): 581-590; Barcia et al. (2012) Nat Genet. 44(11): 1255-1259; and Mikati et al. (2015) Ann Neurol. 78(6): 995-999).

ADNFLE has a later onset than EIMFS, generally in mid-childhood, and is generally a less severe condition. It is characterized by nocturnal frontal lobe seizures and can result in psychiatric, behavioural and cognitive disabilities in patients with the condition. While ADNFLE is associated with genes encoding several neuronal nicotinic acetylcholine receptor subunits, mutations in the KCNT1 gene have been implicated in more severe cases of the disease (Heron et al. (2012) Nat Genet. 44: 1188-1190). Functional studies of the mutated KCNT1 genes associated with ADNFLE indicated that the underlying mutations (M896I, R398Q, Y796H and R928C) were dominant, gain-of-function mutations (Milligan et al. (2015) Ann Neurol. 75(4): 581-590; Mikati et al. (2015) Ann Neurol. 78(6): 995-999).

West syndrome is a severe form of epilepsy composed of a triad of infantile spasms, an interictal electroencephalogram (EEG) pattern termed hypsarrhythmia, and mental retardation, although a diagnosis can be made one of these elements is missing. Mutations in KCNT1, including G652V and R474H, have been associated with West syndrome (Fukuoka et al. (2017) Brain Dev 39:80-83 and Ohba et al. (2015) Epilepsia 56:el21-el28). Treatment targeting the KCNT1 channel suggests that these mutations are gain-of-function mutations (Fukuoka et al. (2017) Brain Dev 39:80-83).

In one aspect, the present invention features a method of treating treat a disease or condition associated with excessive neuronal excitability and/or a gain-of-function mutation in a gene such as KCNT1 (for example, epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy (DEE), and Lennox Gastaut syndrome, seizures, leukodystrophy, leukoencephalopathy, intellectual disability, Multifocal Epilepsy, Generalized tonic clonic seizures, Drug resistant epilepsy, Temporal lobe epilepsy, cerebellar ataxia, Asymmetric Tonic Seizures) and cardiac dysfunctions (e.g., cardiac arrhythmia, Brugada syndrome, sudden unexpected death in epilepsy, myocardial infarction), pain and related conditions (e.g. neuropathic pain, acute/chronic pain, migraine, etc), muscle disorders (e.g. myotonia, neuromyotonia, cramp muscle spasms, spasticity), itch and pruritis, ataxia and cerebellar ataxias, psychiatric disorders (e.g. major depression, anxiety, bipolar disorder, schizophrenia), learning disorders, Fragile X, neuronal plasticity, and autism spectrum disorders) comprising administering to a subject in need thereof a compound disclosed herein (e.g., a compound of Formula (A), (A-1), (A-2), (A-3), (I), (I-a), (I-b), (II), (II-a), (II-b), or (III), or a pharmaceutically acceptable salt thereof) or a pharmaceutical composition disclosed herein (e.g., a pharmaceutical composition comprising a compound disclosed herein (e.g., a compound of Formula (A), (A-1), (A-2), (A-3), (I), (I-a), (I-b), (II), (II-a), (II-b), or (III), or a pharmaceutically acceptable salt thereof), and a pharmaceutically acceptable excipient).

In some examples, the subject presenting with a disease or condition that may be associated with a gain-of-function mutation in KCNT1 is genotyped to confirm the presence of a known gain-of-function mutation in KCNT1 prior to administration of the compounds and compositions thereof. For example, whole exome sequencing can be performed on the subject. Gain-of-function mutations associated with EIMFS may include, but are not limited to, V271F, G288S, R428Q, R474Q, R474H, R474C, 1760M, A934T, P924L, G243S, H257D, A259D, R262Q, Q270E, L2741, F346L, C377S, R398Q, P409S, A477T, F502V, M516V, Q550del, K629E, K629N, I760F, E893K, M896K, R933G, R950Q, and K1154Q. Gain-of-function mutations associated with ADNFLE may include, but are not limited to, M896I, R398Q, Y796H, R928C, and G288S. Gain-of-function mutations associated with West syndrome may include, but are not limited to, G652V and R474H. Gain-of-function mutations associated with temporal lobe epilepsy may include, but are not limited to, R133H and R565H. Gain-of-function mutations associated with Lennox-Gastaut may include, but are not limited to, R209C. Gain-of-function mutations associated with seizures may include, but are not limited to, A259D, G288S, R474C, R474H. Gain-of-function mutations associated with leukodystrophy may include, but are not limited to, G288S and Q906H. Gain-of-function mutations associated with Multifocal Epilepsy may include, but are not limited to, V340M. Gain-of-function mutations associated with EOE may include, but are not limited to, F346L and A934T. Gain-of-function mutations associated with Early-onset epileptic encephalopathies (EOEE) may include, but are not limited to, R428Q. Gain-of-function mutations associated with developmental and epileptic encephalopathies may include, but are not limited to, F346L, R474H, and A934T. Gain-of-function mutations associated with epileptic encephalopathies may include, but are not limited to, L437F, Y796H, P924L, R961H. Gain-of-function mutations associated with Early Infantile Epileptic Encephalopathy (EIEE) may include, but are not limited to, M896K. Gain-of-function mutations associated with drug resistant epilepsy and generalized tonic-clonic seizure may include, but are not limited to, F346L. Gain-of-function mutations associated with migrating partial seizures of infancy may include, but are not limited to, R428Q. Gain-of-function mutations associated with Leukoencephalopathy may include, but are not limited to, F932I. Gain-of-function mutations associated with NFLE may include, but are not limited to, A934T and R950Q. Gain-of-function mutations associated with Ohtahara syndrome may include, but are not limited to, A966T. Gain-of-function mutations associated with infantile spasms may include, but are not limited to, P924L. Gain-of-function mutations associated with Brugada Syndrome may include, but are not limited to, R106Q. Gain-of-function mutations associated with Brugada Syndrome may include, but are not limited to, R474H.

In other examples, the subject is first genotyped to identify the presence of a mutation in KCNT1 and this mutation is then confirmed to be a gain-of-function mutation using standard in vitro assays, such as those described in Milligan et al. (2015) Ann Neurol. 75(4): 581-590. Typically, the presence of a gain-of-function mutation is confirmed when the expression of the mutated KCNT1 allele results an increase in whole cell current compared to the whole cell current resulting from expression of wild-type KCNT1 as assessed using whole-cell electrophysiology (such as described in Milligan et al. (2015) Ann Neurol. 75(4): 581-590; Barcia et al. (2012) Nat Genet. 44(11): 1255-1259; Mikati et al. (2015) Ann Neurol. 78(6): 995-999; or Rizzo et al. Mol Cell Neurosci. (2016) 72:54-63). This increase of whole cell current can be, for example, an increase of at least or about 50%, 100%, 150%, 200%, 250%, 300%, 350%, 400% or more. The subject can then be confirmed to have a disease or condition associated with a gain-of-function mutation in KCNT1.

In particular examples, the subject is confirmed as having a KCNT1 allele containing a gain-of-function mutation (e.g. V271F, G288S, R398Q, R428Q, R474Q, R474H, R474C, G652V, 1760M, Y796H, M896I, P924L, R928C or A934T).

The compounds disclosed herein (e.g., a compound of Formula (A), (A-1), (A-2), (A-3), (I), (I-a), (I-b), (II), (II-a), (II-b), or (III), (II-j), (II-k) or a pharmaceutically acceptable salt thereof) or the pharmaceutical composition disclosed herein (e.g., a pharmaceutical composition comprising a compound disclosed herein (e.g., a compound of Formula (A), (A-1), (A-2), (A-3), (I), (I-a), (I-b), (II), (II-a), (II-b), or (III), or a pharmaceutically acceptable salt thereof), and a pharmaceutically acceptable excipient) can also be used therapeutically for conditions associated with excessive neuronal excitability where the excessive neuronal excitability is not necessarily the result of a gain-of-function mutation in KCNT1. Even in instances where the disease is not the result of increased KCNT1 expression and/or activity, inhibition of KCNT1 expression and/or activity can nonetheless result in a reduction in neuronal excitability, thereby providing a therapeutic effect. Thus, the compounds disclosed herein (e.g., a compound of Formula (A), (A-1), (A-2), (A-3), (I), (I-a), (I-b), (II), (II-a), (II-b), or (III), or a pharmaceutically acceptable salt thereof) or the pharmaceutical composition disclosed herein (e.g., a pharmaceutical composition comprising a compound disclosed herein (e.g., a compound of Formula (A), (A-1), (A-2), (A-3), (I), (I-a), (I-b), (II), (II-a), (II-b), or (III), or a pharmaceutically acceptable salt thereof), and a pharmaceutically acceptable excipient) can be used to treat a subject with conditions associated with excessive neuronal excitability, for example, epilepsy and other encephalopathies (e.g., epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, and Lennox Gastaut syndrome, seizures) or cardiac dysfunctions (e.g., cardiac arrhythmia, Brugada syndrome, myocardial infarction), regardless of whether or not the disease or disorder is associated with a gain-of-function mutation in KCNT1.

### Pharmaceutical Compositions and Routes of Administration

Compounds provided in accordance with the present invention, e.g., a compound of Formula (A), (A-1), (A-2), (A-3), (I), (I-a), (I-b), (II), (II-a), (II-b), or (III), or a pharmaceutically acceptable salt thereof, are usually administered in the form of pharmaceutical compositions. This invention therefore provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprising a disclosed compound, or pharmaceutically acceptable salt thereof, and a pharmaceutically acceptable carrier.

### EXAMPLES

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high performance liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

### List of Abbreviations

TEA triethylamine
THE tetrahydrofuran
ACN acetonitrile
DMF N,N-dimethylformamide
DCM dichloromethane
TFA trifluoroacetic acid
HATU hexafluorophosphate azabenzotriazole tetramethyl uranium
DIPEA N,N-diisopropylethylamine
DMSO dimethylsulfoxide
RT room temperature
EtOAc ethyl acetate
m-CPBA meta-Chloroperoxybenzoic acid
DAST diethylaminosulfur trifluoride
LAH lithium aluminum hydride
Pd(PPh₃)₄ tetrakis(triphenylphosphine)palladium(0)

### Example 1. Synthesis of 5-(N-methylsulfamoyl)thiophene-2-carboxylic acid (a3)

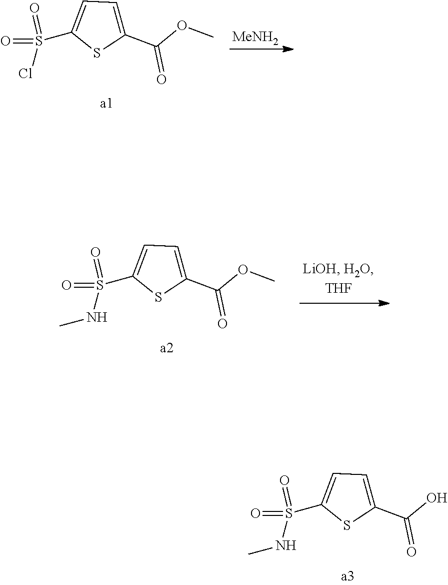

### Synthesis of methyl 5-(chlorosulfonyl)thiophene-2-carboxylate (a1)

Compound a1 was synthesized according to the procedure disclosed in U.S. Patent Application Publication No. 20160200719.

### Synthesis of methyl 5-(N-methylsulfamoyl)thiophene-2-carboxylate (a2)

To a stirred solution of a1 (15 g, 62.32 mmol) in THF (150 mL) were added TEA (26.1 mL, 186.97 mmol) and methanamine (1M in THF, 5.81 g, 186.97 mmol) at 0° C. The reaction mixture was stirred at RT for 16 h. The reaction mixture was quenched with water and extracted with ethyl acetate. The combined organic layer was separated, dried over sodium sulphate and concentration under reduced pressure. The crude compound was purified by column chromatography using 100-200 silica and DCM as an eluent to afford a2 (10 g, 39.3 mmol, 63% yield) as a liquid.

### Synthesis of 5-(N-methylsulfamoyl)thiophene-2-carboxylic acid (a3)

To a stirred solution of a2 (1 g, 4.25 mmol) in THE was added aqueous solution of LiOH (267.51 mg, 6.38 mmol) at 0° C. and the reaction mixture was stirred at RT for 4 h. The volatile solvent was removed under reduced pressure. The residue was diluted with water and extracted with diethyl ether (3×5 mL). The aqueous layer was separated; cooled to 0° C. and acidified with 2N HCl. The precipitated solid was collected by filtration and dried under reduced pressure to afford a3 (700 mg, 3.12 mmol, 74% yield).

### Example 2. General Procedure for Amidation

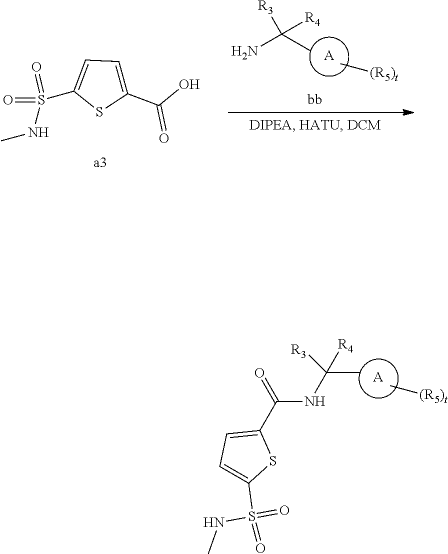

To a stirred solution of acid a3 (1 eq.) and corresponding amine bb (1.1 eq.) in DMF/DCM was added DIPEA (2 eq.) followed by HATU (1.5 eq.) at 0° C. and the resulting reaction mixture was stirred at RT for 16 h. The reaction mixture was diluted with water and extracted with ethyl acetate. The combined organic layer was separated, dried over anhydrous sodium sulphate, filtered and concentrated under reduced pressure to afford the crude compound. The crude compound was purified by silica gel column chromatography/prep. HPLC to afford the desired compound (a compound of Formula (A), (A-1), (A-2), (A-3), (I), (I-a), (I-b), (II), (II-a), (II-b), or (III)).

### Example 3. Synthesis of (R)—N-(1-(2,4-dichlorophenyl)ethyl)-5-(N-methylsulfamoyl)thiophene-2-carboxamide (Compound 1)

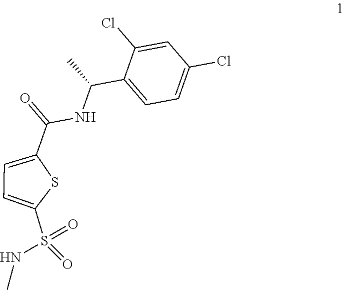 [SMILES: CNS(=O)(=O)c1ccc(C(=O)N[C@H](C)c2ccc(Cl)cc2Cl)s1]

Compound 1 was synthesized according to the procedure described in Example 2. Yield: 58 mg, 0.145 mmol (from 200 mg of a3). HPLC: Rt 8.52 min, 98.1%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 393.10 (M+H), Rt 1.93 min; Column: X-select CSH C18 (3*50) mm, 2.5 μm. ¹H NMR (400 MHz, DMSO-d6) δ_{H} 9.24 (d, 1H), 7.94 (d, 1H), 7.86-7.80 (m, 1H), 7.64-7.58 (m, 2H), 7.54-7.48 (m, 1H), 7.48-7.42 (m, 1H), 5.36-5.30 (m, 1H), 1.44 (d, 3H), 3H merged in solvent peak. Chiral HPLC: Rt 5.36 min, 100%; Method 84076, SFC column: DIACEL CHIRALPAK-IG (150×4.6 mm, 5 um), —Mobile Phase: A) CO₂ B) MeOH+0.1% NH₃, Gradient: 20-40% B in 5 min, hold 40% B till 9 min, 40-20% B in 10 min, hold 20% B till 12 min. Wavelength: 271 nm, Flow: 3 mL/min.

### Example 4. Synthesis of (S)—N-(1-(2,4-dichlorophenyl)ethyl)-5-(N-methylsulfamoyl)thiophene-2-carboxamide (Compound 2)

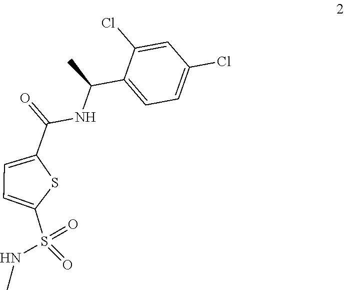 [SMILES: CNS(=O)(=O)c1ccc(C(=O)N[C@@H](C)c2ccc(Cl)cc2Cl)s1]

Compound 2 was synthesized according to the procedure described in Example 2. Yield: 53 mg, 0.135 mmol (from 200 mg of a3). HPLC: Rt 8.52 min, 99.9%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 393.10 (M+H), Rt 1.95 min; Column: X-select CSH C18 (3*50) mm, 2.5 μm. ¹H NMR (400 MHz, DMSO-d6) δ_{H} 9.25 (d, 1H), 7.94 (d, 1H), 7.84-7.80 (m, 1H), 7.64-7.58 (m, 2H), 7.54-7.48 (m, 1H), 7.48-7.42 (m, 1H), 5.36-5.30 (m, 1H), 1.45 (d, 3H), 3H merged in solvent peak. Chiral HPLC: Rt 6.62 min, 99.67%; Method: 84076, SFC column: DIACEL CHIRALPAK-IG (150×4.6 mm, 5 um), —Mobile Phase: A) CO₂ B) MeOH+0.1% NH₃, Gradient: 20-40% B in 5 min, hold 40% B till 9 min, 40-20% B in 10 min, hold 20% B till 12 min. Wavelength: 271 nm, Flow: 3 mL/min.

### Example 5. Synthesis of N-(2,4-dichlorobenzyl)-5-(N-methylsulfamoyl)thiophene-2-carboxamide (Compound 3)

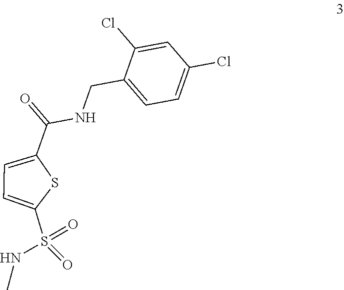 [SMILES: CNS(=O)(=O)c1ccc(C(=O)NCc2ccc(Cl)cc2Cl)s1]

Compound 3 was synthesized according to the procedure described in Example 2. Yield: 25 mg, 0.065 mmol (from 200 mg of a3). HPLC: Rt 8.46 min, 98.7%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 379.00 (M+H), Rt 1.87 min; Column: X-select CSH C18 (3*50) mm, 2.5 μm. ¹H NMR (400 MHz, DMSO-d6) δ_{H} 9.38-9.32 (m, 1H), 7.86-7.80 (m, 2H), 7.64-7.60 (m, 1H), 7.59 (d, 1H), 7.44-7.36 (m, 2H), 4.49 (d, 2H), 2.51 (s, 3H).

### Example 6. Synthesis of (R)—N-(1-(4-chlorophenyl)ethyl)-5-(N-methylsulfamoyl)thiophene-2-carboxamide (Compound 4)

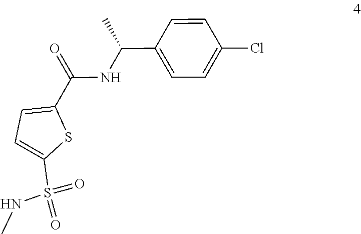 [SMILES: CNS(=O)(=O)c1ccc(C(=O)N[C@H](C)c2ccc(Cl)cc2)s1]

Compound 4 was synthesized according to the procedure described in Example 2. Yield: 80 mg, 0.208 mmol (from 200 mg of a3). HPLC: Rt 8.22 min, 93.5% Column: X-Select CSH C18 (4.6×150) mm, 5 μm; Mobile phase: A: 0.1% Formic acid in water:ACN (95:05), B: ACN; Flow Rate: 1.0 mL/min. LCMS: 359.05 (M+H), Rt 1.82 min Column: X-select CSH C18 (3*50) mm, 2.5 μm. ¹H NMR (400 MHz, DMSO-d6) δ_{H} 9.12 (d, 1H), 7.90 (d, 1H), 7.85-7.78 (m, 1H), 7.58 (d, 1H), 7.42-7.38 (m, 4H), 5.12-5.08 (m, 1H), 1.47 (d, 3H), 3H merged in solvent peak.

### Example 7. Synthesis of (S)—N-(1-(4-chlorophenyl)ethyl)-5-(N-methylsulfamoyl)thiophene-2-carboxamide (Compound 5)

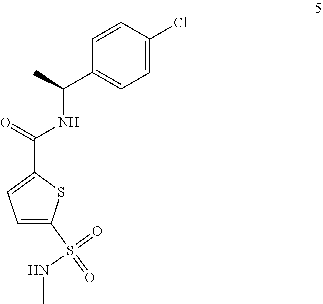 [SMILES: CNS(=O)(=O)c1ccc(C(=O)N[C@@H](C)c2ccc(Cl)cc2)s1]

Compound 5 was synthesized according to the procedure described in Example 2. Yield: 92.8 mg, 0.257 mmol (from 200 mg of a3). HPLC: Rt 8.04 min, 99.7%; Column: X-Select CSH C18 (4.6×150) mm, 3.5 μm; Mobile phase: A: 0.1% Formic acid in water:ACN (95:05), B: ACN; Flow Rate: 1.0 mL/min; LCM: 358.95 (M+H), Rt 1.83 min Column: X-select CSH C18 (3*50) mm, 2.5 μm. ¹H NMR (400 MHz, DMSO-d6) δ_{H} 9.13 (d, 1H), 7.90 (d, 1H), 7.80-7.72 (m, 1H), 7.59 (d, 1H), 7.42-7.36 (m, 4H), 5.12-5.06 (m, 1H), 2.51 (s, 3H), 1.47 (d, 3H).

### Example 8. Synthesis of N-(2,4-dichlorobenzyl)-5-(methylsulfonyl)thiophene-2-carboxamide (Compound 6)

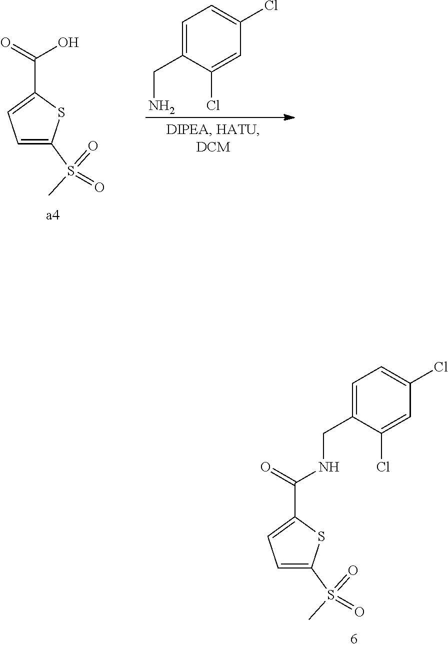 [SMILES: CS(=O)(=O)c1ccc(C(=O)O)s1 ; NCc1ccc(Cl)cc1Cl ; CS(=O)(=O)c1ccc(C(=O)NCc2ccc(Cl)cc2Cl)s1]

5-(methylsulfonyl)thiophene-2-carboxylic acid (a4) was synthesized according to the protocol described in WO 2000/058277. Following the general procedure in Example 2, Compound 6 was afforded as a solid (57.5 mg, 0.157 mmol (from 60 mg of a4)). HPLC: Rt 8.48 min, 99.6%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 364.00 (M+H), Rt 1.97 min; Column: X-select CSH C18 (3*50) mm, 2.5 μm. ¹H NMR (400 MHz, DMSO-d6) δ_{H} 9.42 (t, 1H), 7.90 (d, 1H), 7.84 (d, 1H), 7.64 (s, 1H), 7.46-7.38 (m, 2H), 4.52 (d, 2H), 3.39 (s, 3H).

### Example 9. Synthesis of N-(4-chlorobenzyl)-5-(N-methylsulfamoyl)thiophene-2-carboxamide (Compound 7)

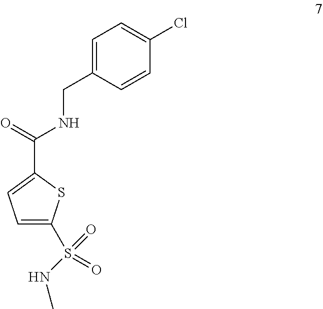 [SMILES: CNS(=O)(=O)c1ccc(C(=O)NCc2ccc(Cl)cc2)s1]

Compound 7 was synthesized according to the procedure described in Example 2. Yield: 97.1 mg, 0.275 mmol (from 200 mg of a3). HPLC: Rt 7.99 min, 98.0%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 344.95 (M+H), Rt 1.95 min Column: X-select CSH C18 (3*50) mm, 2.5 μm. ¹H NMR (400 MHz, DMSO-d6) δ_{H} 9.37 (t, 1H), 7.84-7.80 (m, 2H), 7.59 (d, 1H), 7.40 (d, 2H), 7.33 (d, 2H), 4.45 (d, 2H), 3H merged in solvent peak.

### Example 10. Synthesis of N-(4-chloro-2-(trifluoromethyl)benzyl)-5-(N-methylsulfamoyl)thiophene-2-carboxamide (Compound 8)

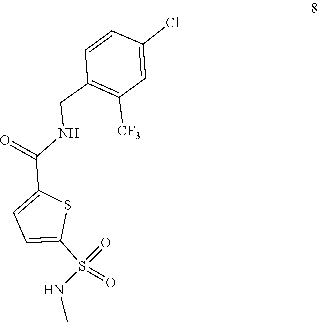 [SMILES: CNS(=O)(=O)c1ccc(C(=O)NCc2ccc(Cl)cc2C(F)(F)F)s1]

Compound 8 was synthesized according to the procedure described in Example 2. Yield: 60 mg, 0.143 mmol, 32% yield as a solid (from 100 mg of a3). HPLC: Rt 8.66 min, 98.1%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 413.05 (M+H), Rt 2.02 min Column: X-select CSH C18 (3*50) mm, 2.5 μm. ¹H NMR (400 MHz, DMSO-d6) δ_{H} 9.48-9.40 (m, 1H), 7.90-7.80 (m, 3H), 7.76 (d, 1H), 7.62 (d, 1H), 7.56 (d, 1H), 4.61 (d, 2H), 2.53 (s, 3H).

### Example 11. Synthesis of N-(4-chloro-2-methylbenzyl)-5-(N-methylsulfamoyl)thiophene-2-carboxamide (Compound 9)

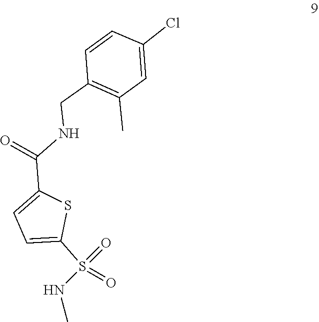 [SMILES: CNS(=O)(=O)c1ccc(C(=O)NCc2ccc(Cl)cc2C)s1]

Compound 9 was synthesized according to the procedure described in Example 2. Yield: 40.0 mg, 0.110 mmol (from 100 mg of a3). HPLC: Rt 8.33 min, 98.5%; Column: X-Select CSH C18 (4.6×150) mm, 5 μm; Mobile phase: A: 0.1% Formic acid in water:ACN (95:05), B: ACN; Flow Rate: 1.0 mL/min. LCM: 358.90 (M+H), Rt 1.91 min Column: X-select CSH C18 (3*50) mm, 2.5 μm. ¹H NMR (400 MHz, DMSO-d6) δ_{H} 9.23 (t, 1H), 7.86-7.82 (m, 2H), 7.59 (d, 1H), 7.28-7.20 (m, 3H), 4.40-4.30 (m, 2H), 2.45 (s, 3H), 3H merged in solvent peak.

### Example 12. Synthesis of N-(4-chloro-2-fluorobenzyl)-5-(N-methylsulfamoyl)thiophene-2-carboxamide (Compound 10)

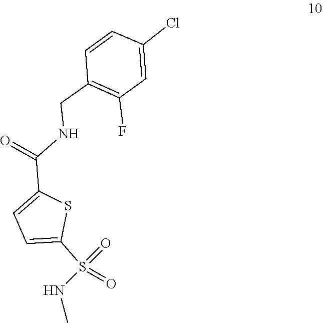 [SMILES: CNS(=O)(=O)c1ccc(C(=O)NCc2ccc(Cl)cc2F)s1]

Compound 10 was synthesized according to the procedure described in Example 2. Yield: 35 mg, 0.095 mmol (from 100 mg of a3). HPLC: Rt 7.94 min, 98.4% Column: X-Select CSH C18 (4.6×150) mm, 5 μm; Mobile phase: A: 0.1% Formic acid in water:ACN (95:05), B: ACN; Flow Rate: 1.0 mL/min. LCMS: 362.85 (M+H), Rt 1.89 min Column: X-select CSH C18 (3*50) mm, 2.5 μm. ¹H NMR (400 MHz, DMSO-d6) δ_{H} 9.35 (t, 1H), 7.85-7.80 (m, 2H), 7.60-7.58 (m, 1H), 7.46-7.36 (m, 2H), 7.28 (d, 1H), 4.47 (d, 2H), 3H merged in solvent peak.

### Example 13. Synthesis of N-(2-bromo-4-chlorobenzyl)-5-(N-methylsulfamoyl)thiophene-2-carboxamide (Compound 11)

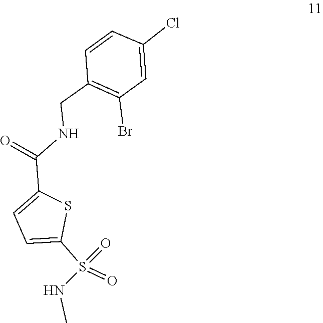 [SMILES: CNS(=O)(=O)c1ccc(C(=O)NCc2ccc(Cl)cc2Br)s1]

Compound 11 was synthesized according to the procedure described in Example 2. Yield: 35 mg, 0.082 mmol (from 100 mg of a3). HPLC: Rt 7.08 min, 97.4%; Column: X-Bridge C18 (4.6×150) mm, 5 μm; Mobile phase: A: 0.1% NH₃ in water: B: ACN; Flow Rate: 1.2 mL/min. LCMS: 424.95 (M+3), Rt 1.97 min; Column: X-select CSH C18 (3*50) mm, 2.5 μm. ¹H NMR (400 MHz, DMSO-d6) δ_{H} 9.38 (d, 1H), 7.86 (d, 1H), 7.84-7.76 (m, 2H), 7.61 (d, 1H), 7.50-7.45 (m, 1H), 7.37 (d, 1H), 4.47 (d, 2H), 2.52-2.46 (m, 3H).

### Example 14. Synthesis of N-(4-chloro-2-cyclopropylbenzyl)-5-(N-methylsulfamoyl)thiophene-2-carboxamide (Compound 12)

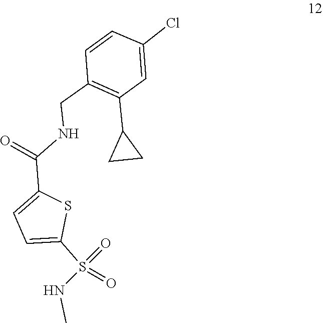 [SMILES: CNS(=O)(=O)c1ccc(C(=O)NCc2ccc(Cl)cc2C2CC2)s1]

Compound 12 was synthesized according to the procedure described in Example 2. Yield: 45 mg, 0.111 mmol (from 100 mg of a3). HPLC: Rt 8.54 min, 95.0%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 384.7 (M+H), Rt 1.90 min; Column: X-select CSH C18 (3*50) mm, 2.5 μm. ¹H NMR (400 MHz, DMSO-d6) δ_{H} 9.26 (t, 1H), 7.85 (d, 1H), 7.82-7.78 (m, 1H), 7.59 (d, 1H), 7.28-7.20 (m, 2H), 7.04-7.00 (m, 1H), 4.61 (d, 2H), 2.52 (s, 3H), 2.06-2.00 (m, 1H), 0.98-0.90 (m, 2H), 0.72-0.067 (m, 2H).

### Example 15. Synthesis of N-(4-chloro-2-methoxybenzyl)-5-(N-methylsulfamoyl)thiophene-2-carboxamide (Compound 13)

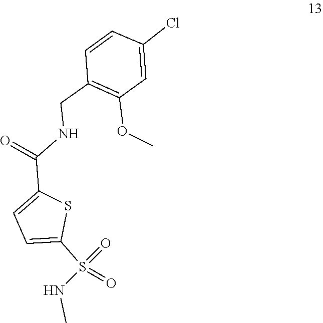 [SMILES: CNS(=O)(=O)c1ccc(C(=O)NCc2ccc(Cl)cc2OC)s1]

Compound 13 was synthesized according to the procedure described in Example 2. Yield: 55 mg, 0.144 mmol (from 100 mg of a3). HPLC: Rt 8.10 min, 98.8%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 375.10 (M+H), Rt 1.83 min Column: X-select CSH C18 (3*50) mm, 2.5 μm. ¹H NMR (400 MHz, DMSO-d6) δ_{H} 9.19 (t, 1H), 7.86-7.80 (m, 2H), 7.59 (d, 1H), 7.19 (d, 1H), 7.08 (s, 1H), 6.98 (d, 1H), 4.38 (d, 2H), 3.85 (s, 3H), 3H merged in solvent peak.

### Example 16. Synthesis of N-(4-chloro-2-hydroxybenzyl)-5-(N-methylsulfamoyl)thiophene-2-carboxamide (Compound 14)

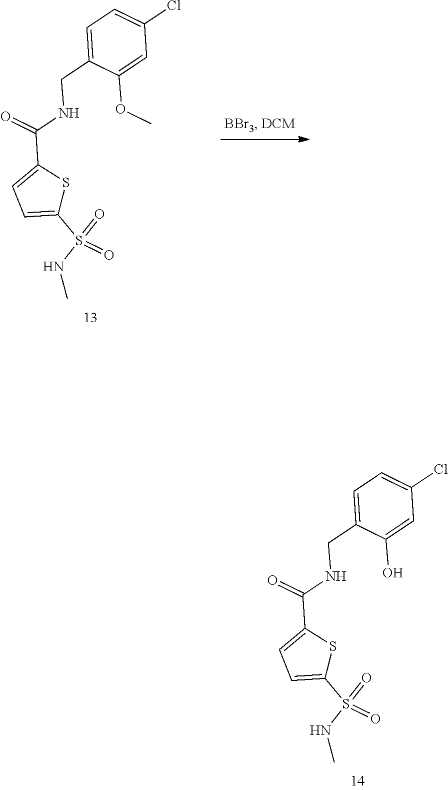 [SMILES: CNS(=O)(=O)c1ccc(C(=O)NCc2ccc(Cl)cc2OC)s1 ; CNS(=O)(=O)c1ccc(C(=O)NCc2ccc(Cl)cc2O)s1]

To a stirred solution of Compound 13 (100 mg, 0.2700 mmol) in DCM was added BBr₃ (1 M in DCM, 0.8 mL, 0.8 mmol). The reaction mixture was stirred at 0° C. for 30 min. The reaction was quenched with methanol (2 mL) and the organic layer was concentrated under reduced pressure. The crude compound was purified by column chromatography using 100-200 silica and at 30-80% EtOAc/Hexane as an eluent to afford 14 (40 mg, 0.108 mmol, 41% yield) as a solid. HPLC: Rt 7.79 min, 97%; Column: X-Select CSH C18 (4.6×150) mm, 5 μm; Mobile phase: A: 0.1% Formic acid in water:ACN (95:05), B: ACN; Flow Rate: 1.0 mL/min. LCMS: 360.95 (M+H), Rt 1.95 min; Column: X-select CSH C18 (3*50) mm, 2.5 μm. ¹H NMR (400 MHz, DMSO-d6) δ_{H} 10.13 (s, 1H), 9.25-9.15 (m, 1H), 7.86-7.80 (m, 2H), 7.59-7.56 (m, 1H), 7.14 (d, 1H), 6.86-6.80 (m, 2H), 4.37 (d, 2H), 3H merged in solvent peak.

### Example 17. Synthesis of 5-(Cyclopropylsulfonyl)-N-(2,4-dichlorobenzyl)thiophene-2-carboxamide (Compound 15)

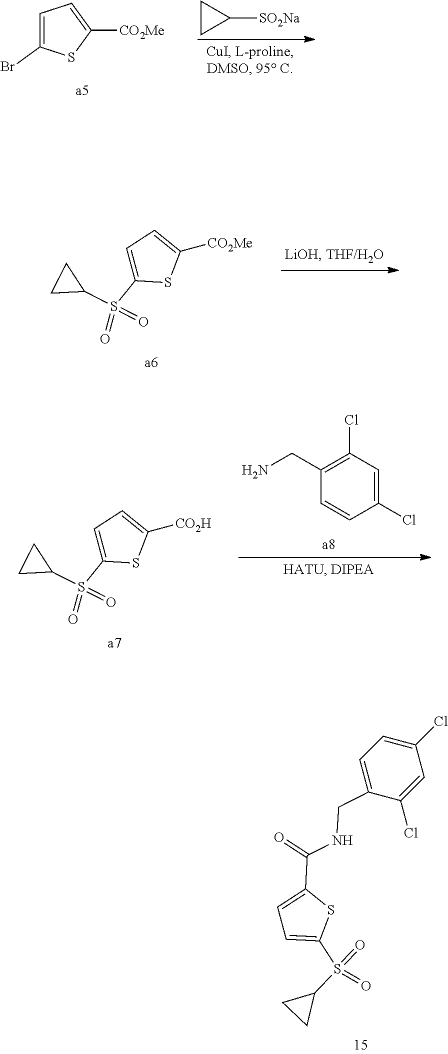

### Synthesis of Methyl 5-(cyclopropylsulfonyl)thiophene-2-carboxylate (a6)

To a stirred solution of a5 (1.5 g, 6.79 mmol) and sodium cyclopropanesulfinate (1.3 g, 10.18 mmol) in DMSO (20 mL) was added copper iodide (0.13 g, 0.68 mmol), L-proline (0.16 g, 1.36 mmol) and sodium hydroxide (0.054 g, 1.35 mmol) at RT. The reaction mixture was stirred at 95° C. for 16 h. The reaction mixture was diluted with water (50 mL) and extracted with EtOAc (2×50 mL). The organic layer was separated, dried over anhydrous Na₂SO₄, filtered and evaporated under reduced pressure to give the crude product. The crude product was purified by silica gel column chromatography using 20-40% EtOAc/Hexane as eluent to afford a6 (0.4 g, 1.54 mmol, 23% yield) as a solid.

### Synthesis of 5-(cyclopropylsulfonyl)thiophene-2-carboxylic acid (a7)

To a stirred solution of a6 (0.4 g, 1.62 mmol) in THF:water (10 mL:3 mL) was added LiOH·H₂O (0.102 g, 2.44 mmol) at RT. The reaction mixture was stirred at RT for 2 h. The reaction mixture was diluted with water (10 mL) and extracted with EtOAc (2×20 mL). The organic layer was separated and the aqueous layer was acidified with 1N HCl. The precipitated solid was collected by filtration and dried under reduced pressure to afford a7 (0.23 g, 0.95 mmol, 58% yield) as a solid.

### Synthesis of 5-(cyclopropylsulfonyl)-N-(2,4-dichlorobenzyl)thiophene-2-carboxamide (Compound 15)

To a stirred solution of a7 (105.45 mg, 0.45 mmol) and a8 (0.06 mL, 0.45 mmol) in DCM (10 mL) at 0° C. HATU (207.14 mg, 0.54 mmol) and DIPEA (0.16 mL, 0.91 mmol) was added and the resulting reaction mixture was stirred at RT for 16 h. The reaction mixture was diluted with water (10 mL) and extracted with DCM (2×50 mL). The combined organic layer was separated, dried over anhydrous sodium sulphate, filtered and concentrated under reduced pressure to afford the crude compound. The crude compound was purified by silica gel column chromatography using 30-80% EtOAc/hexane as eluent to afford 15 (30 mg, 0.077 mmol, 17% yield). HPLC: Rt 8.64 min, 99.81%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 391.8 (M+2), Rt 1.967 min, Column: X-select CSH C18 (3*50) mm, 2.5 μm. ¹H NMR (400 MHz, DMSO-d6) δ 9.42 (t, 1H), 7.91 (d, 1H), 7.81 (d, 1H), 7.67-7.61 (m, 1H), 7.48-7.37 (m, 2H), 4.52 (d, 2H), 3.05-2.98 (m, 1H), 1.23-1.08 (m, 4H).

### Example 18. Synthesis of N-(2,4-dichlorobenzyl)-5-(ethylsulfonyl)thiophene-2-carboxamide (Compound 16)

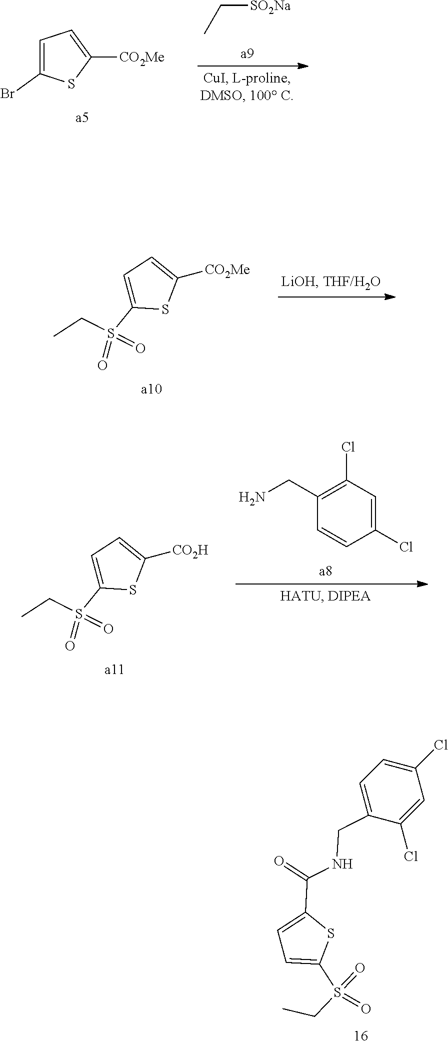

### Synthesis of methyl 5-(ethylsulfonyl)thiophene-2-carboxylate (a10)

To a stirred solution of a5 (1 g, 4.52 mmol) in DMSO (10 mL) was added a9 (630.26 mg, 5.43 mmol), copper iodide (85.95 mg, 0.45 mmol), sodium hydroxide (36.19 mg, 0.90 mmol) and L-proline (104.16 mg, 0.90 mmol) at RT and stirred at 95° C. for 16 h. The reaction mixture was diluted with water (100 mL) and extracted with EtOAc (5×25 mL). The combined organic layer thus obtained was dried over Na₂SO₄ and evaporated to obtain crude compound. The crude compound was purified by column chromatography in 100-200 silica at 8-10% EtOAc/Hexane eluent to give a10 (300 mg, 1.21 mmol, 26% yield) as a solid.

### Synthesis of 5-(ethylsulfonyl)thiophene-2-carboxylic acid (a11)

To a stirred solution of a10 (300 mg, 1.28 mmol) in THE (5 mL) was added lithium hydroxide (46 mg, 1.92 mmol) in water (1 mL) at 0° C. and stirred at RT for 2 h. The reaction mixture was concentrated to get the crude product. The crude product thus obtained was diluted with cold water (10 mL), acidified with 2N HCl aq. solution up to pH-4 and extracted with DCM (3×15 mL). The combined organic layer was separated and dried over Na₂SO₄ to give a11 (220 mg, 0.60 mmol, 46% yield, 60% purity) as a solid.

### Synthesis of N-(2,4-dichlorobenzyl)-5-(ethylsulfonyl)thiophene-2-carboxamide (Compound 16)

To a stirred solution of a11 (100 mg, 0.45 mmol) and a8 (0.06 mL, 0.45 mmol) in DCM (10 mL) were added HATU (207.14 mg, 0.54 mmol) and DIPEA (0.16 mL, 0.91 mmol) at RT. The reaction mixture was stirred at RT for 2 hr. The reaction mixture was quenched with water (10 mL) and extracted with DCM (2×50 mL). The organic layer was separated, dried over anhydrous Na₂SO₄, filtered and concentrated under reduced pressure. The crude reaction mass was purified by silica gel column chromatography using 30-80% EtOAc/Hexane as eluent to give 16 (30 mg, 0.0791 mmol, 17% yield) as a solid. HPLC: Rt 8.48 min, 99.70%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 379.7 (M+2), Rt 1.936 min, Column: X-select CSH C18 (3*50) mm, 2.5 μm. ¹H NMR (400 MHz, DMSO-d6) δ 9.42 (t, 1H), 7.93 (d, 1H), 7.81 (d, 1H), 7.67-7.61 (m, 1H), 7.48-7.37 (m, 2H), 4.52 (d, 2H), 3.44 (q, 2H), 1.18 (t, 3H).

### Example 19. Synthesis of N-(4-cyanobenzyl)-5-(N-methylsulfamoyl)thiophene-2-carboxamide (Compound 17) and Synthesis of N-(3-chlorobenzyl)-5-(N-methylsulfamoyl)thiophene-2-carboxamide (Compound 18)

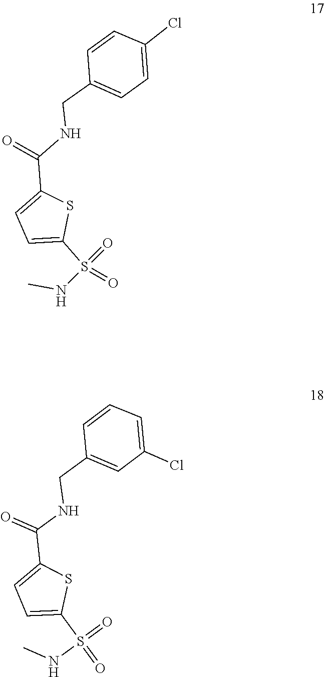 [SMILES: CNS(=O)(=O)c1ccc(C(=O)NCc2ccc(Cl)cc2)s1 ; CNS(=O)(=O)c1ccc(C(=O)NCc2cccc(Cl)c2)s1]

Compounds 17 and 18 were made following synthetic methods described in Example 2. Compound 17: Yield: 20 mg, 0.0586 mmol, 13%, HPLC: Rt 7.33 min, 98.28%, Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 335.90 (M+H), Rt 1.909 min, Column: X-select CSH C18 (3*50) mm, 2.5 μm. ¹H NMR (400 MHz, DMSO-d6) δ 9.44 (t, 1H), 7.84-7.79 (m, 4H), 7.60 (d, 1H), 7.51 (d, 2H), 4.55 (d, 2H), 3H merged in solvent peak. Chiral method: Rt: 9.329 min, 99.47%; column: YMC CHIRAL ART CELLULOSE-SC (250×4.6 mm, 5u), Mobile Phase: A) n-Hexane+0.1% TFA, B) DCM:MeOH (50:50), Isocratic: 35% B; Wavelength: 267 nm, Flow: 1.0 mL/min. Compound 18: Yield: 25 mg, 0.072 mmol, 16% %, HPLC: Rt 8.075 min, 98.91%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 344.85 (M+H), Rt 2.088 min, Column: X-select CSH C18 (3*50) mm, 2.5 μm. ¹H NMR (400 MHz, DMSO-d6) δ 9.40-9.35 (m, 1H), 7.84-7.80 (m, 2H), 7.57 (d, 1H), 7.38-7.26 (m, 4H), 4.46 (d, 2H), 3H merged in solvent peak. Chiral method: Rt: 6.757 min, 99.69%; column: YMC CHIRAL ART CELLULOSE-SC (250×4.6 mm, 5u), Mobile Phase: A) n-Hexane+0.1% TFA, B) DCM:MeOH (50:50), Isocratic: 35% B; Wavelength: 268 nm, Flow: 1.0 mL/min.

### Example 20. Synthesis of N-(4-isopropylbenzyl)-5-(N-methylsulfamoyl)thiophene-2-carboxamide (Compound 19)

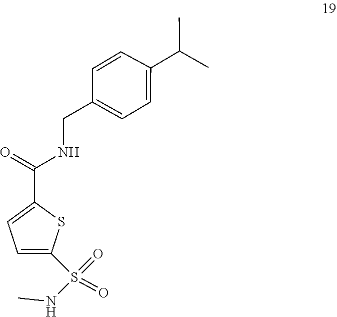 [SMILES: CNS(=O)(=O)c1ccc(C(=O)NCc2ccc(C(C)C)cc2)s1]

To a stirred solution of (4-isopropylphenyl)methanamine (80.94 mg, 0.54 mmol) and a3 (100 mg, 0.45 mmol) in DCM (4 mL), DIPEA (0.24 mL, 1.36 mmol) and HATU (257.78 mg, 0.68 mmol) was added. The reaction mixture was stirred at RT for 3 h. The reaction mixture was diluted with water and extracted with DCM. The organic layer was separated, dried over anhydrous sodium sulphate and concentration under reduced pressure. The crude compound was purified by prep. HPLC to afford 10 (15 mg, 0.04 mmol, 9% yield,) as a solid. HPLC: Rt 8.321 min, 95.32%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 352.9 (M+H), Rt 1.985 min, Column: X-select CSH C18 (3*50) mm, 2.5 μm. ¹H NMR (400 MHz, DMSO-d6) δ 9.32-9.25 (m, 1H), 7.85-7.75 (m, 2H), 7.58-7.56 (m, 1H), 7.30-7.15 (m, 4H), 4.46 (d, 2H), 2.88-2.83 (m, 1H), 1.18 (d, 6H), 3H merged in solvent peak.

### Example 21. Synthesis of N-(4-chloro-3-fluorobenzyl)-5-(N-methylsulfamoyl)thiophene-2-carboxamide (Compound 20)

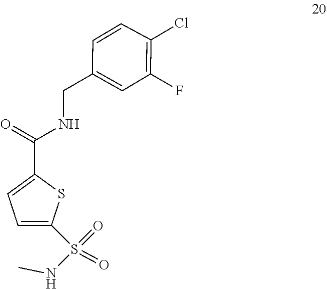 [SMILES: CNS(=O)(=O)c1ccc(C(=O)NCc2ccc(Cl)c(F)c2)s1]

To a stirred solution of a3 (100 mg, 0.45 mmol) and corresponding amine (86.56 mg, 0.54 mmol) in DCM (5 mL) was added DIPEA (0.16 mL, 0.90 mmol) followed by HATU (206.23 mg, 0.54 mmol) at 0° C. and the resulting reaction mixture was stirred at RT for 16 h. The reaction mixture was diluted with water and extracted with ethyl acetate. The combined organic layer was separated, dried over anhydrous sodium sulphate, filtered and concentrated under reduced pressure to afford the crude compound. The crude compound was purified by silica gel column chromatography using 30-80% EtOAc/Hexane as eluent to afford 20 (48 mg, 0.13 mmol, 29%) as a solid. HPLC: Rt 8.065 min, 98.75%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 362.8 (M+H), Rt 1.923 min, Column: X-Select CSH C18 (4.6×150) mm, 2.5 μm; ¹H NMR (400 MHz, DMSO-d6) δ 9.42-9.35 (m, 1H), 7.824-7.80 (m, 2H), 7.62-7.51 (m, 2H), 7.35 (d, 1H), 7.19 (d, 1H), 4.47 (d, 2H), 3H merged in solvent peak.

### Example 22. Synthesis of N-(4-bromobenzyl)-5-(N-methylsulfamoyl)thiophene-2-carboxamide (Compound 21)

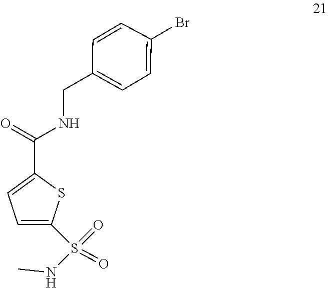 [SMILES: CNS(=O)(=O)c1ccc(C(=O)NCc2ccc(Br)cc2)s1]

To a stirred solution of a3 (100.mg, 0.45 mmol) and corresponding amine (100.91 mg, 0.54 mmol) in DCM (5 mL) were added DIPEA (0.16 mL, 0.90 mmol) followed by HATU (206.23 mg, 0.54 mmol) at 0° C. and the resulting reaction mixture was stirred at RT for 16 h. The reaction mixture was diluted with water and extracted with ethyl acetate. The combined organic layer was separated, dried over anhydrous sodium sulphate, filtered and concentrated under reduced pressure to afford the crude compound. The crude compound was purified by silica gel column chromatography using 30-80% EtOAc/Hexane as eluent to afford 21 (20 mg, 0.05 mmol, 11%) as a solid. HPLC: Rt 7.822 min, 96.13%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 388.8 (M+H), Rt 2.079 min, Column: X-Select CSH C18 (4.6×150) mm, 2.5 μm. ¹H NMR (400 MHz, DMSO-d6) δ 9.38-9.35 (m, 1H), 7.82-7.78 (m, 2H), 7.61-7.58 (m, 1H), 7.53 (d, 2H), 7.28 (d, 2H), 4.43 (d, 2H), 3H merged in solvent peak.

### Example 23. Synthesis of N-(4-methylbenzyl)-5-(N-methylsulfamoyl)thiophene-2-carboxamide (Compound 22)

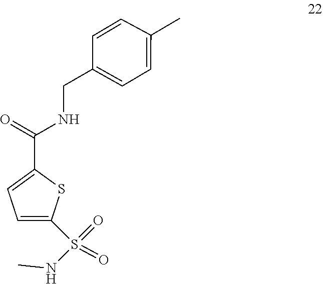 [SMILES: CNS(=O)(=O)c1ccc(C(=O)NCc2ccc(C)cc2)s1]

To a stirred solution of a3 (100 mg, 0.45 mmol) and corresponding amine (65.7 mg, 0.54 mmol) in DCM (5 mL) was added DIPEA (0.16 mL, 0.90 mmol) followed by HATU (206.2 mg, 0.54 mmol) at 0° C. and the resulting reaction mixture was stirred at RT for 16 h. The reaction mixture was diluted with water and extracted with ethyl acetate. The combined organic layer was separated, dried over anhydrous sodium sulphate, filtered and concentrated under reduced pressure to afford the crude compound. The crude compound was purified by silica gel column chromatography using 30-80% EtOAc/Hexane as eluent to afford 22 (30 mg, 0.09 mmol, 20%) as a solid. HPLC: Rt 7.588 min, 99.01%; Column: X-Select CSH C18 (4.6×150) mm, 3.5 μm; Mobile phase: A: 0.1% Formic acid in water:ACN (95:05), B: ACN; Flow Rate: 1.0 mL/min. LCMS: 324.9 (M+H), Rt 1.877 min, Column: X-Select CSH C18 (4.6×150) mm, 2.5 μm. ¹H NMR (400 MHz, DMSO-d6) δ 9.33-9.25 (m, 1H), 7.81 (d, 2H), 7.57 (d, 1H), 7.23-7.11 (m, 4H), 4.41 (d, 2H), 2.28 (s, 3H), 3H merged in solvent peak.

### Example 24. Synthesis of 5-(methylsulfonyl)-N-(4-(pyrrolidin-1-yl)benzyl)thiophene-2-carboxamide (Compound 23)

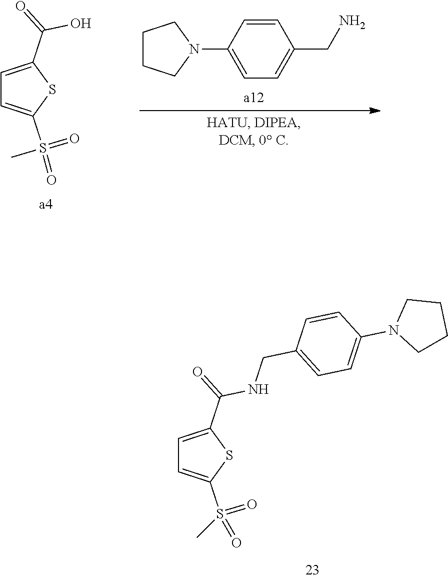 [SMILES: CS(=O)(=O)c1ccc(C(=O)O)s1 ; NCc1ccc(N2CCCC2)cc1 ; CS(=O)(=O)c1ccc(C(=O)NCc2ccc(N3CCCC3)cc2)s1]

To a stirred reaction mixture of 5-methylsulfonylthiophene-2-carboxylic acid (200 mg, 0.9697 mmol) and a12 (205.11 mg, 1.1637 mmol) in DCM (20 mL) was added HATU (553.09 g, 1.4546 mmol) followed by DIPEA (376.02 mg, 2.9092 mmol) at 0° C., then stirring was continued further for 1 h at 0° C. The reaction mixture was concentrated under reduced pressure, and then diluted by adding water (10.0 mL) and then the reaction mixture was extracted with EtOAc (2×25 mL), the combined extracts were dried over anhydrous Na₂SO₄, filtered, concentrated under reduced pressure to obtain the crude residue (220 mg) as a viscous liquid. The crude material was purified by Combi-Flash column chromatography (100-200 silica gel), eluting 0-40% EtOAc in hexanes to afford 23 (75.4 mg, 0.2012 mmol, 20% yield) as a solid. LCMS: 365.1 (M+H), Rₜ=2.206 min, Column: X-Bridge BEH C-18 (3.0×50 mm, 2.5 μm); Mobile Phase: A: 0.025% FA in Water, B: ACN; T/B %: 0.01/2, 0.2/2, 2/98, 3/98, 3.2/2, 4/2; Flow rate: 1.2 ml/min (Gradient); Column Oven temperature: 50° C. HPLC: Rₜ=6.138 min, 97.26%; Column: XSELECT CSH C18 (150×4.6 mm, 3.5); Mobile Phase-A: 0.05% TFA:ACETONITRILE (95:05); Mobile Phase-B: ACETONITRILE:0.05% TFA (95:05); Program: T/B %: 0.01/10, 12/90, 16/90; Flow: 1.0 mL/min; Diluent: ACN:WATER. ¹H NMR (400 MHz, DMSO-d6) δ 9.23 (t, 1H), 7.84 (d, 1H), 7.79 (d, 1H), 7.11 (d, 2H), 6.49 (d, 2H), 4.33 (d, 2H), 3.37 (s, 3H), 3.18 (t, 4H), 1.98-1.89 (m, 4H).

### Example 25. Synthesis of N-(4-isopropylbenzyl)-5-(methylsulfonyl)thiophene-2-carboxamide (Compound 24)

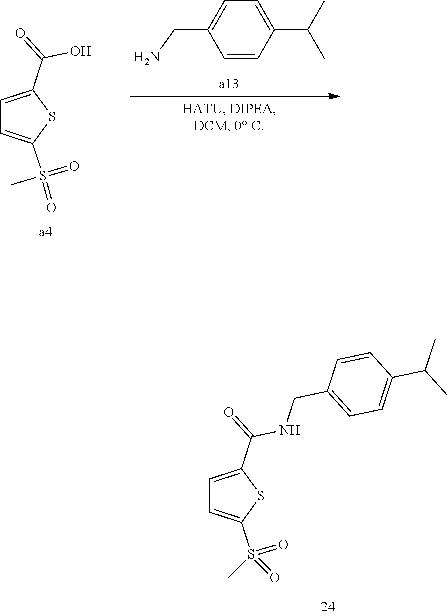 [SMILES: CS(=O)(=O)c1ccc(C(=O)O)s1 ; CC(C)c1ccc(CN)cc1 ; CC(C)c1ccc(CNC(=O)c2ccc(S(C)(=O)=O)s2)cc1]

To a stirred reaction mixture of 5-methylsulfonylthiophene-2-carboxylic acid (200 mg, 0.9697 mmol) and a13 (217.07 mg, 1.4546 mmol) in DCM (25 mL) was added HATU (553.09 mg, 1.4546 mmol) followed by DIPEA (376.02 mg, 2.9092 mmol) at 0° C., then stirring was continued further for 1 h at 0° C. The reaction mixture was concentrated under reduced pressure, and then diluted by adding water (10.0 mL) and then the reaction mixture was extracted with EtOAc (2×25 mL), the combined extracts were dried over anhydrous Na₂SO₄, filtered, concentrated under reduced pressure to obtain the crude residue (220 mg) as a viscous liquid. The obtained crude was purified by Combi-Flash column chromatography (100-200 silica gel) by eluting 0-40% EtOAc in hexanes to afford 24 (183.1 mg, 0.54 mmol, 55% yield) as a solid. LCMS: 338.1 (M+H), Rₜ=2.335 min, Column: X-Bridge BEH C-18 (3.0×50 mm, 2.5 μm); Mobile Phase: A: 0.025% FA in Water, B: ACN; T/B %: 0.01/2, 0.2/2, 2/98, 3/98, 3.2/2, 4/2; Flow rate: 1.2 ml/min(Gradient); Column Oven temperature: 50° C. HPLC: Rₜ=10.841 min, 99.22%; Column: XSELECT CSH C18 (150×4.6 mm, 3.5); Mobile Phase-A: 0.05% TFA:ACETONITRILE (95:05); Mobile Phase-B: ACETONITRILE:0.05% TFA (95:05); Program: T/B %: 0.01/10, 12/90, 16/90; Flow: 1.0 mL/min; Diluent: ACN:WATER. ¹H NMR (400 MHz, DMSO-d6) δ 9.34 (t, 1H), 7.86 (d, 1H), 7.81 (d, 1H), 7.27-7.18 (m, 4H), 4.43 (d, 2H), 3.38 (s, 3H), 2.92-2.80 (m, 1H), 1.18 (d, 6H).

### Example 26. Synthesis of N-(4-isopropoxybenzyl)-5-(methylsulfonyl)thiophene-2-carboxamide (Compound 25)

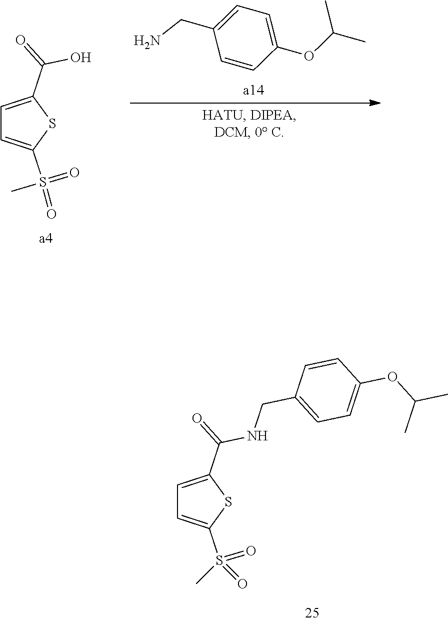 [SMILES: CS(=O)(=O)c1ccc(C(=O)O)s1 ; CC(C)Oc1ccc(CN)cc1 ; CC(C)Oc1ccc(CNC(=O)c2ccc(S(C)(=O)=O)s2)cc1]

To a stirred reaction mixture of 5-methylsulfonylthiophene-2-carboxylic acid (200 mg, 0.9697 mmol) and a14 (240.35 mg, 1.4546 mmol) in DCM (5.00 mL) was added HATU (553.09 mg, 1.4546 mmol) followed by DIPEA (376.02 mg, 2.9092 mmol) at 0° C., then stirring was continued further for 1 h at 0° C. The reaction mixture was concentrated under reduced pressure, and then diluted by adding water (10.0 mL) and then the reaction mixture was extracted with EtOAc (2×25 mL), the combined extracts were dried over anhydrous Na₂SO₄, filtered, concentrated under reduced pressure to obtain the crude residue (220 mg) as a colorless viscous liquid. The obtained crude was purified by Combi-Flash column chromatography (100-200 silica gel) by eluting 0-40% EtOAc in hexanes to afford 25 (161.19 mg, 0.4537 mmol, 46% yield) as a solid. LCMS: 354.1 (M+H), Rₜ=2.184 min, Column: X-Bridge BEH C-18 (3.0×50 mm, 2.5 μm); Mobile Phase: A: 0.025% FA in Water, B: ACN; T/B %: 0.01/2, 0.2/2, 2/98, 3/98, 3.2/2, 4/2; Flow rate: 1.2 ml/min(Gradient). Column Oven temperature: 50° C. HPLC: Rₜ=10.56 min, 99.48%; Column; X SELECT CSH C18 (150×4.6 mm, 3.5μ); Mobile Phase A; 5 mM AMMONIUM BICARBONATE; Mobile Phase B: ACETONITRILE; Program: T/B %: 0.01/20, 5/80, 12/90, 16/90; Flow: 1.0 mL/min; Diluent: ACN:WATER. ¹H NMR (400 MHz, DMSO-d6) δ 9.31 (t, 1H), 7.85 (d, 1H), 7.80 (d, 1H), 7.21 (d, 2H), 6.87 (d, 2H), 4.61-4.52 (m, 1H), 4.39 (d, 2H), 3.37 (s, 3H), 1.24 (d, 6H).

### Example 27. Synthesis of N-(4-cyclopropylbenzyl)-5-(N-methylsulfamoyl)thiophene-2-carboxamide (Compound 26)

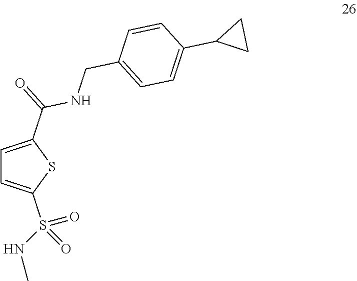 [SMILES: CNS(=O)(=O)c1ccc(C(=O)NCc2ccc(C3CC3)cc2)s1]

To a stirred solution of a3 (200 mg, 0.9040 mmol) in DMF (10 mL) was added DIPEA (323 mg, 2.499 mmol), HATU (443 mg, 1.1651 mmol) followed by corresponding amine (244 mg, 1.6574 mmol) at 0° C. The reaction mixture was stirred at room temperature for 16 h. The reaction mass was quenched with water, extracted with EtOAc (50 mL×2). The combined organic layers were washed with water, brine, dried over anhydrous Na₂SO₄ and concentrated under reduced pressure. The crude product was purified by flash column chromatography using EtOAc in heptane: 0% to 35% to 90% as eluent to afford 26 (52.2 mg, 0.1453 mmol, 16%). LCMS: 349.15 (M−H), Rₜ=2.059 min, Column: X-SELECT CSH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50 C; Gradient program: 0% B to 98% B in 2.0 minute, hold till 3.0 min, at 3.2 min B conc is 0% up to 4.0 min. HPLC: Rₜ=9.09 min, 97.55%; Mobile Phase A: 5 mM Ammonium Bi Carbonate; Mobile Phase B: Acetonitrile; Program: T/B %: 0.01/20, 12/90, 16/90; Flow: 1.0 mL/min; Diluent: WATER:ACN. ¹H NMR (400 MHz, DMSO-d6) δ 9.27 (t, 1H), 7.87-7.74 (m, 2H), 7.62-7.53 (m, 1H), 7.25-7.12 (m, 2H), 7.10-6.97 (m, 2H), 4.45-4.31 (m, 2H), 1.96-1.81 (m, 1H), 0.97-0.81 (m, 2H), 0.67-0.55 (m, 2H), 3H Merged in solvent peak.

### Example 28. Synthesis of N-(4-isopropoxybenzyl)-5-(N-methylsulfamoyl)thiophene-2-carboxamide (Compound 27)

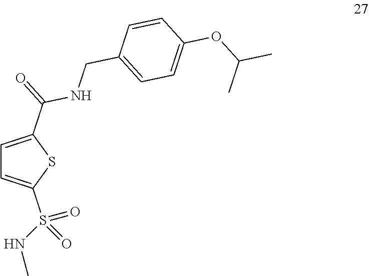 [SMILES: CNS(=O)(=O)c1ccc(C(=O)NCc2ccc(OC(C)C)cc2)s1]

To a stirred solution of a3 (200 mg, 0.9040 mmol) in DMF (15 mL) was added DIPEA (323 mg, 2.499 mmol) and HATU (443 mg, 1.1651 mmol) followed by the corresponding amine (234 mg, 1.4162 mmol) at 0° C. The reaction mixture was stirred at room temperature for 16 h. The reaction mixture was quenched with water, extracted with EtOAc (50 mL×2). The combined organic layers were washed with water, brine, dried over anhydrous Na₂SO₄ and concentrated under reduced pressure. The crude was purified by flash column chromatography using EtOAc in heptane: 0% to 35% to 90% as eluent to afford 27 (41 mg, 0.1112 mmol, 12%). LCMS: 366.8 (M−H), Rₜ=3.214 min, Column: X-Bridge BEH C-18 (3.0×50 mm, 2.5 μm); Mobile Phase: A: 2.5 mM Ammonium Bicarbonate, B: ACN; Gradient T/B %: 0.01/10, 3/90, 5/90, 5.5/10, 6/10; Flow rate: 0.8 ml/min. HPLC: Rₜ=9.17 min, 99.95%; Mobile Phase A: 5 mM Ammonium Bi Carbonate; Mobile Phase B: Acetonitrile; Program: T/B %: 0.01/20, 12/90, 16/90; Flow: 1.0 mL/min; Diluent: WATER:ACN. ¹H NMR (400 MHz, DMSO-d6) δ 9.25 (t, 1H), 7.87-7.72 (m, 2H), 7.61-7.51 (m, 1H), 7.21 (d, 2H), 6.87 (d, 2H), 4.63-4.49 (m, 1H), 4.38 (d, 2H), 1.24 (d, 6H), 3H Merged in solvent peak.

### Example 29. Synthesis of N-(4-(tert-butyl)benzyl)-5-(N-methylsulfamoyl)thiophene-2-carboxamide (Compound 28)

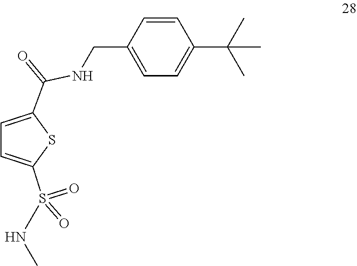 [SMILES: CNCS(=O)(=O)c1ccc(C(=O)NCc2ccc(C(C)(C)C)cc2)s1]

To a stirred solution of a3 (200 mg, 0.9040 mmol) in DMF (10 mL), was added HATU (443 mg, 1.1651 mmol) and DIPEA (323 mg, 2.499 mmol) followed by corresponding amine (244 mg, 1.4945 mmol) at 0° C. The reaction mixture was stirred at room temperature for 16 h. The reaction mixture was quenched with water (10 mL) and extracted with EtOAc (2×50 mL). The combined organic layers were dried over Na₂SO₄ and concentrated under reduced pressure. The crude was purified by flash column chromatography using EtOAc in heptane=0% to 35% to 90% as an eluent to afford 28 (50.3 mg, 0.13 mmol, 15%). LCMS: 365.25 (M−H), Rₜ=2.066 min; Column: X-SELECT CSH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. HPLC: Rₜ=10.160 min, 97.04%; Column: XSELECT CSH C18 (150×4.6 mm, 3.5); Mobile Phase-A: 0.05% TFA:ACETONITRILE (95:05); Mobile Phase-B: ACETONITRILE:0.05% TFA (95:05); Program: T/B %: 0.01/10, 12/90, 16/90; Flow: 1.0 mL/min; Diluent: ACN:WATER. ¹H NMR (400 MHz, DMSO-d6) δ 9.28 (t, 1H), 7.87-7.74 (m, 2H), 7.62-7.53 (m, 1H), 7.36 (d, 2H), 7.24 (d, 2H), 4.46-4.34 (m, 2H), 1.26 (s, 9H), 3H Merged in solvent peak.

### Example 30. Synthesis of 5-(N-methylsulfamoyl)-N-(4-(trifluoromethyl)benzyl)thiophene-2-carboxamide (Compound 29)

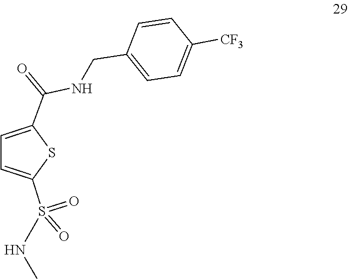 [SMILES: CNCS(=O)(=O)c1ccc(C(=O)NCc2ccc(C(F)(F)F)cc2)s1]

To a stirred solution of a3 (200 mg, 0.9040 mmol) in DMF (15 mL) was added HATU (443 mg, 1.1651 mmol) and DIPEA (323 mg, 2.499 mmol) followed by corresponding amine (274 mg, 1.5644 mmol) at 0° C. The reaction mixture was stirred at room temperature for 16 h. The reaction mixture was quenched with water (10 mL) and extracted with EtOAc (2×50 mL). The combined organic layers were dried over anhydrous Na₂SO₄ and concentrated under reduced pressure. The crude was purified by flash column chromatography using EtOAc in heptane=0% to 35% to 90% as an eluent to afford the title compound 29 (31 mg, 0.0802 mmol, 9%). LCMS: 379.0 (M+H), Rₜ=2.066 min, Column: X-Bridge BEH C-18 (3.0×50 mm, 2.5 μm); Mobile Phase: A: 0.025% FA in Water, B: ACN; T/B %: 0.01/2, 0.2/2, 2/98, 3/98, 3.2/2, 4/2; Flow rate: 1.2 ml/min(Gradient); Column Oven temperature: 50° C. HPLC: Rₜ=8.200 min, 97.94%; Column: XSELECT CSH C18 (150×4.6 mm, 3.5); Mobile Phase-A: 0.05% TFA:ACETONITRILE (95:05); Mobile Phase-B: ACETONITRILE:0.05% TFA (95:05); Program: T/B %: 0.01/10, 12/90, 16/90; Flow: 1.0 mL/min; Diluent: ACN:WATER. ¹H NMR (400 MHz, DMSO-d6) δ 9.43 (t, 1H), 7.82 (s, 2H), 7.71 (d, 2H), 7.60 (s, 1H), 7.54 (d, 2H), 4.60-4.47 (m, 2H), 3H Merged in solvent peak.

### Example 31. Synthesis of 5-(N-methylsulfamoyl)-N-(4-(pyrrolidin-1-yl)benzyl)thiophene-2-carboxamide (Compound 30)

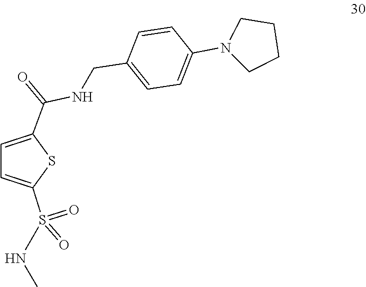 [SMILES: CNCS(=O)(=O)c1ccc(C(=O)NCc2ccc(N3CCCC3)cc2)s1]

To a stirred solution of a3 (200 mg, 0.9040 mmol) in DMF (10 mL), was added HATU (443 mg, 1.1651 mmol) and DIPEA (323 mg, 2.499 mmol) followed by corresponding amine (244 mg, 1.3843 mmol) at 0° C. The reaction mixture was stirred at room temperature for 16 h. The reaction mixture was quenched with water (10 mL) and extracted with EtOAc (2×50 mL). The combined organic layers were dried over Na₂SO₄ and concentrated under reduced pressure. The crude material was purified by flash column chromatography using EtOAc in heptane=0% to 35% to 90% as an eluent to afford 30 (27.6 mg, 0.0802 mmol, 9%). LCMS: 380.2 (M+H), Rₜ=3.389 min, Column: X-Bridge BEH C-18 (3.0×50 mm, 2.5 μm); Mobile Phase: A: 2.5 mM Ammonium Bicarbonate, B: ACN; (Gradient) T/B %: 0.01/10, 3/90, 5/90, 5.5/10, 6/10; Flow rate: 0.8 ml/min. HPLC: Rₜ=9.58 min, 97.12%; Mobile Phase A: 5 mM Ammonium Bi Carbonate; Mobile Phase B: Acetonitrile; Program: T/B %: 0.01/20, 12/90, 16/90; Flow: 1.0 mL/min; Diluent: WATER:ACN. ¹H NMR (400 MHz, DMSO-d₆) 9.16 (t, 1H), 7.79 (d, 2H), 7.56 (d, 1H), 7.11 (d, 2H), 6.49 (d, 2H), 4.32 (d, 2H), 3.14 (t, 4H), 1.93 (t, 4H), 3H Merged in solvent peak.

### Example 32. Synthesis of N-(4-cyclopropylbenzyl)-5-(methylsulfonyl)thiophene-2-carboxamide (Compound 31)

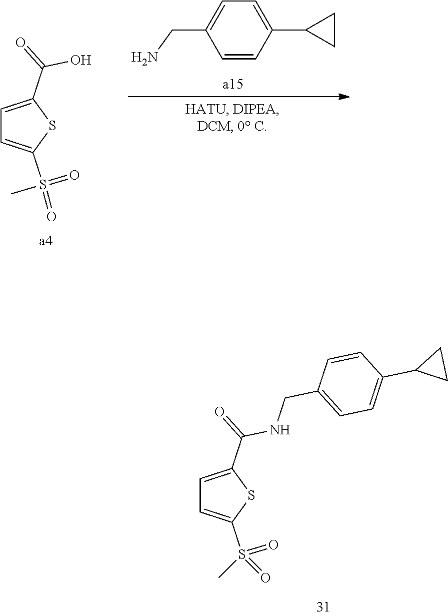 [SMILES: CS(=O)(=O)c1ccc(C(=O)O)s1 ; NCc1ccc(C2CC2)cc1 ; CS(=O)(=O)c1ccc(C(=O)NCc2ccc(C3CC3)cc2)s1]

To a stirred reaction mixture of 5-methylsulfonylthiophene-2-carboxylic acid (200 mg, 0.9697 mmol) and a15 (214.15 mg, 1.4546 mmol) in DCM (20 mL) was added HATU (553.09 mg, 1.4546 mmol) followed by DIPEA (376.02 mL, 2.9092 mmol) at 0° C., then stirring was continued further for 1 h at 0° C. The reaction mixture was concentrated under reduced pressure, and then diluted by adding water (10.0 mL) and then the reaction mixture was extracted with EtOAc (2×25 mL), the combined extracts were dried over anhydrous Na₂SO₄, filtered, concentrated under reduced pressure to obtain the crude residue (220 mg) as a colorless viscous liquid. The crude material was purified by Combi-Flash column chromatography (100-200 silica gel) by eluting 0-40% EtOAc in hexanes to afford 31 (102.3 mg, 0.3033 mmol, 31% yield) as a solid. LCMS: 336.1 (M+H), Rₜ=2.192 min, Column: X-Bridge BEH C-18 (3.0×50 mm, 2.5 μm); Mobile Phase: A: 0.025% FA in Water, B: ACN; T/B %: 0.01/2, 0.2/2, 2/98, 3/98, 3.2/2, 4/2; Flow rate: 1.2 ml/min (Gradient); Column Oven temperature: 50° C. HPLC: Rₜ=10.164 min, 99.46%; Mobile Phase-A: 0.05% TFA:Acetonitrile (95:05); Mobile Phase-B: Acetonitrile: 0.05% TFA (95:05); Program: T/B %: 0.01/10, 12/90, 16/90; Flow: 1.0 mL/min; Diluent: ACN:Water. ¹H NMR (400 MHz, DMSO-d₆) δ 9.33 (t, 1H), 7.85 (d, 1H), 7.81 (d, 1H), 7.18 (d, 2H), 7.04 (d, 2H), 4.41 (d, 2H), 3.38 (s, 3H), 1.93-1.83 (m, 1H), 0.96-0.88 (m, 2H), 0.66-0.59 (m, 2H).

### Example 33. Synthesis of N-(4-(tert-butyl)benzyl)-5-(methylsulfonyl)thiophene-2-carboxamide (Compound 32)

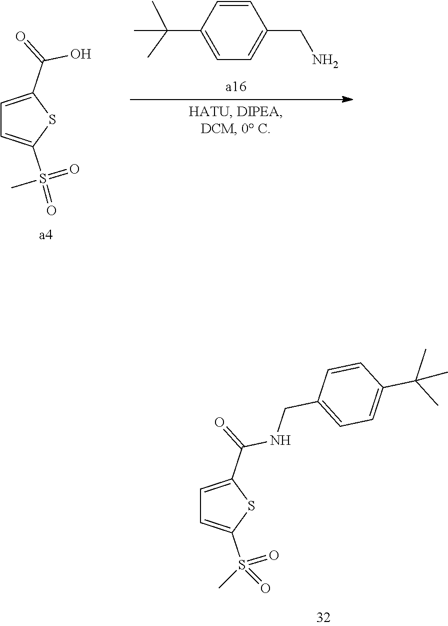 [SMILES: CS(=O)(=O)c1ccc(C(=O)O)s1 ; CC(C)(C)c1ccc(CN)cc1 ; CC(C)(C)c1ccc(CNC(=O)c2ccc(S(C)(=O)=O)s2)cc1]

To a stirred reaction mixture of 5-methylsulfonylthiophene-2-carboxylic acid (0.15 g, 0.7300 mmol) and a16 (0.18 g, 1.12 mmol) in DCM (5.00 mL) was added HATU (0.41 g, 1.09 mmol) followed by DIPEA (0.25 mL, 1.45 mmol) at 0° C., and stirring was continued further for 1 h at 0° C. The reaction mixture was concentrated under reduced pressure, and then diluted by adding water (10.0 mL) and then the reaction mixture was extracted with EtOAc (2×25 mL), the combined extracts were dried over anhydrous Na₂SO₄, filtered, concentrated under reduced pressure to obtain the crude residue (220 mg) as a colorless viscous liquid. The obtained crude was purified by Combi-Flash column chromatography (100-200 silica gel) by eluting 0-40% EtOAc in hexanes to afford 32 (114 mg, 0.32 mmol, 43% yield) as a solid. LCMS: 350.20 (M−H), Rₜ=2.004 min, Column: X-SELECT CSH C18 (50*3) mm 2.5u; Mobile Phase: A: 2.5 mMAmmonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. HPLC: Rₜ=10.56 min, 98.42%; MeMobile Phase A: 5 mM Ammonium Bi Carbonate; Mobile Phase B: Acetonitrile; Program: T/B %: 0.01/20, 12/90, 16/90; Flow: 1.0 mL/min; Diluent: WATER:ACN. ¹H NMR (400 MHz, DMSO-d6) δ 9.34 (t, 1H), 7.86 (d, 1H), 7.81 (d, 1H), 7.35 (d, 2H), 7.24 (d, 2H), 4.43 (d, 2H), 3.37 (s, 3H), 1.26 (s, 9H).

### Example 34. Synthesis of 5-(methylsulfonyl)-N-(4-(trifluoromethyl)benzyl)thiophene-2-carboxamide (Compound 33)

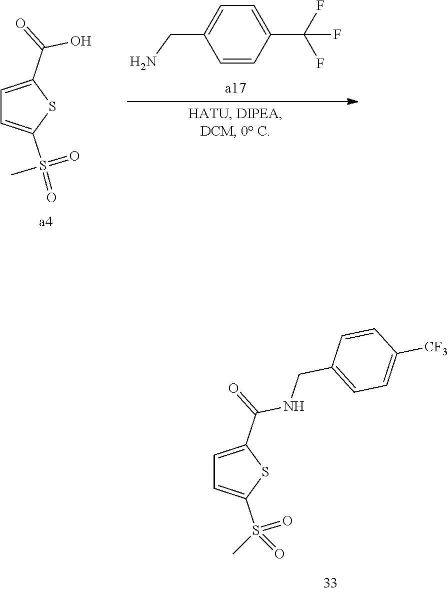 [SMILES: CS(=O)(=O)c1ccc(C(=O)O)s1 ; NCc1ccc(C(F)(F)F)cc1 ; CS(=O)(=O)c1ccc(C(=O)NCc2ccc(C(F)(F)F)cc2)s1]

To a stirred reaction mixture of 5-methylsulfonylthiophene-2-carboxylic acid (0.15 g, 0.7273 mmol) and a17 (0.18 g, 1.04 mmol) in DCM (5 mL) was added HATU (0.41 g, 1.09 mmol) followed by DIPEA (0.1880 mg, 1.45 mmol) at 0° C., then stirring was continued further for 1 h at 0° C. The reaction mixture was concentrated under reduced pressure, and then diluted by adding water (10.0 mL) and then the reaction mixture was extracted with EtOAc (2×25 mL), the combined extracts were dried over anhydrous Na₂SO₄, filtered, concentrated under reduced pressure to obtain the crude residue (250 mg) as a viscous liquid. The obtained crude was purified by Combi-Flash column chromatography (100-200 silica gel) by eluting 0-40% EtOAc in hexanes to afford 33 (121 mg, 0.33 mmol, 45% yield) as a solid. LCMS: 362.10 (M−H), Rₜ=2.206 min, Column: X-SELECT CSH C18 (50*3) mm 2.5u; Mobile Phase: A: 2.5 mMAmmonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. HPLC: Rₜ=10.211 min, 98.63%; Column: XSELECT CSH C18 (150×4.6 mm, 3.5); Mobile Phase-A: 0.05% TFA: Acetonitrile (95:05); Mobile Phase-B: Acetonitrile: 0.05% TFA (95:05); Program: T/B %: 0.01/10, 12/90, 16/90; Flow: 1.0 mL/min; Diluent: ACN:Water. ¹H NMR (400 MHz, DMSO-d6) δ 9.50 (t, 1H), 7.88 (d, 1H), 7.83 (d, 1H), 7.71 (d, 2H), 7.54 (d, 2H), 4.57 (d, 2H), 3.39 (s, 3H).

### Example 35. Synthesis of 5-(methylsulfonyl)-N-(4-(2,2,2-trifluoroethoxy)benzyl)thiophene-2-carboxamide (Compound 34)

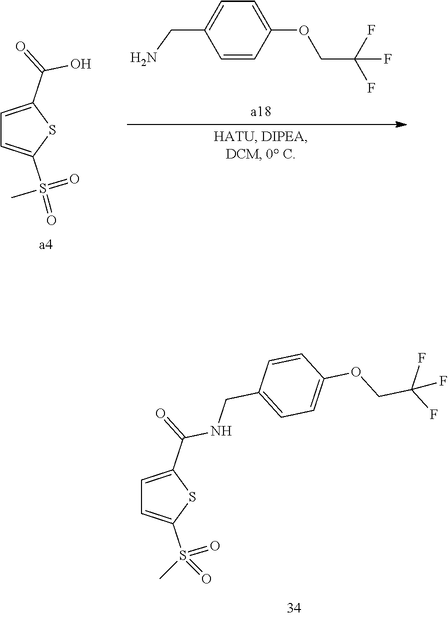 [SMILES: CS(=O)(=O)c1ccc(C(=O)O)s1 ; NCc1ccc(OCC(F)(F)F)cc1 ; CS(=O)(=O)c1ccc(C(=O)NCc2ccc(OCC(F)(F)F)cc2)s1]

To a stirred reaction mixture of 5-methylsulfonylthiophene-2-carboxylic acid (0.15 g, 0.7273 mmol) and a18 (0.1829 g, 0.8914 mmol) in DCM (20 mL) was added HATU (0.41 g, 1.09 mmol) followed by DIPEA (0.1880 mg, 1.45 mmol) at 0° C., then stirring was continued further for 1 h at 0° C. The reaction mixture was concentrated under reduced pressure, diluted by adding water (10.0 mL) and then the reaction mixture was extracted with EtOAc (2×25 mL). The combined extracts were dried over anhydrous Na₂SO₄, filtered, concentrated under reduced pressure to obtain the crude residue (270 mg) as a viscous liquid. The obtained crude was purified by Combi-Flash column chromatography (100-200 silica gel) by eluting 0-40% EtOAc in hexanes to afford 34 (211 mg, 0.52 mmol, 72% yield) as a solid. LCMS: 392.15 (M−H), Rₜ=1.891 min, Column: X-SELECT CSH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. HPLC: Rₜ=8.084 min, 97.87%; Column: XSELECT CSH C18 (150×4.6 mm, 3.5i); Mobile Phase-A: 0.1% Formic acid in Water; Mobile Phase-B: Acetonitrile; Program: T/B %: 0.01/5, 1.0/5, 8.0/100, 12.0/100, 14.0/5, 18.0/5; Flow: 1.0 mL/min; Diluent: ACN:Water. ¹H NMR (400 MHz, DMSO-d6) δ 9.35 (t, 1H), 7.85 (d, 1H), 7.81 (d, 1H), 7.28 (d, 2H), 7.06-7.00 (m, 2H), 4.73 (q, H), 4.42 (d, 2H), 3.38 (s, 3H).

### Example 36. Synthesis of 5-(N-methylsulfamoyl)-N-(4-(2,2,2-trifluoroethoxy)benzyl)thiophene-2-carboxamide (Compound 35)

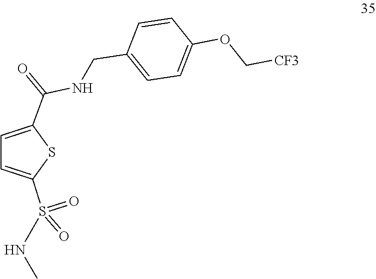 [SMILES: CNCS(=O)(=O)c1ccc(C(=O)NCc2ccc(OCC(F)(F)F)cc2)s1]

To a stirred solution of a3 (200 mg, 0.9040 mmol) in DMF (10 mL) was added DIPEA (323 mg, 2.499 mmol), HATU (443 mg, 1.1651 mmol) followed by corresponding amine (244 mg, 1.1892 mmol) at 0° C. The reaction mixture was stirred at room temperature for 16 h. The reaction mass was quenched with water, extracted with EtOAc (50 mL×2). The combined organic layer was washed with water, brine, dried over anhydrous Na₂SO₄ and concentrated under reduced pressure. The crude product was purified by flash column chromatography using EtOAc in heptane: 0% to 35% to 90% as eluent to afford the titled compound 35 (95 mg, 0.23 mmol, 25%). LCMS: 409.00 (M+H), Rₜ=2.066 min, Column: X-SELECT CSH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. HPLC: Rₜ=9.51 min, 97.73%; Column: X SELECT CSH C18(150×4.6 mm, 3.5u); Mobile Phase A: 5 mM AMMONIUM BICARABONATE; Mobile Phase B: ACETONITRILE; Program: T/B %: 0.01/2, 2/2, 12/90, 16/90; Flow: 1.0 mL/min; Diluent: ACN:WATER. ¹H NMR (400 MHz, DMSO-d6) δ 9.30 (t, 1H), 7.80 (d, 2H), 7.57 (d, 1H), 7.28 (d, 2H), 7.03 (d, 2H), 4.79-4.67 (m, 2H), 4.41 (d, 2H), 3H merged in solvent peak.

### Example 37. Synthesis of 5-(N-methylsulfamoyl)-N-(4-(1-(trifluoromethyl)cyclopropyl)benzyl)thiophene-2-carboxamide (Compound 36)

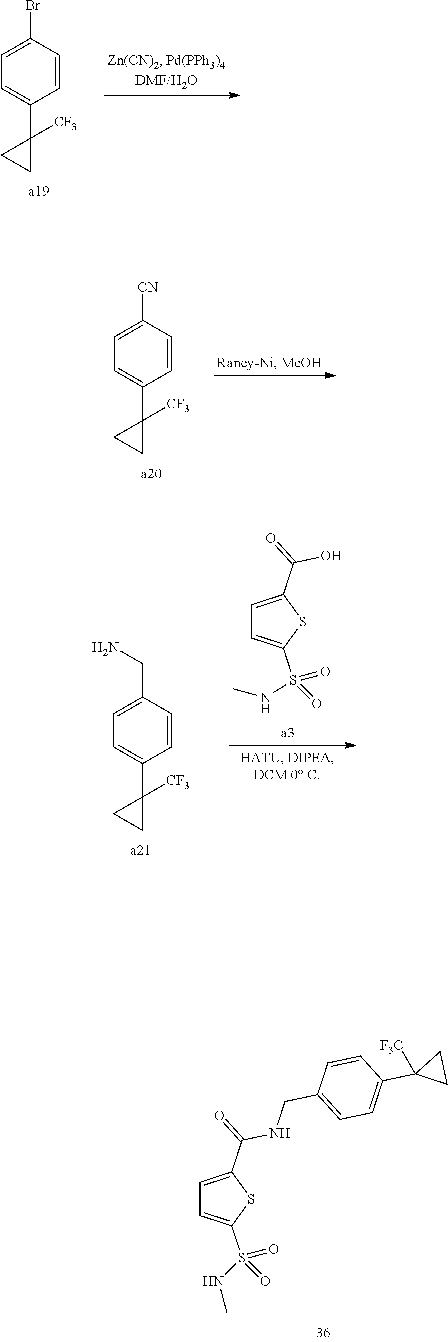 [SMILES: FC(F)(F)C1(c2ccc(Br)cc2)CC1 ; N#Cc1ccc(C2(C(F)(F)F)CC2)cc1 ; NCc1ccc(C2(C(F)(F)F)CC2)cc1 ; CNS(=O)(=O)c1ccc(C(=O)O)s1 ; CNS(=O)(=O)c1ccc(C(=O)NCc2ccc(C3(C(F)(F)F)CC3)cc2)s1]

### Synthesis of 4-(1-(trifluoromethyl)cyclopropyl)benzonitrile (a20)

To a stirred solution of a19 (3 g, 11.32 mmol) in DMF (15 mL) was added zinc cyanide (0.9256 g, 7.9111 mmol) and Pd(PPh₃)₄ (0.78 g, 0.6800 mmol) under argon atmosphere. The reaction mixture was then stirred at 80° C. overnight. The reaction mixture was allowed to cool to room temperature followed by addition of ZnCN₂ (0.93 g, 7.91 mmol) and Pd(PPh₃)₄ (0.78 g, 0.6800 mmol). The reaction mixture was stirred at 120° C. for 5 h. The reaction mixture was allowed to cool to room temperature, filtered and washed filtered cake with DMF. The filtrate was concentrated under reduced pressure, added ethyl acetate, washed twice with 2 M aqueous ammonia solution followed by saturated aqueous sodium chloride solution, dried over anhydrous Na₂SO₄ and concentrated under reduced pressure. The obtained crude was purified by flash column chromatography to afford a20 (1 g, 4.69 mmol, 41% yield).

### Synthesis of (4-(1-(trifluoromethyl)cyclopropyl)phenyl)methanamine (a21)

To a stirred solution of a20 (800 mg, 3.79 mmol) in MeOH (16 mL) was added Raney Nickel (659 mg, 11.36 mmol) and hydrogenated (100 psi) at room temperature for 3 h. The reaction mixture was filtered through a pad of celite. The filtrate was concentrated under reduced pressure. The crude compound was purified by prep HPLC to obtain afford a21 (244 mg, 1.13 mmol, 29% yield).

### Synthesis of 5-(N-methylsulfamoyl)-N-(4-(1-(trifluoromethyl)cyclopropyl)benzyl)-thiophene-2-carboxamide (Compound 36)

To a stirred solution of a3 (160 mg, 0.72 mmol) in DCM (15 mL) was added DIPEA (0.44 mL, 2.5 mmol), HATU (443 mg, 1.17 mmol) at 0° C. and stirred for 10 minutes followed by the addition of a21 (244 mg, 1.13 mmol) at the same temperature. The reaction mixture was allowed to warm to room temperature and stirred for 1 h. The reaction mixture was quenched with water (10 mL) and extracted with DCM (2×30 mL). The combined organic layers were washed with water (10 mL), brine (10 mL), dried over anhydrous Na₂SO₄ and concentrated under reduced pressure. The obtained crude was purified by flash chromatography on silica gel using EtOAc in heptane 0% to 90% as eluent to afford 36 (67.4 mg, 0.1567 mmol, 22% yield). HPLC: Rt 10.491 min, 97.29%; Mobile Phase A: 5 mM Ammonium Bicarbonate; Mobile Phase B: Acetonitrile; Program: T/B %: 0.01/20, 12/90, 16/90; Flow: 1.0 mL/min; Diluent: ACN:WATER. LCMS: 417.15 (M−H), Rt 2.088 min, Column: X-SELECT CSH C18 (50*3) mm 2.5u; Mobile Phase: A: 2.5 mM Ammonium Bicarbonate in water; B: Acetonitrile; Inj Volume: 2 L, Flow Rate: 1.2 mL/minute; Column oven temp. 50° C.; Gradient program: 0% B to 98% B in 2.0 minute, hold till 3.0 min, at 3.2 min B conc is 0% up to 4.0 min. ¹H NMR (400 MHz, DMSO-d6) δ 9.33 (t, 1H), 7.85-7.78 (m, 2H), 7.58 (d, 1H), 7.43 (d, 2H), 7.33 (d, 2H), 4.46 (d, 2H), 2.58-2.52 (m, 3H), 1.38-1.27 (m, 2H), 1.09 (br s, 2H).

### Example 38. Synthesis of 5-(methylsulfonyl)-N-(4-(1-(trifluoromethyl)cyclopropyl)benzyl)thiophene-2-carboxamide (Compound 37)

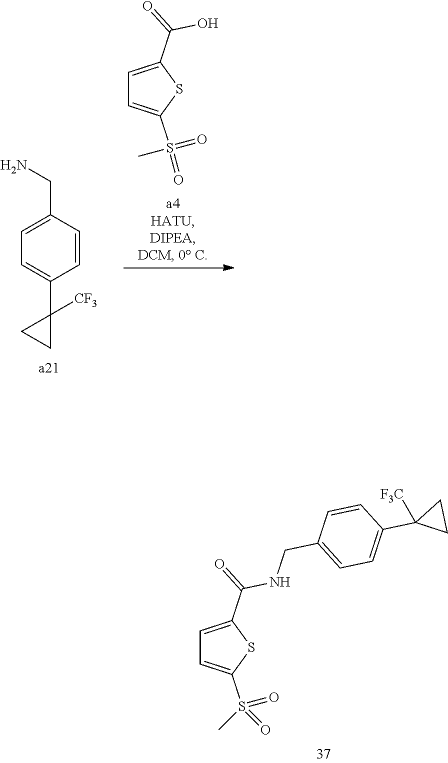 [SMILES: NCc1ccc(C2(C(F)(F)F)CC2)cc1 ; CS(=O)(=O)c1ccc(C(=O)O)s1 ; CS(=O)(=O)c1ccc(C(=O)NCc2ccc(C3(C(F)(F)F)CC3)cc2)s1]

To a stirred solution of a4 (50 mg, 0.2400 mmol) in DCM (5 mL) was added DIPEA (0.08 mL, 0.48 mmol), HATU (118 mg, 0.31 mmol) at 0° C. and stirred for 10 minutes followed by the addition of a21 (52 mg, 0.24 mmol) at the same temperature. The reaction mixture was allowed to warm to room temperature and stirred for 1 h. The reaction mixture was quenched with water (10 mL) and extracted with DCM (2×30 mL). The combined organic layers were washed with water (10 mL), brine (10 mL), dried over anhydrous Na₂SO₄ and concentrated under reduced pressure. The obtained crude product was then purified by flash chromatography on silica gel using EtOAc in heptane 0% to 90% as eluent to afford 37 (27.31 mg, 0.0647 mmol, 28% yield). HPLC: Rt 10.143 min, 95.60%; Mobile Phase A: 5 mM Ammonium Bicarbonate; Mobile Phase B: Acetonitrile; Program: T/B %: 0.01/20, 12/90, 16/90; Flow: 1.0 mL/min; Diluent: ACN:WATER. LCMS: 402.30 (M−H), Rt 2.106 min, Column: X-SELECT CSH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. ¹H NMR (400 MHz, DMSO-d6) δ 9.41 (t, 1H), 7.88-7.80 (m, 2H), 7.43 (d, 2H), 7.27 (d, 2H), 4.46 (d, 2H), 3.38 (s, 3H), 1.36-1.28 (m, 2H), 1.08 (br s, 2H).

### Example 39. Synthesis of N-(4-(2-cyanopropan-2-yl)benzyl)-5-(N-methylsulfamoyl)thiophene-2-carbox-amide (Compound 38)

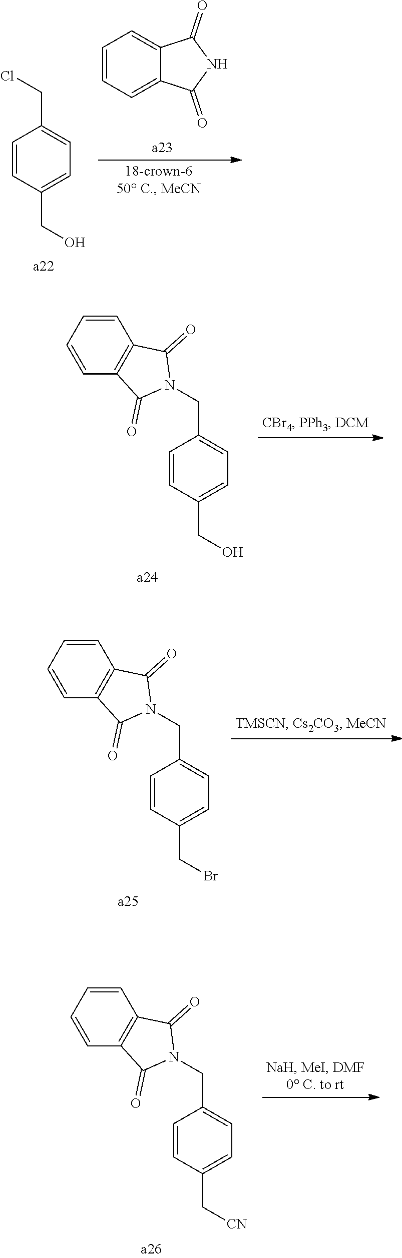 [SMILES: OCc1ccc(CCl)cc1 ; O=C1NC(=O)c2ccccc21 ; O=C1c2ccccc2C(=O)N1Cc1ccc(CO)cc1 ; O=C1c2ccccc2C(=O)N1Cc1ccc(CBr)cc1 ; N#CCc1ccc(CN2C(=O)c3ccccc3C2=O)cc1]

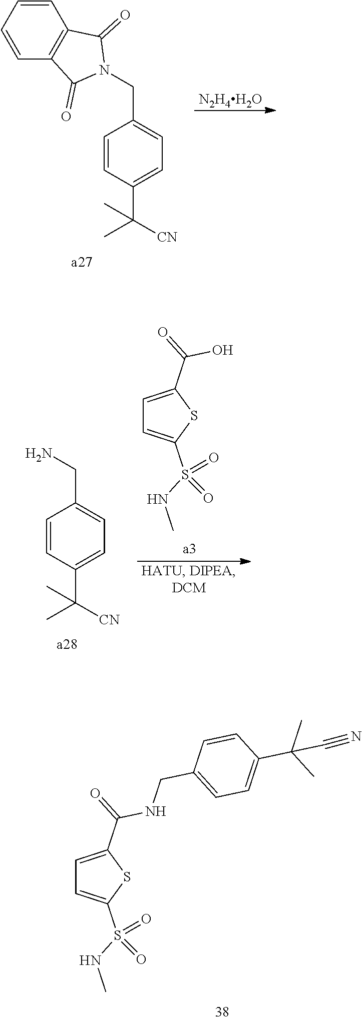 [SMILES: CC(C)(C#N)c1ccc(CN2C(=O)c3ccccc3C2=O)cc1 ; "N2H4·H2O" ; CC(C)(C#N)c1ccc(CN)cc1 ; CNS(=O)(=O)c1ccc(C(=O)O)s1 ; "HATU, DIPEA,DCM" ; CNS(=O)(=O)c1ccc(C(=O)NCc2ccc(C(C)(C)C#N)cc2)s1]

### Synthesis of 2-(4-(hydroxymethyl)benzyl)isoindoline-1,3-dione (a24)

To a stirred solution of a22 (1 g, 6.39 mmol) in MeCN (80 mL) was added a23 (1.32 g, 8.94 mmol) and 18-crown-6 (0.24 g, 0.89 mmol). The reaction mixture was stirred at 50° C. for 16 h. The reaction mixture was filtered, washed the filtered cake with ethyl acetate (50 mL), then the filtrate organic layer was concentrated under reduced pressure. The obtained crude product was purified by column chromatography (100-200 silica) using 20-30% ethyl acetate in hexane as an eluent to afford a24 (1 g, 3.704 mmol, 58% yield) as a solid.

### Synthesis of 2-(4-(bromomethyl)benzyl)isoindoline-1,3-dione (a25)

To a stirred solution of a24 (1 g, 3.74 mmol) in DCM (20 mL) was added PPh₃ (1.963 g, 7.48 mmol) and CBr₄ (1.58 mL, 7.48 mmol) at 0° C. then the reaction mixture was stirred at room temperature for 16 h. The reaction mixture was quenched using water (20 mL) and extracted with DCM (2×20 mL). The combined organic layers were dried over anhydrous Na₂SO₄, concentrated under reduced pressure. The obtained crude was purified by column chromatography (100-200 silica) using 20-30% ethyl acetate in hexane as an eluent to afford a25 (600 mg, 1.7627 mmol, 47% yield) as a solid.

### Synthesis of 2-(4-((1,3-dioxoisoindolin-2-yl)methyl)phenyl)acetonitrile (a26)

To a stirred solution of a25 (500 mg, 1.51 mmol) in MeCN (10 mL) was added TMSCN (0.21 mL, 1.67 mmol) and Cs₂CO₃ (986.8 mg, 3.03 mmol) at room temperature and then stirring was continued for 3 h at 80° C. The reaction mixture was diluted with water (10 mL) and extracted with ethyl acetate (2×20 mL). The combined organic layers were dried over anhydrous Na₂SO₄, concentrated under reduced pressure. The crude material was purified by column chromatography (100-200 silica) using 20-30% ethyl acetate in hexane as an eluent to afford the a26 (350 mg, 1.2034 mmol, 79% yield) as a solid.

### Synthesis of 2-(4-((1,3-dioxoisoindolin-2-yl)methyl)phenyl)-2-methylpropanenitrile (a27)

To a stirred solution of a26 (200 mg, 0.72 mmol) in DMF (2 mL) was added NaH (34.75 mg, 1.45 mmol) at 0° C., stirred for 10 minutes followed by the addition of iodomethane (0.09 mL, 1.45 mmol). The reaction mixture was then stirred at room temperature for 18 h. The reaction mixture was diluted with water (10 mL) and extracted with ethyl acetate (2×20 mL), combined organic layers were dried over anhydrous Na₂SO₄ and concentrated under reduced pressure. The obtained crude was purified by column chromatography (100-200 silica) using 30-40% ethyl acetate in hexane as an eluent to afford a27 (70 mg, 0.1656 mmol, 23% yield) as a solid.

### Synthesis of 2-(4-(aminomethyl)phenyl)-2-methylpropanenitrile (a28)

To a stirred solution of a27 (70 mg, 0.23 mmol) in ethanol (0.2 mL)/DCM (1 mL) was added N₂H₄·H₂O (12.65 mg, 0.25 mmol) and then stirring was continued at room temperature for overnight. The reaction mixture was concentrated under reduced pressure. The obtained crude was diluted with aqueous NaOH (2 mL) and extracted with diethyl ether (5 mL), dried over anhydrous Na₂SO₄ and concentrated under reduced pressure to give a28 (20 mg, 0.07 mmol, 30% yield) as a liquid.

### Synthesis of N-(4-(2-cyanopropan-2-yl)benzyl)-5-(N-methylsulfamoyl)thiophene-2-carboxamide (Compound 38)

To a stirred solution of a3 (190.47 mg, 0.86 mmol) in DCM (2 mL) were added DIPEA (0.2 mL, 1.15 mmol), HATU (327 mg, 0.86 mmol) at 0° C. and stirred for 10 minutes followed by the addition of a28 (100.mg, 0.57 mmol) at the same temperature. The reaction mixture was stirred at room temperature for 2 h. The reaction mixture was quenched with water (10 mL) and extracted with DCM (2×5 mL). The combined organic layers were washed with water (10 mL), brine (10 mL), dried over anhydrous Na₂SO₄ and concentrated under reduced pressure. The obtained crude product was purified by flash chromatography (100-200 silica) using 30-50% ethyl acetate in hexane as an eluent followed by prep-HPLC to afford 38 (70 mg, 0.183 mmol, 32% yield). HPLC: Rt 7.693 min, 98.70%; Column: XSELECT CSH C18 (150×4.6 mm, 3.5); Mobile Phase-A: 0.1% FA in Water; Mobile Phase-B: Acetonitrile; Program: T/B %: 0.01/5, 1/5, 8/100, 12/100, 14/5, 18/5; Flow: 1.2 mL/min. LCMS: 378.90 (M+H), Rt 1.874 min, Column: X-SELECT CSH C18 (50*3) mm 2.5u; Mobile Phase: A: 2.5 mM Ammonium Bicarbonate in water; B: Acetonitrile; Inj Volume: 2 L, Flow Rate: 1.2 mL/minute; Column oven temp. 45° C.; Gradient program: 0% B to 98% B in 2.0 minute, hold till 3.0 min, at 3.2 min B conc is 0% up to 4.0 min. ¹H NMR (400 MHz, CHLOROFORM-d) δ 7.53 (d, 1H), 7.50-7.45 (m, 2H), 7.41 (d, 1H), 7.40-7.36 (m, 2H), 6.31 (br s, 1H), 4.63 (d, 2H), 4.50-4.43 (m, 1H), 2.78 (d, 3H), 1.72 (s, 6H).

### Example 40. Synthesis of N-(4-(2-cyanopropan-2-yl)benzyl)-5-(methylsulfonyl)thiophene-2-carboxamide (Compound 39)

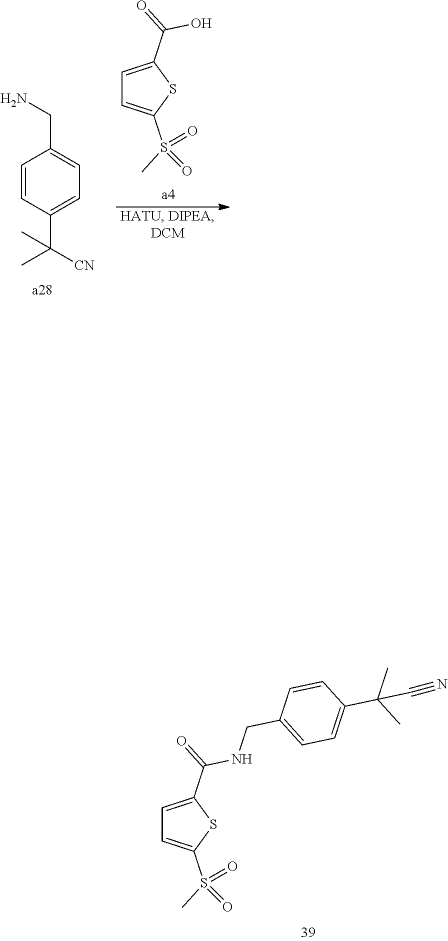 [SMILES: CC(C)(C#N)c1ccc(CN)cc1 ; CS(=O)(=O)c1ccc(C(=O)O)s1 ; "HATU, DIPEA,DCM" ; CC(C)(C#N)c1ccc(CNC(=O)c2ccc(S(C)(=O)=O)s2)cc1]

To a stirred solution of a4 (190.47 mg, 0.92 mmol) in DCM (2 mL) were added DIPEA (0.2 mL, 1.15 mmol), HATU (327 mg, 0.86 mmol) at 0° C. and stirred for 10 minutes followed by the addition of a28 (100 mg, 0.5700 mmol) at the same temperature. The reaction mixture was stirred at room temperature for 2 h. The reaction mixture was quenched with water (10 mL) and extracted with DCM (2×5 mL). The combined organic layer was washed with water (10 mL), brine (10 mL), dried over anhydrous Na₂SO₄ and concentrated under reduced pressure. The obtained crude product was purified by flash chromatography (100-200 silica) using 30-50% ethyl acetate in hexane as an eluent followed by prep-HPLC to afford 39 (60 mg, 0.1654 mmol, 29% yield). HPLC: Rt 7.660 min, 99.94%; Column: XSELECT CSH C18 (150×4.6 mm, 3.5); Mobile Phase-A: 0.1% FA in Water; Mobile Phase-B: Acetonitrile; Program: T/B %: 0.01/5, 1/5, 8/100, 12/100, 14/5, 18/5; Flow: 1.2 mL/min. LCMS: 363.1 (M+H), Rt 1.928 min, Column: X-Bridge BEH C-18 (3.0×50 mm, 2.5 μm); Mobile Phase: A: 0.025% FA in Water, B: ACN; T/B %: 0.01/2, 0.2/2, 2.2/98, 3/98, 3.2/2, 4/2; Flow rate: 1.2 ml/min(Gradient); Column Oven temperature: 50° C. ¹H NMR (400 MHz, CHLOROFORM-d) δ 7.65 (d, 1H), 7.52-7.42 (m, 3H), 7.41-7.34 (m, 2H), 6.35 (br s, 1H), 4.63 (d, 2H), 3.20 (s, 3H), 1.72 (s, 6H).

### Example 41. Synthesis of N-(4-(isopropylamino)benzyl)-5-(N-methylsulfamoyl)thiophene-2-carboxamide (Compound 40)

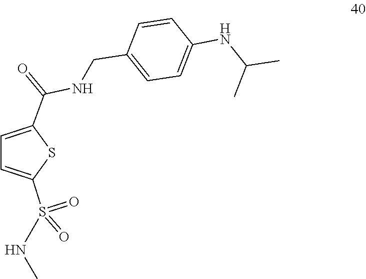 [SMILES: CNCS(=O)(=O)c1ccc(C(=O)NCc2ccc(NC(C)C)cc2)s1]

To a stirred solution of a3 (160 mg, 0.7200 mmol) in DCM (15 mL) was added DIPEA (0.44 mL, 2.5 mmol), HATU (443 mg, 1.17 mmol) at 0° C. and stirred for 10 minutes followed by the addition of the corresponding amine (244 mg, 1.49 mmol) at the same temperature. The reaction mixture was stirred at room temperature for 1 h. The reaction mixture was quenched with water (15 mL) and extracted with DCM (2×30 mL). The combined organic layers were washed with water (10 mL), brine (10 mL), dried over anhydrous Na₂SO₄ and concentrated under reduced pressure. The obtained crude product was purified by flash chromatography on silica gel using 0 to 90% EtOAc in heptane as eluent followed by prep-HPLC to afford 40 (35 mg, 0.093 mmol, 13% yield). HPLC: Rt 8.43 min, 97.47%; Column: X SELECT CSH C18 (150×4.6 mm, 3.5μ; Mobile Phase A: 5 mM Ammonium Bi Carbonate; Mobile Phase B: Acetonitrile; Program: T/B %: 0.01/10, 12/90, 16/90; Flow: 1.0 mL/min; Diluent: WATER:ACN:DMSO. LCMS: 368.1 (M+H), Rt 1.505 min, Column: X-Bridge BEH C-18 (3.0×50 mm, 2.5 μm); Mobile Phase: A: 0.025% FA in Water, B: ACN; T/B %: 0.01/2, 0.2/2, 2.2/98, 3/98, 3.2/2, 4/2; Flow rate: 1.2 ml/min (Gradient); Column Oven temperature: 50° C. ¹H NMR (400 MHz, DMSO-d6) δ 9.13 (br t, 1H), 7.82-7.76 (m, 2H), 7.56 (d, 1H), 7.02 (d, 2H), 6.51 (d, 2H), 5.26 (d, 1H), 4.28 (d, 2H), 3.56-3.45 (m, 1H), 2.52 (br s, 3H), 1.10 (d, 6H).

### Example 42. Synthesis of N-(4-(isopropylamino)benzyl)-5-(methylsulfonyl)thiophene-2-carboxamide (Compound 41)

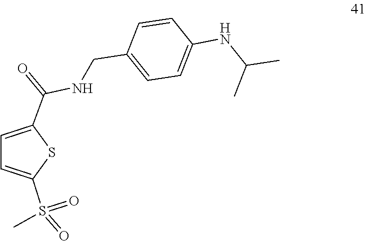 [SMILES: CC(C)Nc1ccc(CNC(=O)c2ccc(S(C)(=O)=O)s2)cc1]

To a stirred solution of a4 (160 mg, 0.7800 mmol) in DCM (15 mL) was added DIPEA (0.44 mL, 2.5 mmol), HATU (443 mg, 1.17 mmol) at 0° C. and stirred for 10 minutes followed by the addition of corresponding amine (244 mg, 1.49 mmol) at the same temperature. The reaction mixture was stirred at room temperature for 1 h. The reaction mixture was quenched with water (10 mL) and extracted with DCM (2×30 mL). The combined organic layer was washed with water (10 mL), brine (10 mL), dried over anhydrous Na₂SO₄ and concentrated under reduced pressure. The obtained crude product was purified by flash chromatography on silica gel using 0% to 90% EtOAc in hexane as eluent followed by prep-HPLC to afford 41 (60 mg, 0.16 mmol, 21% yield). HPLC: Rt 8.451 min, 96.42%; Column: X SELECT CSH C18 (150×4.6 mm, 3.5); Mobile Phase A: 5 mM Ammonium Bicarbonate; Mobile Phase B: Acetonitrile; Program: T/B %: 0.01/20, 12/90, 16/90; Flow: 1 mL/min; Diluent: WATER:ACN. LCMS: 353.0 (M+H), Rt 1.747 min, Column: X-SELECT CSH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 45° C.; Gradient program: 0% B to 98% B in 2.0 minute, hold till 3.0 min, at 3.2 min B conc is 0% up to 4.0 min. ¹H NMR (400 MHz, DMSO-d6) δ 9.20 (br t, 1H), 7.85 (d, 1H), 7.79 (d, 1H), 7.02 (d, 2H), 6.51 (d, 2H), 5.27 (d, 1H), 4.29 (d, 2H), 3.56-3.45 (m, 1H), 3.37 (s, 3H), 1.10 (d, 6H).

### Example 43. Synthesis of N-(2-fluoro-4-(trifluoromethyl)benzyl)-5-(methylsulfonyl)thiophene-2-carboxamide (Compound 42)

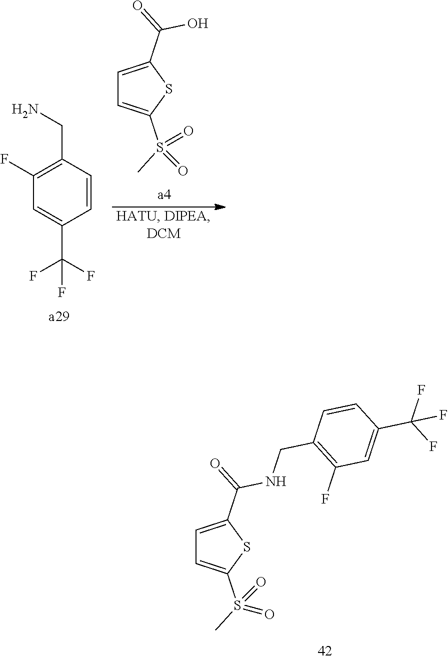 [SMILES: NCc1ccc(C(F)(F)F)cc1F ; CS(=O)(=O)c1ccc(C(=O)O)s1 ; "HATU, DIPEA,DCM" ; CS(=O)(=O)c1ccc(C(=O)NCc2ccc(C(F)(F)F)cc2F)s1]

To a stirred solution of a4 (300 mg, 1.45 mmol) and a29 (337.13 mg, 1.75 mmol) in DCM (10 mL) was added DIPEA (0.38 mL, 2.18 mmol) and HATU (829.63 mg, 2.18 mmol) at 0° C. and the reaction was stirred at 0° C. for 1 h. The reaction mixture was concentrated to dryness and the residue was diluted with EtOAc (30 mL) and washed (10 mL) with water followed by saturated brine solution (10 mL), dried over MgSO₄ and concentrated under reduced pressure. The crude was then purified by flash column chromatography eluting with 60% EtOAc/Heptane to afford 42 (208 mg, 0.54 mmol, 37% yield). HPLC: Rt 8.270 min, 99.99%; Column: XSELECT CSH C18 (150×4.6 mm, 3.5); Mobile Phase-A: 0.1% FA in Water; Mobile Phase-B: Acetonitrile; Program: T/B %: 0.01/5, 1/5, 8/100, 12/100, 14/5, 18/5; Flow: 1.2 mL/min. ¹H NMR (400 MHz, DMSO-d6) δ 9.47 (t, 1H), 7.89 (d, 1H), 7.83 (d, 1H), 7.69 (d, 1H), 7.64-7.55 (m, 2H), 4.58 (d, 2H), 3.38 (s, 3H).

### Example 44. Synthesis of N-(2-fluoro-4-(trifluoromethyl)benzyl)-5-(N-methylsulfamoyl)thiophene-2-carboxamide (Compound 43)

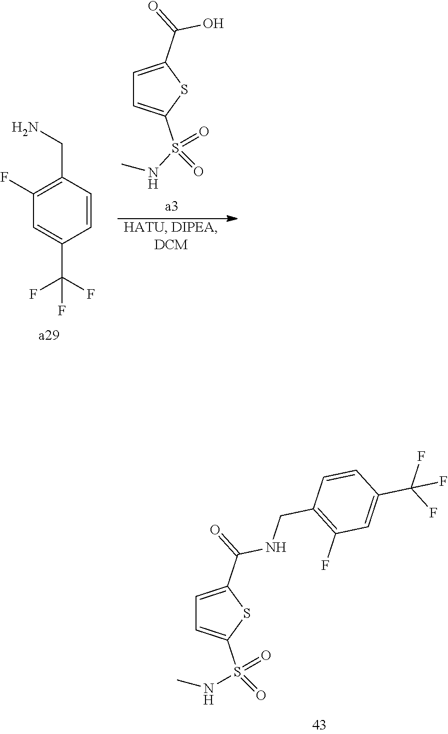

To a stirred solution of a3 (300 mg, 1.36 mmol) and a29 (314.26 mg, 1.63 mmol) in DCM (10 mL) was added DIPEA (0.35 mL, 2.03 mmol) and HATU (773.35 mg, 2.03 mmol) at 0° C. and the reaction mixture was stirred at 0° C. for 1 h. The reaction mixture was concentrated to dryness and the residue was diluted with EtOAc (30 mL) and washed with water (10 mL) followed by saturated brine solution (10 mL), dried over MgSO₄ and concentrated under reduced pressure. The crude was then purified by flash column chromatography eluting with 60% EtOAc/Heptane to afford 43 (139.34 mg, 0.3503 mmol, 25.8% yield). HPLC: Rt 8.266 min, 99.67%; Column: XSELECT CSH C18 (150×4.6 mm, 3.5); Mobile Phase-A: 0.1% FA in Water; Mobile Phase-B: Acetonitrile; Program: T/B %: 0.01/5, 1/5, 8/100, 12/100, 14/5, 18/5; Flow: 1.2 mL/min. ¹H NMR (400 MHz, DMSO-d₆) δ 9.41 (t, 1H), 7.86-7.79 (m, 2H), 7.68 (d, 1H), 7.64-7.56 (m, 3H), 4.57 (d, 2H), 2.52 (br d, 3H).

### Example 45. Synthesis of N-(2-methyl-4-(trifluoromethyl)benzyl)-5-(methylsulfonyl)thiophene-2-carboxamide (Compound 44)

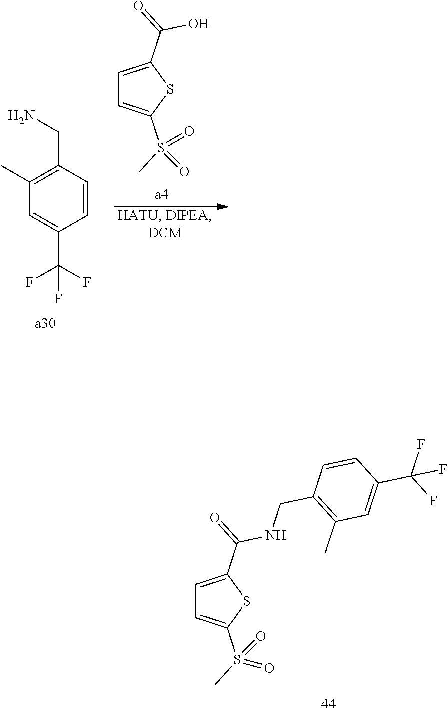 [SMILES: Cc1cc(C(F)(F)F)ccc1CN ; CS(=O)(=O)c1ccc(C(=O)O)s1 ; "HATU, DIPEA,DCM" ; Cc1cc(C(F)(F)F)ccc1CNC(=O)c1ccc(S(C)(=O)=O)s1]

To a stirred solution of a4 (245.29 mg, 1.19 mmol) and a30 (150.mg, 0.79 mmol) in DCM (5 mL) was added DIPEA (0.41 mL, 2.38 mmol) and HATU (452.22 mg, 1.19 mmol) at and the reaction mass was stirred at room temperature for 2 h. The reaction mixture was quenched with water (10 mL) and extracted with DCM (2×25 mL). The combined organic layers were washed with water (50 mL), brine (20 mL), dried over anhydrous Na₂SO₄ and concentrated under reduced pressure. The obtained crude product was purified by flash chromatography on silica gel using 30-50% EtOAc in hexane as an eluent to afford 44 (130 mg, 0.33 mmol, 41% yield). HPLC: Rt 7.799 min, 95.115%; Column: X Select CSH C18(150×4.6)mm, 3.5μ; Mobile phase A: 0.1% FA in Water:ACN (95:05); Mobile phase B: Acetonitrile; Gradient Program: T/B %: 0.01/5, 1/5, 8/100, 12/100, 14/5, 18/5; Flow rate: 1.2 ml/min. LCMS: 378.0 (M+H), Rt 1.806 min, Column: X-Select CSH (3.0*50) mm 2.5u; Mobile Phase: A: 0.05% Formic acid in water:ACN (95:5); B: 0.05% Formic acid in ACN; Inj Volume: 2.0 μL; Flow Rate: 1.2. mL/minute; Column oven temperature: 50° C.; Gradient program: 0% B to 98% B in 2.0 min, hold till 3.0 min, at 3.2 min B conc is 0% up to 4.0 min. ¹H NMR (400 MHz, DMSO-d₆) δ 9.37 (t, 1H), 7.86-7.79 (m, 2H), 7.59-7.41 (m, 3H), 4.57 (d, 2H), 3.38 (s, 3H), 2.40 (s, 3H).

### Example 46. Synthesis of N-(2-methyl-4-(trifluoromethyl)benzyl)-5-(N-methylsulfamoyl)thiophene-2-carboxamide (Compound 45)

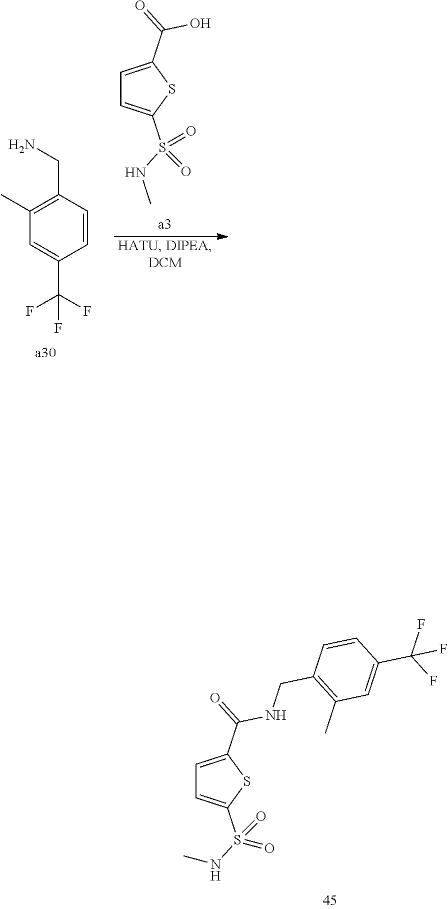 [SMILES: Cc1cc(C(F)(F)F)ccc1CN ; CNS(=O)(=O)c1ccc(C(=O)O)s1 ; "HATU, DIPEA,DCM" ; CNS(=O)(=O)c1ccc(C(=O)NCc2ccc(C(F)(F)F)cc2C)s1]

To a stirred solution of a30 (150 mg, 0.7900 mmol) and a3 (263.14 mg, 1.19 mmol) in DCM (5 mL) was added HATU (452.22 mg, 1.19 mmol) followed by DIPEA (0.41 mL, 2.38 mmol) at room temperature. The reaction mixture was stirred at room temperature for 2 h. The reaction mixture was quenched with water (10 mL) and extracted with DCM (2×25 mL). The combined organic layers were washed with water (50 mL), brine (20 mL), dried over anhydrous Na₂SO₄ and concentrated under reduced pressure. The obtained crude product was purified by flash chromatography on silica gel using 50-90% EtOAc in hexane as an eluent to afford 45 (90 mg, 0.22 mmol, 28% yield). HPLC: Rt 7.804 min, 96.260%; Column: X Select CSH C18(150×4.6)mm, 3.5μ; Mobile phase A: 0.1% FA in Water:ACN (95:05); Mobile phase B: Acetonitrile; Gradient Program: T/B %: 0.01/5, 1/5, 8/100, 12/100, 14/5, 18/5; Flow rate: 1.2 ml/min. LCMS: 393.1 (M+H), Rt 2.559 min, Column: X-Bridge BEH C-18 (3.0×50 mm, 2.5 μm); Mobile Phase: A: 0.025% FA in Water, B: ACN; T/B %: 0.01/2, 0.2/2, 2.2/98, 3/98, 3.2/2, 4/2; Flow rate: 1.2 ml/min(Gradient); Column Oven temperature: 50° C. ¹H NMR (400 MHz, METHANOL-d4) δ 7.72 (d, 1H), 7.56 (d, 1H), 7.51-7.43 (m, 3H), 4.62 (s, 2H), 2.64 (s, 3H), 2.45 (s, 3H).

### Example 47. Synthesis of 5-(methylsulfonyl)-N-((6-(trifluoromethyl)pyridin-3-yl)methyl)thiophene-2-carboxamide (Compound 46)

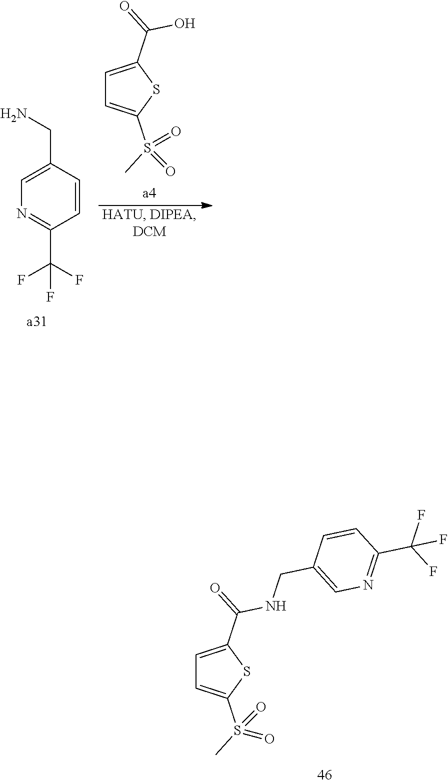

To a stirred solution of a4 (300 mg, 1.45 mmol) and a31 (307.46 mg, 1.75 mmol) in DCM (10 mL) was added DIPEA (0.51 mL, 2.91 mmol) and HATU (829.63 mg, 2.18 mmol) at 0° C. and then stirring was continued for 1 h at the same temperature. The reaction mixture was quenched with water (10 mL) and extracted with DCM (2×25 mL). The combined organic layers were washed with water (20 mL), brine (20 mL), dried over anhydrous Na₂SO₄ and concentrated under reduced pressure. The obtained crude product was purified by combi-flash chromatography on silica gel using 0-40% EtOAc in hexane as an eluent to afford 46 (253 mg, 0.69 mmol, 47% yield), as a solid. HPLC: Rt 6.612 min, 99.62%; Column: X Select CSH C18(150×4.6)mm, 3.5μ; Mobile phase A: 0.1% FA in Water:ACN (95:05); Mobile phase B: Acetonitrile; Gradient Program: T/B %: 0.01/5, 1/5, 8/100, 12/100, 14/5, 18/5; Flow rate: 1.2 ml/min. LCMS: 365.0 (M+H), Rt 1.694 min, Mobile Phase: A: 0.025% FA in Water, B: ACN; T/B %: 0.01/2, 0.2/2, 2.2/98, 3/98, 3.2/2, 4/2; Flow rate: 1.2 ml/min(Gradient); Column Oven temperature: 50° C. ¹H NMR (400 MHz, DMSO-d6) δ 9.52 (t, 1H), 8.75 (d, 1H), 8.01 (dd, 1H), 7.92-7.81 (m, 3H), 4.61 (d, 2H), 3.38 (s, 3H).

### Example 48. Synthesis of 5-(cyclopropylsulfonyl)-N-(4-(trifluoromethyl)benzyl)thiophene-2-carboxamide (Compound 47)

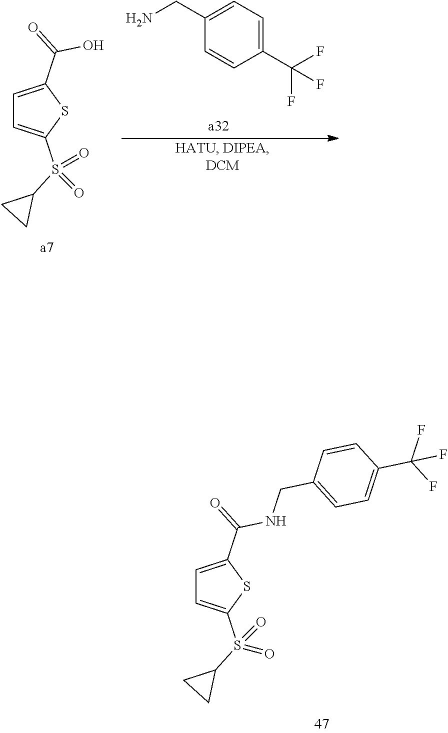 [SMILES: O=C(O)c1ccc(S(=O)(=O)C2CC2)s1 ; NCc1ccc(C(F)(F)F)cc1 ; "HATU, DIPEA,DCM" ; O=C(NCc1ccc(C(F)(F)F)cc1)c1ccc(S(=O)(=O)C2CC2)s1]

To a stirred reaction mixture of a7 (250 mg, 1.08 mmol) and a32 (226.21 mg, 1.29 mmol) in DCM (10 mL) was added HATU (818.47 mg, 2.15 mmol) followed by DIPEA (0.56 mL, 3.23 mmol) at 0° C. and then stirring was continued at same temperature further 2 h. The reaction mixture was quenched with water and the aqueous layer was extracted with DCM (2×25 mL). The combined organic layers were washed with brine (20 mL), dried over anhydrous Na₂SO₄, filtered and concentrated under reduced pressure. The obtained crude product was purified by flash chromatography on silica gel (100-200 mesh) using EtOAc in heptane=0% to 60% as an eluent to afford 47 (348 mg, 0.8803 mmol, 81% yield) as a solid. HPLC: Rt 7.264 min, 98.52%; Column: X-Select CSH C18 (4.6*150) mm 3.5u; Mobile Phase: A—0.1% Formic acid in water:Acetonitrile (95:05); B—Acetonitrile; Flow Rate: 1.0. mL/minute; Gradient program: Time (min)/B Conc.: 0.01/10, 6.0/90, 10.0/100, 12.0/100, 14/10, 18.0/10. LCMS: 388.05 (M−H), Rt 1.873 min, Column: X-Bridge BEH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. ¹H NMR (400 MHz, DMSO-d6) δ 9.53 (t, 1H), 7.90 (d, 1H), 7.82 (d, 1H), 7.72 (d, 2H), 7.55 (br d, 2H), 4.57 (br d, 2H), 3.07-2.99 (m, 1H), 1.22-1.09 (m, 4H).

### Example 49. Synthesis of 5-(methylsulfonyl)-N-((5-(trifluoromethyl)pyridin-2-yl)methyl)thiophene-2-carboxamide (Compound 48)

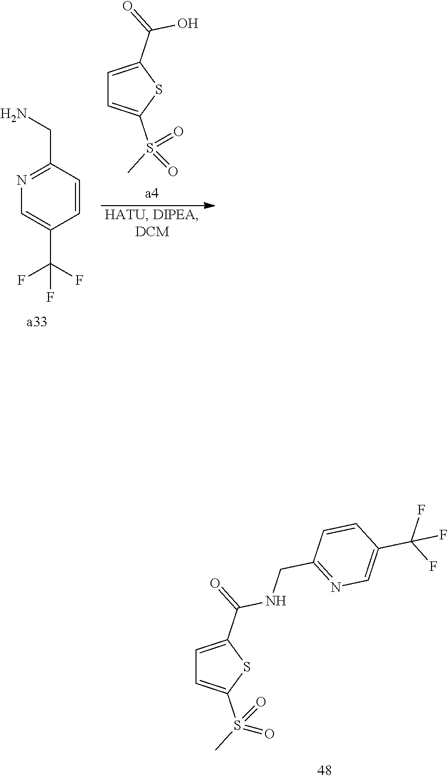 [SMILES: NCc1ccc(C(F)(F)F)cn1 ; CS(=O)(=O)c1ccc(C(=O)O)s1 ; "HATU, DIPEA,DCM" ; CS(=O)(=O)c1ccc(C(=O)NCc2ccc(C(F)(F)F)cn2)s1]

To a stirred reaction mixture of a4 (250 mg, 1.21 mmol) and a33 (255 mg, 1.45 mmol) in DCM (10 mL) was added HATU (921.81 mg, 2.42 mmol) followed by DIPEA (0.63 mL, 3.64 mmol) at 0° C. and then stirring was continued at same temperature further 2 h. The reaction mixture was quenched with water (20 mL) and the aqueous layer was extracted with DCM (2×25 mL). The combined organic layers were washed with brine (20 mL), dried over anhydrous Na₂SO₄, filtered and concentrated under reduced pressure. The obtained crude product was then purified by flash chromatography on silica gel (100-200 mesh) using EtOAc in heptane=0% to 80% as an eluent to afford 48 (437 mg, 1.19 mmol, 97% yield) as a solid. HPLC: Rt 6.106 min, 98.81%; Column: X-Select CSH C18 (4.6*150) mm 3.5u; Mobile Phase: A—0.1% Formic acid in water:Acetonitrile (95:05); B-Acetonitrile; Flow Rate: 1.0. mL/minute; Gradient program: Time (min)/B Conc.: 0.01/10, 6.0/90, 10.0/100, 12.0/100, 14/10, 18.0/10. LCMS: 364.90 (M+H), Rt 1.713 min, Column: X-Bridge BEH C18 (50*3) mm 2.5u; Mobile Phase: A: 2.5 mM Ammonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. 1H NMR (400 MHz, DMSO-d6) ¹H NMR (400 MHz, DMSO-d₆) δ=9.62 (br t, 1H), 8.93 (s, 1H), 8.20 (dd, 1H), 7.92 (d, 1H), 7.85 (d, 1H), 7.59 (d, 1H), 4.67 (br d, 2H), 3.40 (s, 3H).

### Example 50. Synthesis of 5-(ethylsulfonyl)-N-(4-(trifluoromethyl)benzyl)thiophene-2-carboxamide (Compound 49)

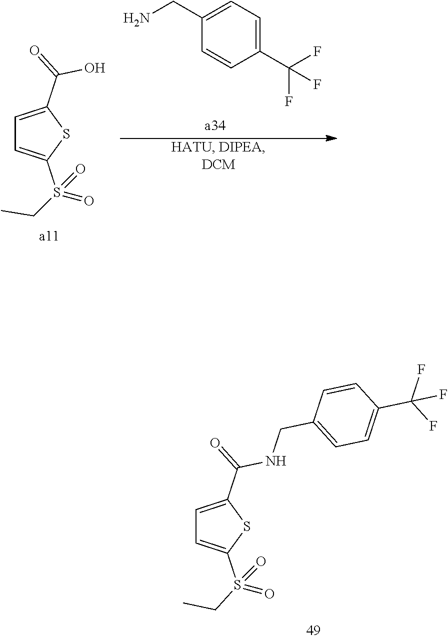 [SMILES: CCS(=O)(=O)c1ccc(C(=O)O)s1 ; NCc1ccc(C(F)(F)F)cc1 ; "HATU, DIPEA,DCM" ; CCS(=O)(=O)c1ccc(C(=O)NCc2ccc(C(F)(F)F)cc2)s1]

To a stirred solution of a11 (180 mg, 0.8200 mmol) in DCM (3 mL) was added a34 (143.13 mg, 0.82 mmol) and HATU (310.72 mg, 0.82 mmol) followed by DIPEA (0.28 mL, 1.63 mmol) at 0° C., stirring was continued further for 1 h at 0° C. The reaction mixture was quenched with water (10 mL) and extracted with DCM (2×25 mL). The combined organic layers were washed with water (10 mL), brine (10 mL), dried over anhydrous Na₂SO₄ and concentrated under reduced pressure. The obtained crude product was purified by Combi-Flash chromatography on silica gel using 0-40% EtOAc in hexane as an eluent to afford 49 (70 mg, 0.18 mmol, 22% yield) as a solid. HPLC: Rt 7.879 min, 98.28%; Column: X-Select CSH C18 (4.6*150) mm 5u; Mobile Phase: A—0.1% TFA in water; B—Acetonitrile; Inj Volume; 5.0 μL; Flow Rate: 1.2. mL/minute; Gradient program: Time (min)/B Conc.: 0.01/5, 1.0/5, 8.0/100, 12.0/100, 14.0/5, 18.0/5. LCMS: 376.2 (M−H), Rt 2.037 min, Column: X-Bridge BEH C18 (50*3) mm 2.5u; Mobile Phase: A: 2.5 mMAmmonium Bicarbonate in water; B: Acetonitrile; Inj Volume: 2 μL, Flow Rate: 1.2 mL/minute; Column oven temp. 50° C.; Gradient program: 0% B to 98% B in 2.0 minute, hold till 3.0 min, at 3.2 min B conc is 0% up to 4.0 min. ¹H NMR (400 MHz, DMSO-d₆) δ 9.50 (t, 1H), 7.90 (d, 1H), 7.81 (d, 1H), 7.71 (d, 2H), 7.54 (d, 2H), 4.56 (d, 2H), 3.44 (q, 2H), 1.18 (t, 3H).

### Example 51. Synthesis of 5-(isopropylsulfonyl)-N-(4-(trifluoromethyl)benzyl)thiophene-2-carboxamide (Compound 50)

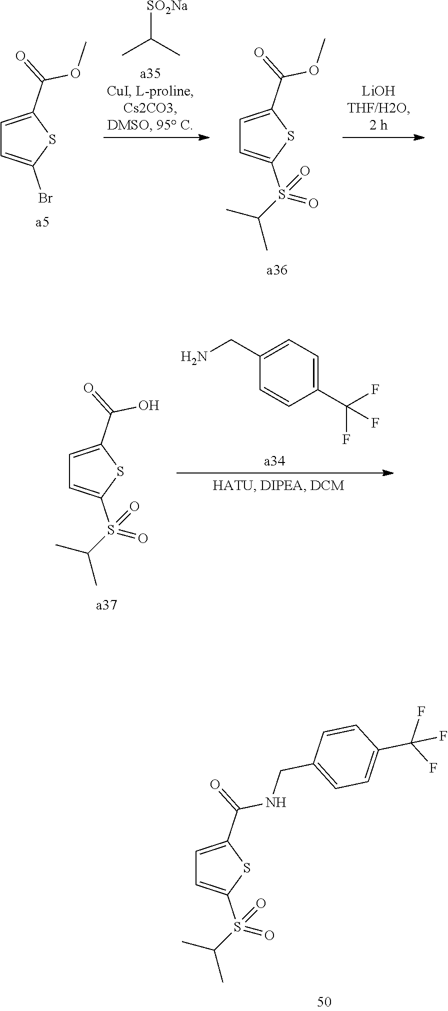

### Synthesis of methyl methyl 5-(isopropylsulfonyl)thiophene-2-carboxylate (a36)

To a stirred solution of a5 (1 g, 4.52 mmol) in DMSO (30 mL) were added a35 (1 g, 7.8 mmol), L-proline (208.31 mg, 1.81 mmol), copper iodide (343.78 mg, 1.81 mmol) followed by Cs₂CO₃ (294.77 mg, 0.9000 mmol) at room temperature and then stirring was continued further 16 h at 95° C. The reaction mixture was allowed to cool to room temperature and then diluted with water (30 mL) and EtOAC (30 mL), the obtained crude was filtered through celite pad. The filtrate was separated and aqueous layer was washed with EtOAc (30 mL), the combined organic layers were dried over anhydrous Na₂SO₄, filtered, and concentrated under vacuo to obtain a crude residue. The obtained crude was then purified by flash column chromatography eluting 20% EtOAc in hexane to afford a36 (100 mg, 0.40 mmol, 9% yield) as a solid.

### Synthesis of 5-(isopropylsulfonyl)thiophene-2-carboxylic acid (a37)

To stirred solution of a36 (100 mg, 0.40 mmol) in THE (4 mL) and water (1 mL) was added LiOH (48 mg, 1.2 mmol) at room temperature and then stirring was continued at same temperature for 2 h. The reaction mixture was acidified with 2M HCl and extracted with ethyl acetate (2×10 mL). The combined organic layers were dried over Na₂SO₄ and concentrated under reduced pressure to afford a37 (75 mg, 0.32 mmol, 79% yield), which was used in the next step without further purification.

### Synthesis of 5-(isopropylsulfonyl)-N-(4-(trifluoromethyl)benzyl)thiophene-2-carboxamide (Compound 50)

To a stirred reaction mixture of a37 (70 mg, 0.3 mmol) and a34 (62.8 mg, 0.36 mmol) in DCM (5 mL) were added HATU (227.2 mg, 0.6 mmol) followed by DIPEA (0.16 mL, 0.90 mmol) at 0° C. and then stirring was continued at same temperature further 2 h. The reaction mixture was quenched with water (10 mL) and the aqueous layer was extracted with DCM (2×25 mL). The combined organic layers were washed with brine (20 mL), dried over anhydrous Na₂SO₄, filtered and concentrated under reduced pressure. The obtained crude product was then purified by flash chromatography on silica gel (100-200 mesh) using EtOAc in heptane=0% to 60% as an eluent to afford 50 (38 mg, 0.095 mmol, 31% yield) as a solid. HPLC: Rt 10.47 min, 98.21%; Column: X SELECT; Program: T/B %: 0.01/20, 12/90, 16/90; Flow: 1.0 mL/min; Diluent: ACN:WATER (80:20). LCMS: 391.90 (M+H), Rt 2.055 min, Column: X-Select CSH C18 (3.0*50) mm 2.5 um; Mobile Phase: A: 0.05% Formic acid in water:ACN (95:05); B: ACN; Inj Volume: 2.0 μL; Flow Rate: 1.2. mL/minute; Column oven Temp: 50° C.; Gradient program: 0% B to 98% B in 2.0 minute, Hold till 3.0 min, At 3.2 min B conc is 0% up to 4.0 min. ¹H NMR (400 MHz, DMSO-d6) δ 9.51 (t, 1H), 7.92 (d, 1H), 7.79 (d, 1H), 7.71 (d, 2H), 7.55 (d, 2H), 4.56 (d, 2H), 3.53 (quin, 1H), 1.23 (d, 6H).

### Example 52. Synthesis of N-(4-(difluoromethyl)benzyl)-5-(methylsulfonyl)thiophene-2-carboxamide (Compound 51)

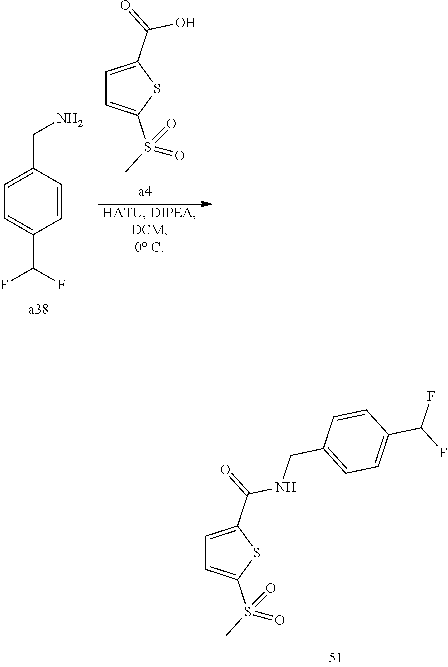

To a stirred solution of a4 (200 mg, 0.97 mmol) and a38 (167.65.mg, 1.07 mmol) in DCM (5 mL) was added HATU (553.09 mg, 1.45 mmol) followed by DIPEA (0.51 mL, 2.91 mmol) at 0° C. and then stirring was continued at same temperature for 1 h. The reaction mixture was quenched with water (10 mL) and extracted with EtOAc (2×25 mL). The combined organic layers were washed with water (20 mL), brine (20 mL), dried over anhydrous Na₂SO₄ and concentrated under reduced pressure. The obtained crude product was purified by flash chromatography on silica gel using 30-40% EtOAc in heptane as an eluent to afford 51 (180 mg, 0.52 mmol, 53% yield), as a solid. HPLC: Rt 7.047 mi, 99.416%; Column: X Select CSH C18(150×4.6)mm, 3.5μ; Mobile phase A 0.1% FA in Water:ACN (95:05); Mobile phase B Acetonitrile; Gradient Programme: T/B %: 0.01/5, 1/5, 8/100, 12/100, 14/5, 18/5; Flow rate: 1.2 ml/min. LCMS: 345.80 (M+H), Rt 1.692 min, Column: X-Select CSH (3.0*50) mm 2.5u; Mobile Phase: A: 0.05% Formic acid in water:ACN (95:5); B: ACN; Inj Volume: 2.0 μL; Flow Rate: 1.2. mL/minute; Column oven temperature: 50° C.; Gradient program: 0% B to 98% B in 2.0 min, hold till 3.0 min, at 3.2 min B conc is 0% up to 4.0 min. ¹H NMR (400 MHz, DMSO-d₆) δ 9.47 (t, 1H), 7.89 (d, 1H), 7.84 (d, 1H), 7.56 (d, 2H), 7.47 (d, 2H), 7.03 (t, 1H), 4.55 (d, 2H), 3.40 (s, 3H).

### Example 53. Synthesis of 5-((2-methoxyethyl)sulfonyl)-N-(4-(trifluoromethyl)benzyl)thiophene-2-carboxamide (Compound 52)

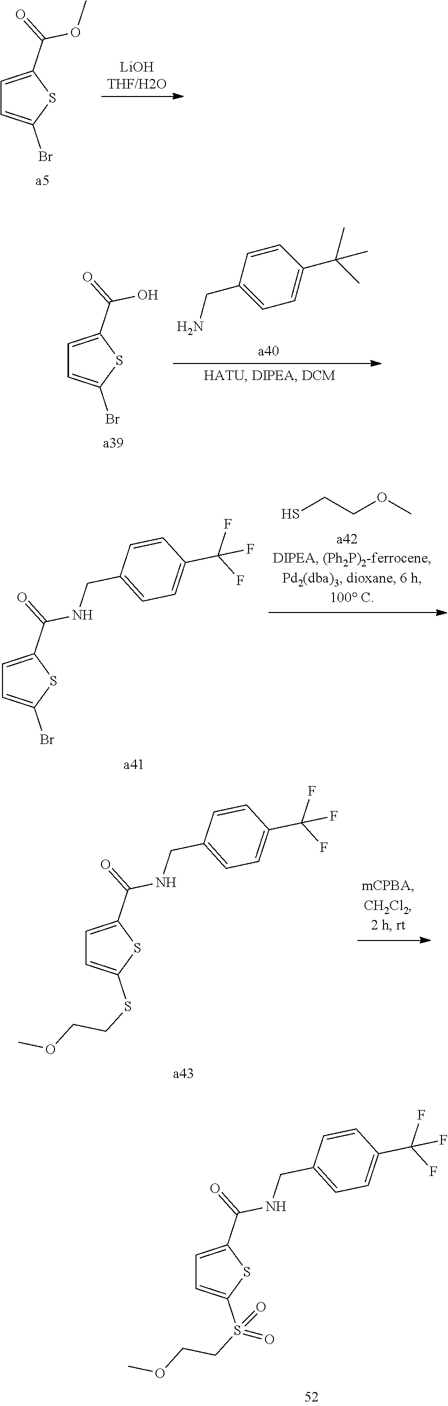

### Synthesis of 5-bromothiophene-2-carboxylic acid (a39)

To a stirred solution of a5 (5 g, 22.62 mmol) in THE (45 mL) and water (20 mL) was added LiOH·H₂O (1.9 g, 45.23 mmol). The reaction mixture was stirred at room temperature for 2 h. The reaction mixture was concentrated under reduced pressure, diluted with water (20 mL). The aqueous layer was washed with DCM (20 mL), acidified using 2N HCl (20 mL). The obtained precipitate was filtered and dried to afford a39 (4.9 g, 20.826 mmol, 92% yield), as a solid.

### Synthesis of 5-bromo-N-(4-(trifluoromethyl)benzyl)thiophene-2-carboxamide (a41)

To a stirred solution of a39 (1.5 g, 7.24 mmol) and a40 (1.52 g, 8.69 mmol) in DCM (50 mL) was added HATU (4.13 g, 10.87 mmol) followed by DIPEA (2.52 mL, 14.49 mmol) at 0° C., then stirring was continued further for 1 h at 0° C. The reaction mixture was quenched with water (20 mL) and extracted with DCM (2×50 mL). The combined organic layers were washed with brine (20 mL), dried over anhydrous Na₂SO₄ and concentrated under reduced pressure. The obtained crude was purified by Combi-Flash column chromatography (100-200 silica gel) by eluting 0-40% EtOAc in hexanes to afford a41 (2.32 g, 6.37 mmol, 87% yield), as a solid.

### Synthesis of 5-((2-methoxyethyl)thio)-N-(4-(trifluoromethyl)benzyl)thiophene-2-carboxamide (a43)

To a stirred solution of a41 (600 mg, 1.65 mmol) in 1,4-dioxane (10 mL) was added a42 (303.67 mg, 3.3 mmol) and DIPEA (0.86 mL, 4.94 mmol). The reaction mixture was degassed under N₂ atmosphere for 20 min followed by the addition of tris(dibenzylidene-acetone)dipalladium(0) (150.87 mg, 0.16 mmol) and 1,1′-ferrocenediyl-bis(diphenylphosphine (182.67 mg, 0.33 mmol). The reaction mixture was microwaved at 110° C. for 1 h. The reaction mixture was diluted with water (10 mL) and extracted with EtOAc (2×20 mL). The combined organic layers were dried over Na₂SO₄ and concentrated under reduced pressure. The obtained crude was then purified by combi-flash using 10-20% EtOAc/hexane as an eluent to afford a43 (510 mg, 1.3584 mmol, 82% yield) as a solid.

### Synthesis of 5-((2-methoxyethyl)sulfonyl)-N-(4-(trifluoromethyl)benzyl)thiophene-2-carboxamide (Compound 52)

To a stirred solution of a43 (400 mg, 1.07 mmol) in DCM (10 mL) was added m-CPBA (551.59 mg, 3.2 mmol) portion wise at 0° C. and then stirred at room temperature for 2 h. The reaction mixture was quenched with water (20 mL) and the aqueous layer was extracted with DCM (2×25 mL). The combined organic layers were washed with brine (20 mL), dried over anhydrous Na₂SO₄, filtered and concentrated under reduced pressure. The obtained crude product was purified by Combi-Flash column chromatography (100-200 silica gel) by eluting 0-40% EtOAc in hexanes to afford 52 (303.33 mg, 0.7428 mmol, 69% yield) as a solid. HPLC: Rt 9.378 min, 99.77%; Column: X Select CSH C18(150×4.6)mm, 3.5μ; Mobile phase A: 5 mM NH₄HCO₃; Mobile phase B: Acetonitrile; Gradient Programme: T/B %: 0.01/20, 12/90, 16/90; Flow rate: 1 mL/min Dilutant: ACN:Water (20:80). LCMS: 406.20 (M−H), Rt 2.242 min, Column: X-Bridge BEH C18 (50*3) mm 2.5u; Mobile Phase: A: 2.5 mMAmmonium Bicarbonate in water; B: Acetonitrile; Inj Volume: 2 μL, Flow Rate: 1.2 mL/minute; Column oven temp. 45° C.; Gradient program: 0% B to 98% B in 2.0 minute, hold till 3.0 min, at 3.2 min B conc is 0% up to 4.0 min. ¹H NMR (400 MHz, DMSO-d6): δ 9.49 (t, 1H), 7.87 (d, 1H), 7.80 (d, 1H), 7.71 (d, 2H), 7.54 (d, 2H), 4.56 (d, 2H), 3.78-3.71 (m, 2H), 3.70-3.64 (m, 2H), 3.15 (s, 3H).

### Example 54. Synthesis of N-(4-(difluoromethyl)-2-methylbenzyl)-5-(methylsulfonyl)thiophene-2-carboxamide (Compound 53)

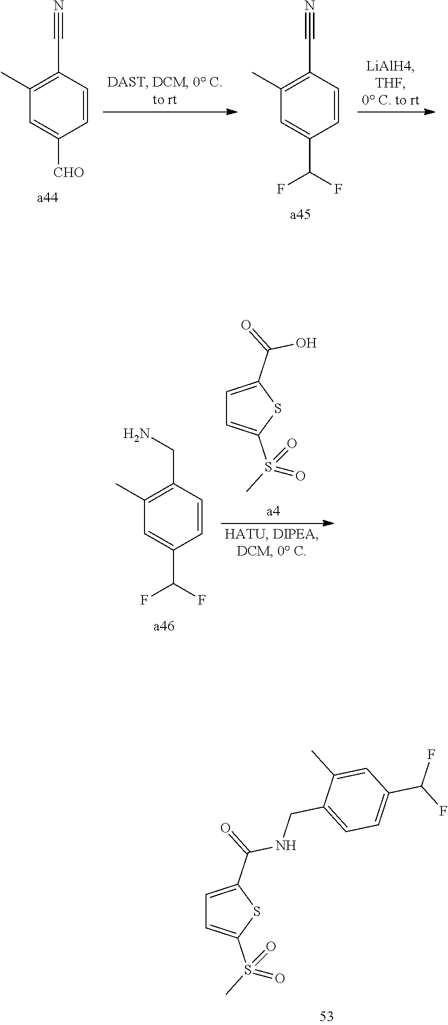

### Synthesis of 4-(difluoromethyl)-2-methylbenzonitrile (a45)

To a stirred solution of a44 (400 mg, 2.76 mmol) in DCM (4 mL) was added DAST (1.84 mL, 13.78 mmol) at 0° C. and then stirring was continued at room temperature for 16 h. The reaction mixture was poured into ice cold water and extracted with ethyl acetate (2×20 mL). The combined organic layers were washed with NaHCO₃ solution, brine solution (20 mL), dried over anhydrous Na₂SO₄ and concentrated under reduced pressure. The obtained crude was then purified by flash column chromatography eluting with 8% EtOAc in isohexane to afford a45 (410 mg, 1.96 mmol, 71% yield) as a liquid.

### Synthesis of (4-(difluoromethyl)-2-methylphenyl)methanamine (a46)

To a stirred solution of a45 (380 mg, 1.82 mmol) in THF (5 mL) was added LAH (2.73 mL, 5.46 mmol) dropwise at 0° C. The resulting reaction mixture was stirred at room temperature for 3 h. The reaction mixture was quenched with 15% NaOH solution (5 mL) and filtered through celite. The filtrate was extracted with EtOAc (2×15 mL). The combined organic layers were washed with water (20 mL), brine (20 mL), dried over anhydrous MgSO₄ and concentrated under reduced pressure to afford a46 (310 mg, 0.52 mmol, 29% yield) as a solid.

### Synthesis of N-(4-(difluoromethyl)-2-methylbenzyl)-5-(methylsulfonyl)thiophene-2-carboxamide (Compound 53)

To a stirred solution of a46 (300 mg, 0.510 mmol) and a4 (83 mg, 0.41 mmol) in DCM (3 mL) was added DIPEA (0.27 mL, 1.52 mmol) and HATU (232 mg, 0.61 mmol) at 0° C. and then stirring was continued at same temperature for 1 h. The reaction mixture was quenched with water (10 mL) and extracted with ethyl acetate (2×10 mL). The combined organic layers were washed with water (20 mL), brine (20 mL), dried over anhydrous MgSO₄ and concentrated under reduced pressure. The obtained crude product was then purified by combi-flash chromatography on silica gel using 30% EtOAc in hexane as an eluent to afford 53 (75 mg, 0.20 mmol, 40% yield) as a solid. HPLC: Rt 6.672 min, 97.783%; Method File: HPLC-FORMIC ACID-XSELECT-10-90-100.lcm; Column: X-Select CSH C18 (4.6*150) mm 3.5u; Mobile Phase: A—0.1% Formic acid in water:Acetonitrile (95:05); B-Acetonitrile; Flow Rate: 1.0. mL/minute; Gradient program: Time (min)/B Conc: 0.01/10, 6.0/90, 10.0/100, 12.0/100, 14/10, 18.0/10. LCMS: 359.90 (M+H), Rt 1.716 min, Column: X-Select CSH (3.0*50) mm 2.5u; Mobile Phase: A: 0.05% Formic acid in water:ACN (95:5); B: ACN; Inj Volume: 2.0 μL; Flow Rate: 1.2. mL/minute; Column oven temperature: 50° C.; Gradient program: 0% B to 98% B in 2.0 min, hold till 3.0 min, at 3.2 min B conc is 0% up to 4.0 min. ¹H NMR (400 MHz, DMSO-d6): δ 9.33 (t, 1H), 7.91 (d, 1H), 7.83 (d, 1H), 7.40-7.37 (m, 3H), 7.13-6.83 (m, 1H), 4.50 (d, 2H), 3.39 (s, 3H), 2.38 (s, 3H).

### Example 55. Synthesis of N-((4-methyl-6-(trifluoromethyl)pyridin-3-yl)methyl)-5-(methylsulfonyl)thiophene-2-carboxamide (Compound 54)

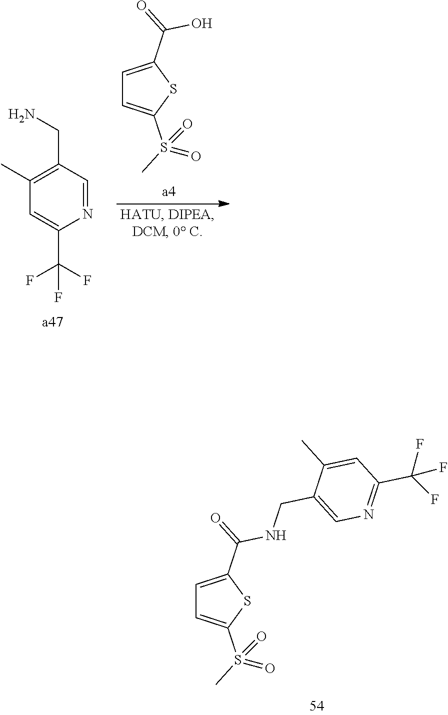 [SMILES: Cc1cc(C(F)(F)F)ncc1CN ; CS(=O)(=O)c1ccc(C(=O)O)s1 ; Cc1cc(C(F)(F)F)ncc1CNC(=O)c1ccc(S(C)(=O)=O)s1]

To a stirred solution of a4 (117.13 mg, 0.57 mmol) and a47 (90 mg, 0.47 mmol) in DCM (3 mL) was added DIPEA (0.25 mL, 1.42 mmol) and HATU (269.92 mg, 0.71 mmol) at 0° C. and then stirring was continued at same temperature for 1 h. The reaction mixture was quenched with water (10 mL) and extracted with ethyl acetate (2×10 mL). The combined organic layers were washed with water (20 mL), brine (20 mL), dried over anhydrous MgSO₄ and concentrated under reduced pressure. The obtained crude product was then purified by combi-flash chromatography on silica gel using 0-40% EtOAc in hexane as an eluent to afford 54 (80 mg, 0.2067 mmol, 43% yield) as a solid. HPLC: Rt 6.959 min, 97.8%; Column: X Select CSH C18(150×4.6)mm, 3.5μ; Mobile phase A: 0.1% FA in Water:ACN (95:05); Mobile phase B: Acetonitrile; Gradient Programme: TB %: 0.01/5, 1/5, 8/100, 12/100, 14/5, 18/5; Flow rate: 1.2 ml/min. LCMS: 378.75 (M+H), Rt 1.668 min; Column: X-Select CSH (3.0*50) mm 2.5u; Mobile Phase: A: 0.05% Formic acid in water:ACN (95:5); B: ACN; Inj Volume: 2.0 μL; Flow Rate: 1.2. mL/minute; Column oven temperature: 50° C. Gradient program: 0% B to 98% B in 2.0 min, hold till 3.0 min, at 3.2 min B conc is 0% up. ¹H NMR (400 MHz, DMSO-d6): δ 9.35 (br t, 1H), 8.60 (s, 1H), 7.86 (d, 1H), 7.82 (d, 1H), 7.78 (s, 1H), 4.58 (br d, 2H), 3.38 (s, 3H), 2.46 (s, 3H).

### Example 55. Efficacy of Exemplary Compounds in the Inhibition of KCNT1

### KCNT1—Patch Clamp Assay

Inhibition of KCNT1 (KNa1.1, Slack) was evaluated using a tetracycline inducible cell line (HEK-TREX). Currents were recorded using the SyncroPatch 384PE automated, patch clamp system. Pulse generation and data collection were performed with PatchController384 V1.3.0 and DataController384 V1.2.1 (Nanion Technologies). The access resistance and apparent membrane capacitance were estimated using built-in protocols. Current were recorded in perforated patch mode (10 μM escin) from a population of cells. The cells were lifted, triturated, and resuspended at 800,000 cells/ml. The cells were allowed to recover in the cell hotel prior to experimentation. Currents were recorded at room temperature. The external solution contained the following (in mM): NaCl 105, NMDG 40, KCl 4, MgCl₂ 1, CaCl₂ 5 and HEPES 10 (pH=7.4, Osmolarity ˜300 mOsm). The extracellular solution was used as the wash, reference and compound delivery solution. The internal solution contained the following (in mM): NaCl 70, KF 70, KCl 10, EGTA 5, HEPES 5 and Escin 0.01 (pH=7.2, Osmolarity ˜295 mOsm). Escin is made at a 5 mM stock in water, aliquoted, and stored at −20° C. The compound plate was created at 2× concentrated in the extracellular solution. The compound was diluted to 1:2 when added to the recording well. The amount of DMSO in the extracellular solution was held constant at the level used for the highest tested concentration. A holding potential of −80 mV with a 100 ms step to 0 mV was used. Mean current was measured during the step to 0 mV. 100 μM Bepridil was used to completely inhibit KCNT1 current to allow for offline subtraction of non-KCNT1 current. The average mean current from 3 sweeps was calculated and the % inhibition of each compound was calculated. The % Inhibition as a function of the compound concentration was fit with a Hill equation to derive IC₅₀, slope, min and max parameters. If KCNT1 inhibition was less than 50% at the highest tested concentration or if an IC₅₀ could not be calculated, then a percent inhibition was reported in place of the IC₅₀.

Results from this assay are summarized in Table 1 below. In this table, “A” indicates IC₅₀ of less than or equal to 1 μM; “B” indicates inhibition of between 1 μM to 20 μM; and “C” indicates inhibition of greater than or equal to 20 μM.

TABLE 1

| Patent Compound | KCNT1-WT |
|---|---|
| No. | IC₅₀ (μM) |
| 1 | B |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | B |
| 18 | A |
| 19 | A |
| 20 | A |
| 21 | A |
| 22 | A |
| 23 | C |
| 24 | A |
| 25 | C |
| 26 | A |
| 27 | C |
| 28 | A |
| 29 | A |
| 30 | C |
| 31 | A |
| 32 | A |
| 33 | A |
| 34 | B |
| 35 | B |
| 36 | A |
| 37 | A |
| 38 | B |
| 39 | B |
| 40 | C |
| 41 | C |
| 42 | A |
| 43 | A |
| 44 | A |
| 45 | A |
| 46 | B |
| 47 | A |
| 48 | B |
| 49 | A |
| 50 | A |
| 51 | A |
| 52 | A |
| 53 | A |
| 54 | B |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A pharmaceutical composition comprising a compound of Formula A: 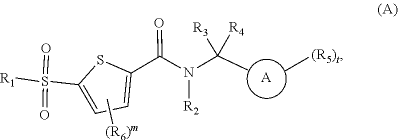
or a pharmaceutically acceptable salt thereof, wherein
A is phenyl or pyridyl;
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₂ is hydrogen or C₁₋₆alkyl;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
R₆ is C₁₋₆alkyl or C₁₋₆alkoxy;
t is 0, 1, 2, 3, or 4; and
m is 0, 1, or 2;
and a pharmaceutically acceptable carrier.

2. A pharmaceutical composition comprising a compound of Formula A-1, Formula A-2, or Formula A-3: 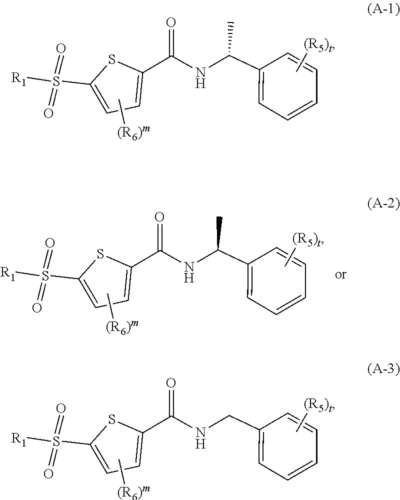
or a pharmaceutically acceptable salt thereof, wherein
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
R₆ is C₁₋₆alkyl or C₁₋₆alkoxy;
t is 0, 1, 2, 3, or 4; and
m is 0, 1, or 2
and a pharmaceutically acceptable carrier.

3. The pharmaceutical composition of claim 1 or 2, wherein R₁ is C₁₋₆alkyl or —NHRₐ.

4. The pharmaceutical composition of any one of claims 1-3, wherein R₁ is C₁₋₆alkyl.

5. The pharmaceutical composition of any one of claims 1-4, wherein R₁ is methyl, ethyl, or isopropyl.

6. The pharmaceutical composition of any one of claims 1-3, wherein R₁ is —NHRₐ.

7. The pharmaceutical composition of claim 6, wherein Rₐ is C₁₋₆alkyl.

8. The pharmaceutical composition of claim 6 or 7, wherein Rₐ is methyl.

9. The pharmaceutical composition of any one of claims 1 and 3-8, wherein R₃ is hydrogen.

10. The pharmaceutical composition of any one of claims 1 and 3-9, wherein R₄ is hydrogen or methyl.

11. The pharmaceutical composition of any one of claims 1-10, wherein R₅ is each independently selected from the group consisting of halogen, cyano, —OH, C₁₋₆alkyl, C₁₋₆haloalkyl, C₁₋₆alkoxy, and C₃₋₈cycloalkyl, wherein the C₁₋₆alkyl or C₃₋₈cycloalkyl is optionally substituted with halogen, cyano, or C₁₋₆haloalkyl.

12. The pharmaceutical composition of any one of claims 1-11, wherein R₅ is each independently selected from the group consisting of chloro, fluoro, bromo, cyano, —OH, methyl, ethyl, isopropyl, tert-butyl, —CHCF₂, —CF₃, —OCH₃, —OCH₂CH₃, —OCH(CH₃)₂, —OCH₂CF₃, and cyclopropyl optionally substituted with —CF₃.

13. The pharmaceutical composition of any one of claims 1-12, wherein t is 1 or 2.

14. The pharmaceutical composition of any one of claims 1-13, wherein m is 0.

15. A compound of Formula II: 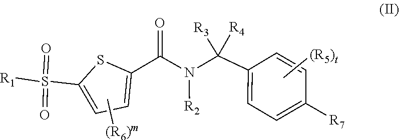
or a pharmaceutically acceptable salt thereof, wherein
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₂ is hydrogen;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆ haloalkyl, or C₁₋₆alkoxy;
R₆ is C₁₋₆alkyl or C₁₋₆alkoxy;
R₇ is selected from the group consisting of halogen, cyano, —NR_{c}R_{d}, C₁₋₆alkyl, C₁₋₆haloalkyl, C₁₋₆alkoxy, C₁₋₆haloalkoxy, and C₃₋₈cycloalkyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
t is 0, 1, 2, or 3; and
m is 0, 1, or 2.

16. A compound of Formula II-b: 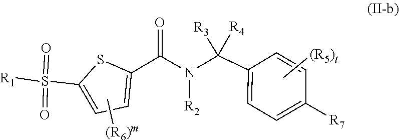
or a pharmaceutically acceptable salt thereof, wherein
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is C₁₋₆alkyl;
R₂ is hydrogen;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, —C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R₆ is C₁₋₆alkyl or C₁₋₆alkoxy;
R₇ is selected from the group consisting of halogen, cyano, —NR_{c}R_{d}, C₁₋₆alkyl, C₁₋₆haloalkyl, C₁₋₆alkoxy, C₁₋₆haloalkoxy, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
t is 0, 1, 2, or 3; and
m is 0, 1, or 2.

17. The compound of claim 15 or 16, wherein R₁ is C₁₋₆alkyl or —NHRₐ.

18. The compound of any one of claims 15-17, wherein R₁ is C₁₋₆alkyl.

19. The compound of any one of claims 15-18, wherein R₁ is methyl, ethyl, or isopropyl.

20. The compound of any one of claims 15-19, wherein R₁ is methyl.

21. The compound of any one of claims 15-17, wherein R₁ is —NHRₐ.

22. The compound of claim 21, wherein Rₐ is C₁₋₆alkyl.

23. The compound of claim 21 or 22, wherein Rₐ is methyl.

24. The compound of claim 15 or 16, wherein R₁ is C₃₋₈cycloalkyl.

25. The compound of any one of claims 15, 16, and 24, wherein R₁ is cyclopropyl.

26. The compound of any one of claims 15-25, wherein R₃ is hydrogen.

27. The compound of any one of claims 15-26, wherein R₄ is hydrogen or methyl.

28. The compound of any one of claims 15-27, wherein R₃ and R₄ are hydrogen.

29. The compound of any one of claims 15-28, wherein R₅ is each independently selected from the group consisting of halogen, cyano, —OH, C₁₋₆alkyl, C₁₋₆haloalkyl, C₁₋₆alkoxy, and C₃₋₈cycloalkyl, wherein the C₁₋₆alkyl or C₃₋₈cycloalkyl is optionally substituted with halogen, cyano, or C₁₋₆haloalkyl.

30. The compound of any one of claims 15-29, wherein R₅ is each independently selected from the group consisting of chloro, fluoro, bromo, cyano, —OH, methyl, ethyl, isopropyl, tert-butyl, —CHCF₂, —CF₃, —OCH₃, —OCH₂CH₃, —OCH(CH₃)₂, —OCH₂CF₃, and cyclopropyl optionally substituted with —CF₃.

31. The compound of any one of claims 15-28, wherein R₅ is each independently selected from the group consisting of halogen, —OH, C₁₋₆alkyl, C₁₋₆haloalkyl, C₁₋₆alkoxy, and C₃₋₈cycloalkyl.

32. The compound of any one of claims 15-28 and 31, wherein R₅ is each independently selected from the group consisting of chloro, fluoro, bromo, —OH, methyl, —CF₃, —OCH₃, and cyclopropyl.

33. The compound of any one of claims 15-32, wherein R₇ is selected from the group consisting of halogen, cyano, C₁₋₆alkyl optionally substituted with cyano, C₁₋₆haloalkyl, C₁₋₆alkoxy, C₁₋₆haloalkoxy, and C₃₋₈cycloalkyl optionally substituted with C₁₋₆haloalkyl.

34. The compound of any one of claims 15-33, wherein R₇ is selected from the group consisting of chloro, bromo, cyano, methyl, ethyl, isopropyl, tert-butyl, —CHCF₂, —CF₃, —OCH₂CH₃, —OCH₂CF₃, and cyclopropyl optionally substituted with —CF₃.

35. The compound of any one of claims 15-32, wherein R₇ is 4-8 membered heterocyclyl.

36. The compound of claim 35, wherein the 4-8 membered heterocyclyl comprises one nitrogen.

37. The compound of any one of claims 15-36, wherein t is 1 or 2.

38. The compound of any one of claims 15-36, wherein t is 0.

39. The compound of any one of claims 15-37, wherein t is 1.

40. The compound of any one of claims 15-37, wherein t is 2.

41. The compound of any one of claims 15-40, wherein m is 0.

42. The compound of claim 15, wherein the compound selected from the group consisting of: 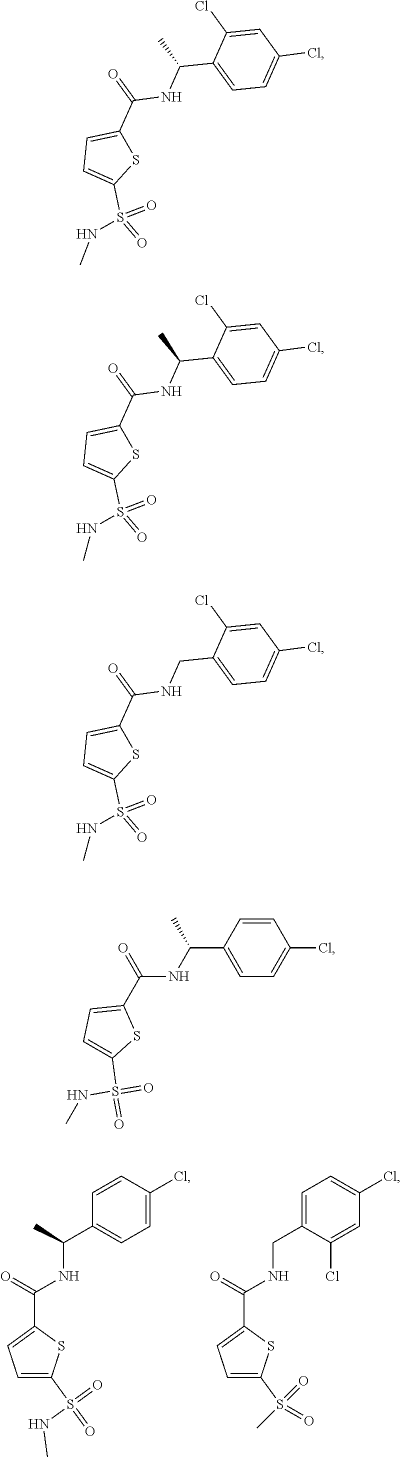 [SMILES: CNS(=O)(=O)c1ccc(C(=O)N[C@H](C)c2ccc(Cl)cc2Cl)s1 ; CNS(=O)(=O)c1ccc(C(=O)N[C@@H](C)c2ccc(Cl)cc2Cl)s1 ; CNS(=O)(=O)c1ccc(C(=O)NCc2ccc(Cl)cc2Cl)s1 ; CNS(=O)(=O)c1ccc(C(=O)N[C@H](C)c2ccc(Cl)cc2)s1 ; CNS(=O)(=O)c1ccc(C(=O)N[C@@H](C)c2ccc(Cl)cc2)s1 ; CS(=O)(=O)c1ccc(C(=O)NCc2ccc(Cl)cc2Cl)s1] 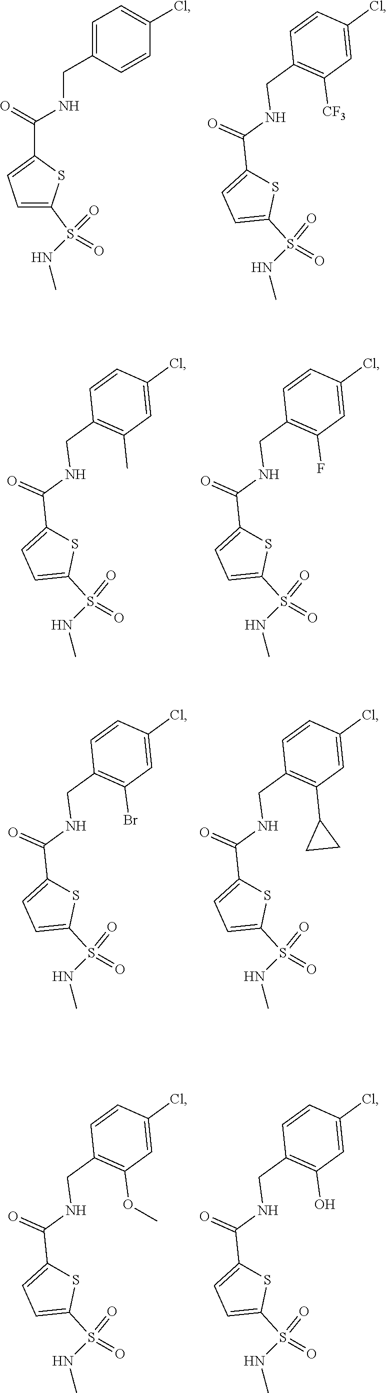 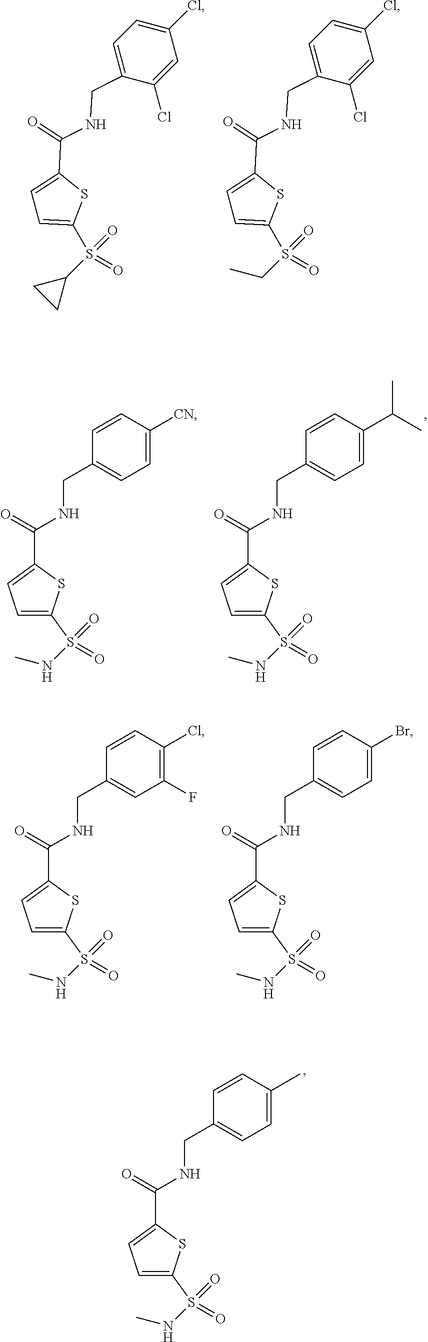 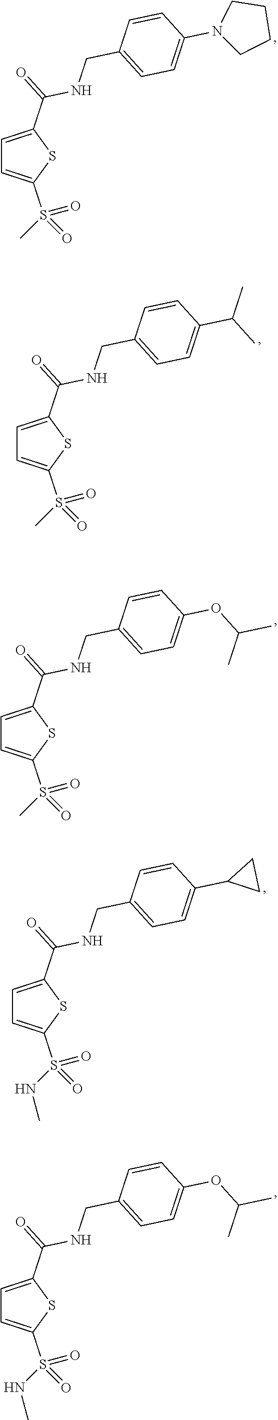 [SMILES: CS(=O)(=O)c1ccc(C(=O)NCc2ccc(N3CCCC3)cc2)s1 ; CC(C)c1ccc(CNC(=O)c2ccc(S(C)(=O)=O)s2)cc1 ; CC(C)Oc1ccc(CNC(=O)c2ccc(S(C)(=O)=O)s2)cc1 ; CNS(=O)(=O)c1ccc(C(=O)NCc2ccc(C3CC3)cc2)s1 ; CNS(=O)(=O)c1ccc(C(=O)NCc2ccc(OC(C)C)cc2)s1] 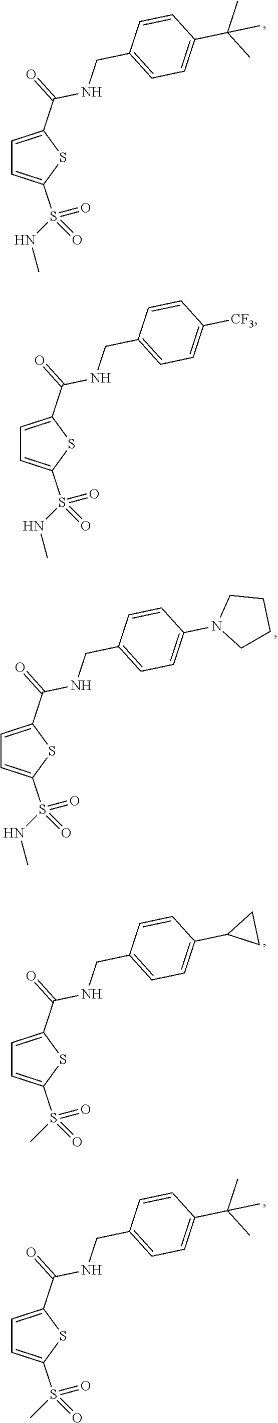 [SMILES: CNS(=O)(=O)c1ccc(C(=O)NCc2ccc(C(C)(C)C)cc2)s1 ; CNS(=O)(=O)c1ccc(C(=O)NCc2ccc(C(F)(F)F)cc2)s1 ; CNS(=O)(=O)c1ccc(C(=O)NCc2ccc(N3CCCC3)cc2)s1 ; CS(=O)(=O)c1ccc(C(=O)NCc2ccc(C3CC3)cc2)s1 ; CC(C)(C)c1ccc(CNC(=O)c2ccc(S(C)(=O)=O)s2)cc1] 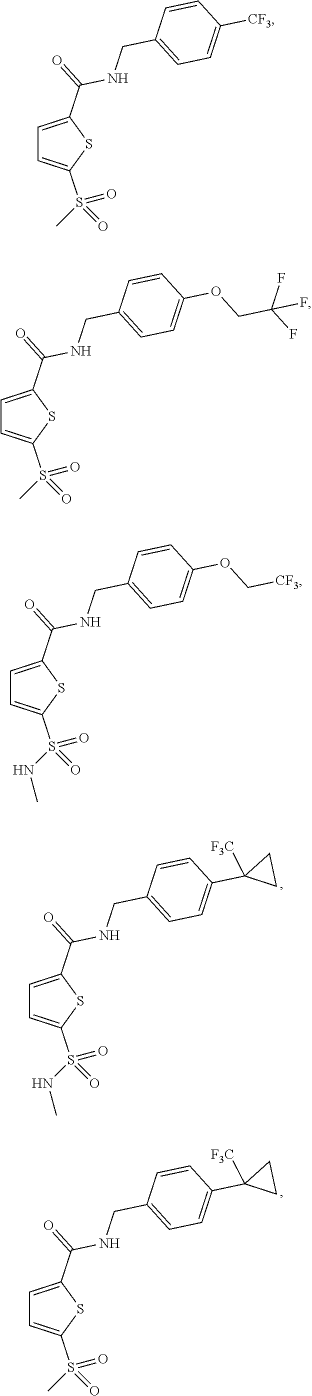 [SMILES: CS(=O)(=O)c1ccc(C(=O)NCc2ccc(C(F)(F)F)cc2)s1 ; CS(=O)(=O)c1ccc(C(=O)NCc2ccc(OCC(F)(F)F)cc2)s1 ; CNS(=O)(=O)c1ccc(C(=O)NCc2ccc(OCC(F)(F)F)cc2)s1 ; CNS(=O)(=O)c1ccc(C(=O)NCc2ccc(C3(C(F)(F)F)CC3)cc2)s1 ; CS(=O)(=O)c1ccc(C(=O)NCc2ccc(C3(C(F)(F)F)CC3)cc2)s1] 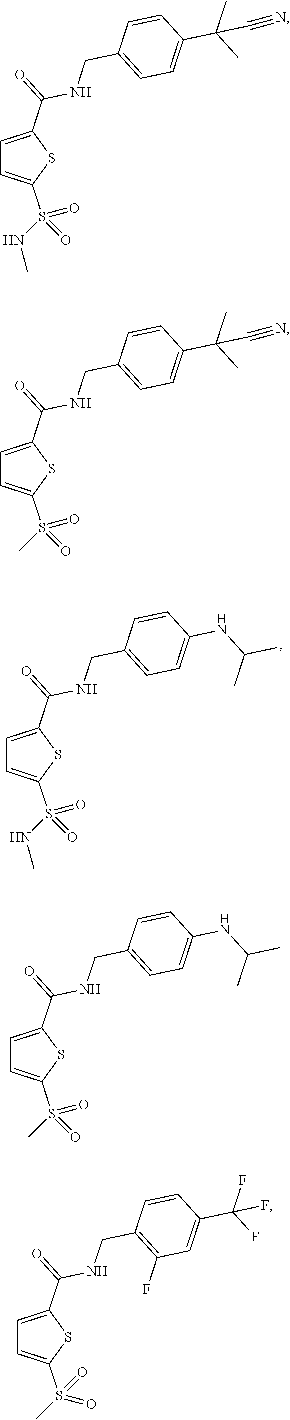 [SMILES: CNS(=O)(=O)c1ccc(C(=O)NCc2ccc(C(C)(C)C#N)cc2)s1 ; CC(C)(C#N)c1ccc(CNC(=O)c2ccc(S(C)(=O)=O)s2)cc1 ; CNS(=O)(=O)c1ccc(C(=O)NCc2ccc(NC(C)C)cc2)s1 ; CC(C)Nc1ccc(CNC(=O)c2ccc(S(C)(=O)=O)s2)cc1 ; CS(=O)(=O)c1ccc(C(=O)NCc2ccc(C(F)(F)F)cc2F)s1] 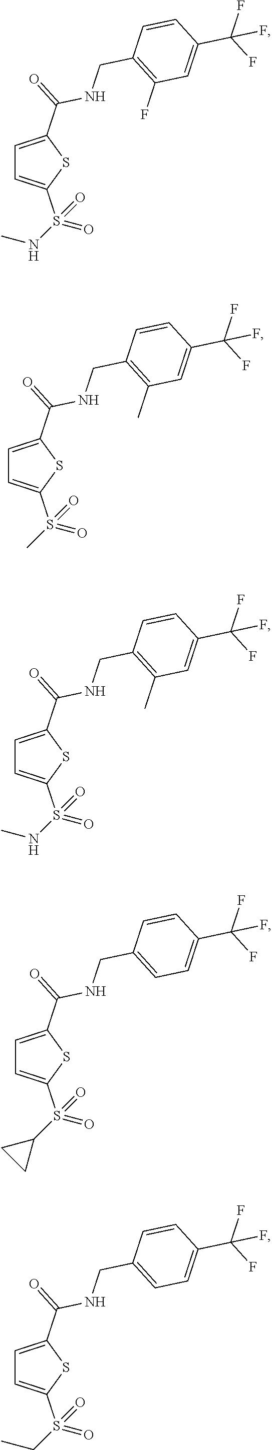 [SMILES: CNS(=O)(=O)c1ccc(C(=O)NCc2ccc(C(F)(F)F)cc2F)s1 ; Cc1cc(C(F)(F)F)ccc1CNC(=O)c1ccc(S(C)(=O)=O)s1 ; CNS(=O)(=O)c1ccc(C(=O)NCc2ccc(C(F)(F)F)cc2C)s1 ; O=C(NCc1ccc(C(F)(F)F)cc1)c1ccc(S(=O)(=O)C2CC2)s1 ; CCS(=O)(=O)c1ccc(C(=O)NCc2ccc(C(F)(F)F)cc2)s1] 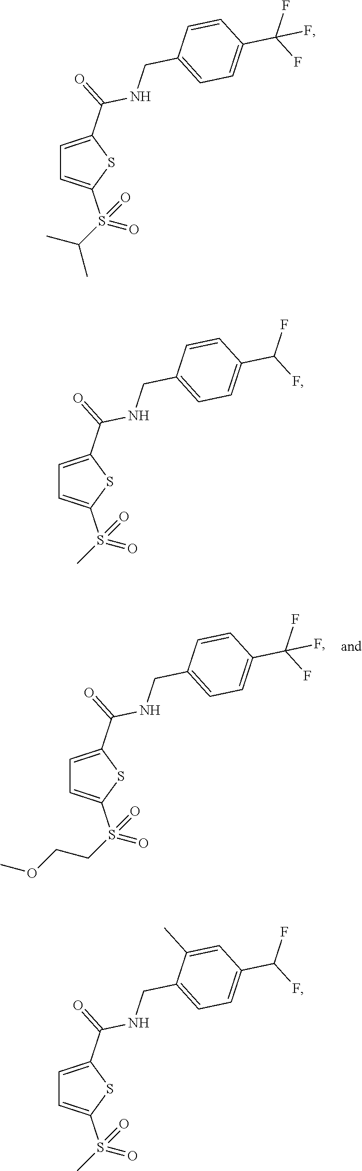 [SMILES: CC(C)S(=O)(=O)c1ccc(C(=O)NCc2ccc(C(F)(F)F)cc2)s1 ; CS(=O)(=O)c1ccc(C(=O)NCc2ccc(C(F)F)cc2)s1 ; COCCS(=O)(=O)c1ccc(C(=O)NCc2ccc(C(F)(F)F)cc2)s1 ; Cc1cc(C(F)F)ccc1CNC(=O)c1ccc(S(C)(=O)=O)s1]
or a pharmaceutically acceptable salt thereof.

43. A compound of Formula III: 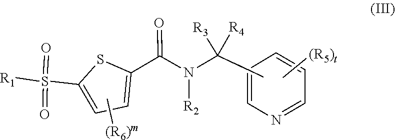
or a pharmaceutically acceptable salt thereof, wherein
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈ cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₂ is hydrogen;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
R₆ is C₁₋₆alkyl or C₁₋₆alkoxy;
t is 0, 1, 2, or 3; and
m is 0, 1, or 2.

44. The compound of claim 43, wherein R₁ is C₁₋₆alkyl.

45. The compound of claim 43 or 44, wherein R₁ is methyl.

46. The compound of any one of claims 43-45, wherein R₃ is hydrogen.

47. The compound of any one of claims 43-46, wherein R₄ is hydrogen.

48. The compound of any one of claims 43-47, wherein R₅ is each independently selected from the group consisting of halogen, cyano, —OH, C₁₋₆alkyl, C₁₋₆haloalkyl, C₁₋₆alkoxy, and C₃₋₈cycloalkyl, wherein the C₁₋₆alkyl or C₃₋₈cycloalkyl is optionally substituted with halogen, cyano, or C₁₋₆haloalkyl.

49. The compound of any one of claims 43-48, wherein R₅ is —CF₃.

50. The compound of any one of claims 43-49, wherein t is 1.

51. The compound of any one of claims 43-50, wherein m is 0.

52. The compound of claim 43, wherein the compound is selected from the group consisting of: 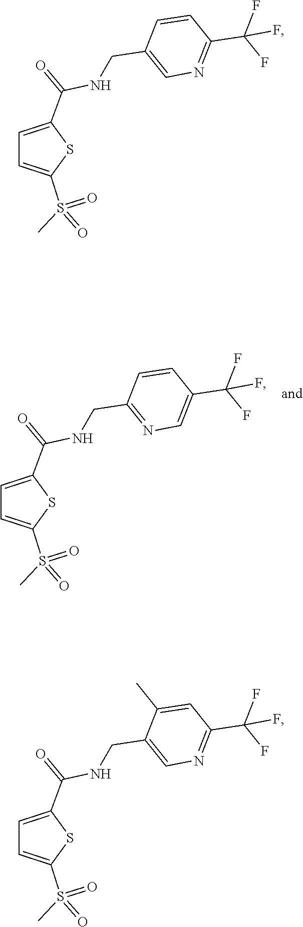 [SMILES: CS(=O)(=O)c1ccc(C(=O)NCc2ccc(C(F)(F)F)nc2)s1 ; CS(=O)(=O)c1ccc(C(=O)NCc2ccc(C(F)(F)F)cn2)s1 ; Cc1cc(C(F)(F)F)ncc1CNC(=O)c1ccc(S(C)(=O)=O)s1]
or a pharmaceutically acceptable salt thereof.

53. A compound selected from the group consisting of: 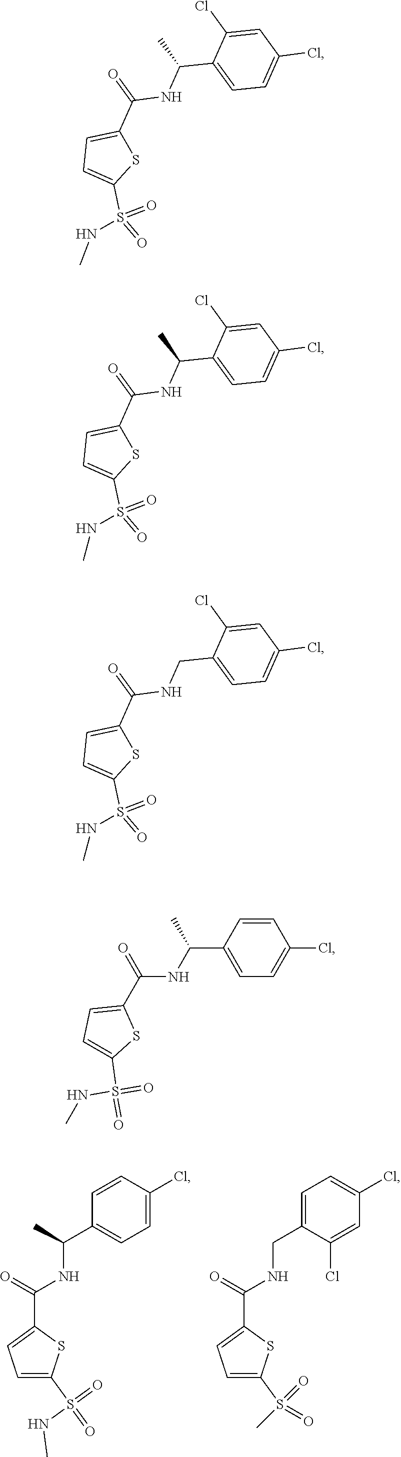 [SMILES: CNS(=O)(=O)c1ccc(C(=O)N[C@H](C)c2ccc(Cl)cc2Cl)s1 ; CNS(=O)(=O)c1ccc(C(=O)N[C@@H](C)c2ccc(Cl)cc2Cl)s1 ; CNS(=O)(=O)c1ccc(C(=O)NCc2ccc(Cl)cc2Cl)s1 ; CNS(=O)(=O)c1ccc(C(=O)N[C@H](C)c2ccc(Cl)cc2)s1 ; CNS(=O)(=O)c1ccc(C(=O)N[C@@H](C)c2ccc(Cl)cc2)s1 ; CS(=O)(=O)c1ccc(C(=O)NCc2ccc(Cl)cc2Cl)s1] 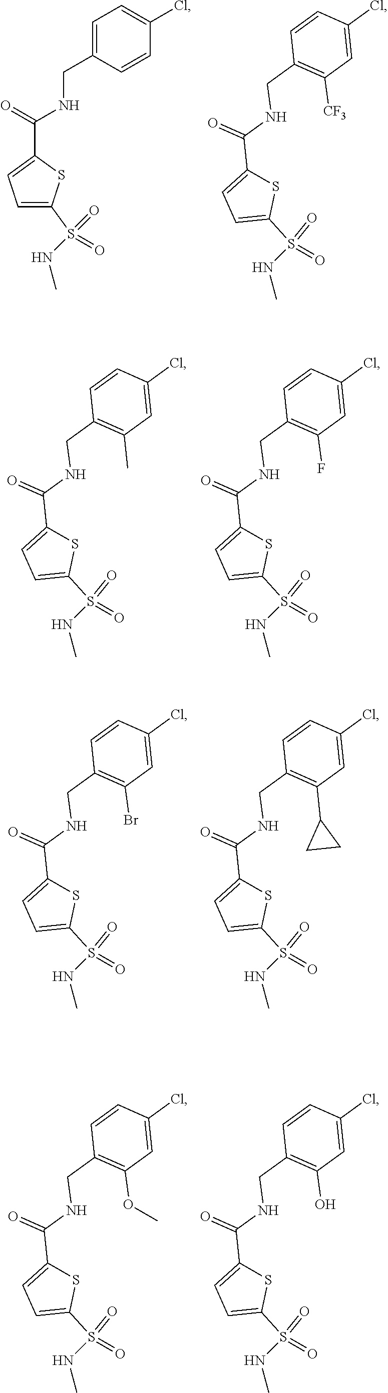 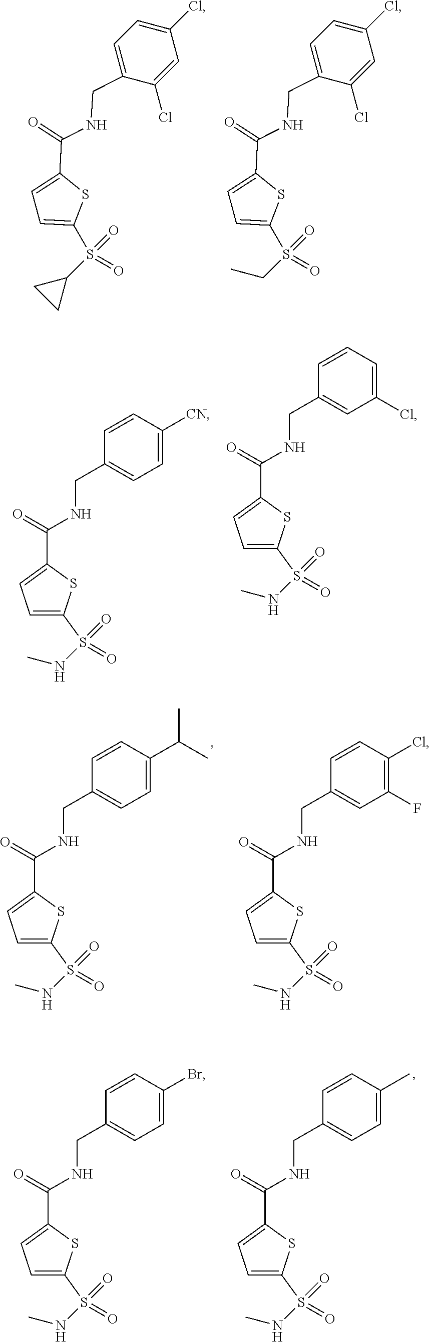 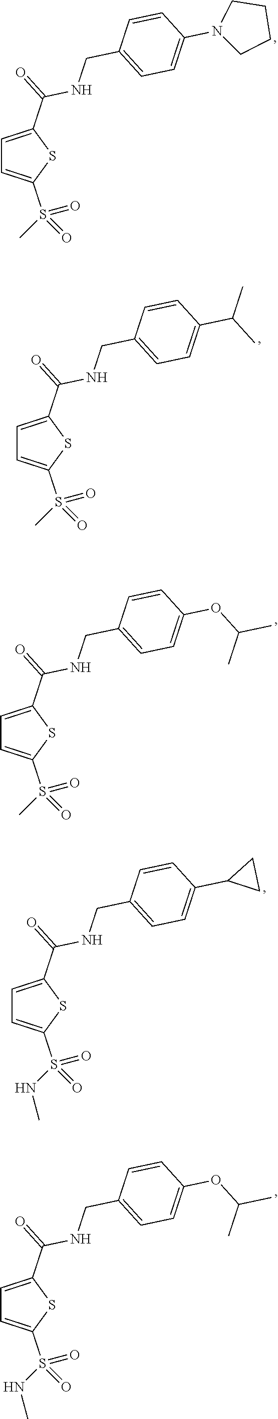 [SMILES: CS(=O)(=O)c1ccc(C(=O)NCc2ccc(N3CCCC3)cc2)s1 ; CC(C)c1ccc(CNC(=O)c2ccc(S(C)(=O)=O)s2)cc1 ; CC(C)Oc1ccc(CNC(=O)c2ccc(S(C)(=O)=O)s2)cc1 ; CNS(=O)(=O)c1ccc(C(=O)NCc2ccc(C3CC3)cc2)s1 ; CNS(=O)(=O)c1ccc(C(=O)NCc2ccc(OC(C)C)cc2)s1] 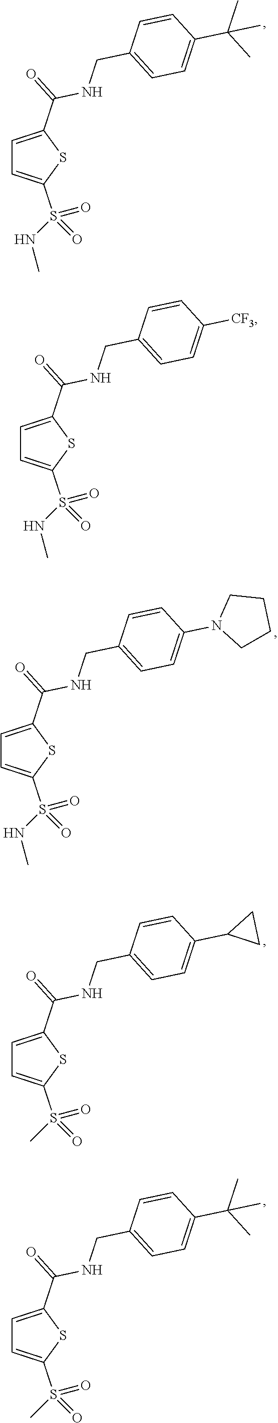 [SMILES: CNS(=O)(=O)c1ccc(C(=O)NCc2ccc(C(C)(C)C)cc2)s1 ; CNS(=O)(=O)c1ccc(C(=O)NCc2ccc(C(F)(F)F)cc2)s1 ; CNS(=O)(=O)c1ccc(C(=O)NCc2ccc(N3CCCC3)cc2)s1 ; CS(=O)(=O)c1ccc(C(=O)NCc2ccc(C3CC3)cc2)s1 ; CC(C)(C)c1ccc(CNC(=O)c2ccc(S(C)(=O)=O)s2)cc1] 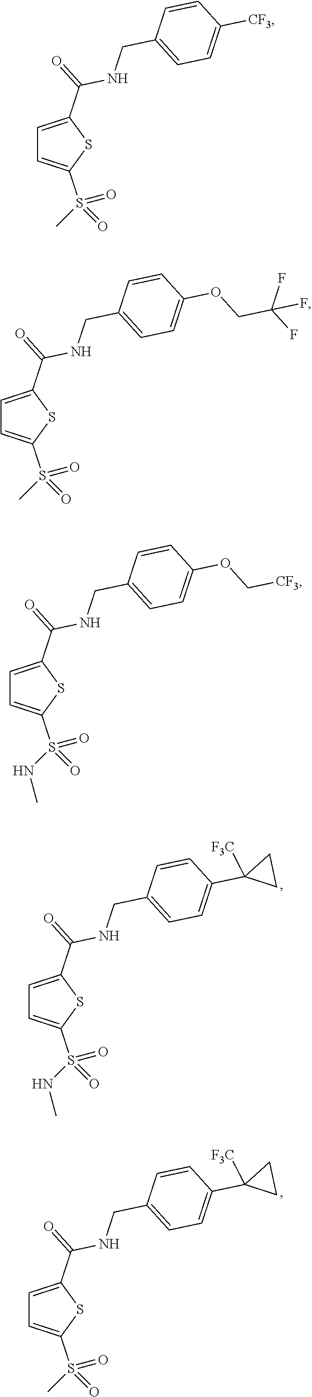 [SMILES: CS(=O)(=O)c1ccc(C(=O)NCc2ccc(C(F)(F)F)cc2)s1 ; CS(=O)(=O)c1ccc(C(=O)NCc2ccc(OCC(F)(F)F)cc2)s1 ; CNS(=O)(=O)c1ccc(C(=O)NCc2ccc(OCC(F)(F)F)cc2)s1 ; CNS(=O)(=O)c1ccc(C(=O)NCc2ccc(C3(C(F)(F)F)CC3)cc2)s1 ; CS(=O)(=O)c1ccc(C(=O)NCc2ccc(C3(C(F)(F)F)CC3)cc2)s1] 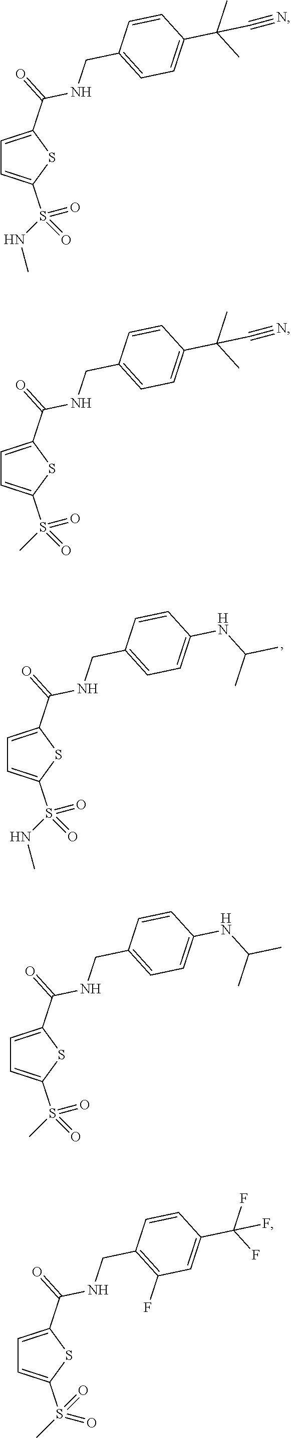 [SMILES: CNS(=O)(=O)c1ccc(C(=O)NCc2ccc(C(C)(C)C#N)cc2)s1 ; CC(C)(C#N)c1ccc(CNC(=O)c2ccc(S(C)(=O)=O)s2)cc1 ; CNS(=O)(=O)c1ccc(C(=O)NCc2ccc(NC(C)C)cc2)s1 ; CC(C)Nc1ccc(CNC(=O)c2ccc(S(C)(=O)=O)s2)cc1 ; CS(=O)(=O)c1ccc(C(=O)NCc2ccc(C(F)(F)F)cc2F)s1] 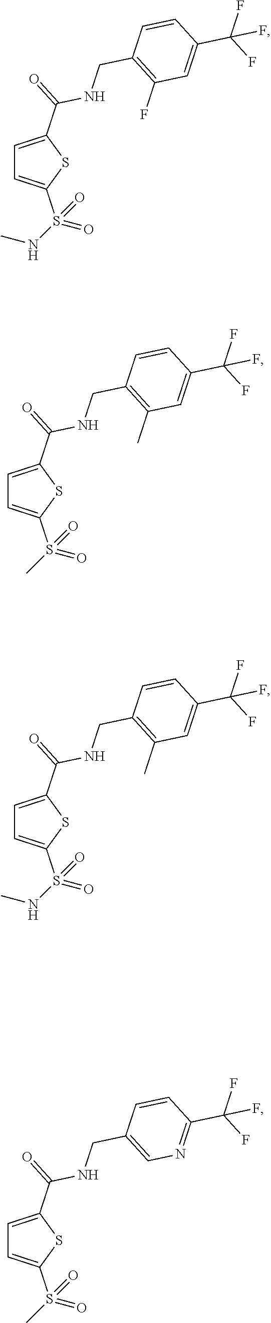 [SMILES: CNS(=O)(=O)c1ccc(C(=O)NCc2ccc(C(F)(F)F)cc2F)s1 ; Cc1cc(C(F)(F)F)ccc1CNC(=O)c1ccc(S(C)(=O)=O)s1 ; CNS(=O)(=O)c1ccc(C(=O)NCc2ccc(C(F)(F)F)cc2C)s1 ; CS(=O)(=O)c1ccc(C(=O)NCc2ccc(C(F)(F)F)nc2)s1] 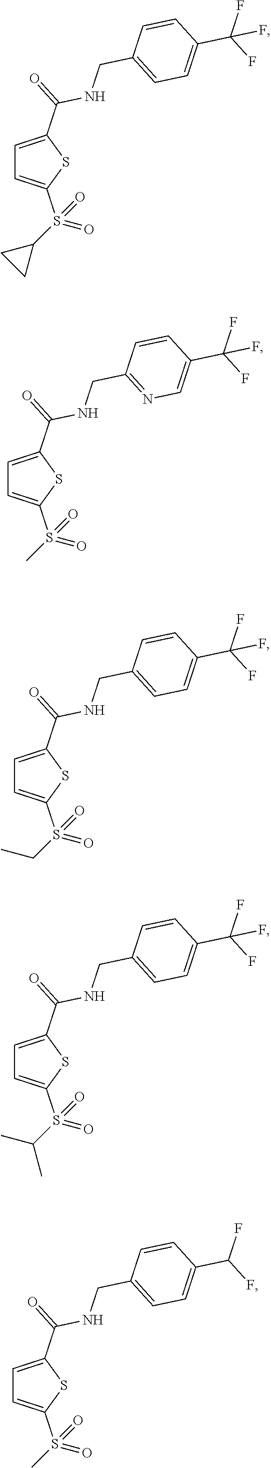 [SMILES: O=C(NCc1ccc(C(F)(F)F)cc1)c1ccc(S(=O)(=O)C2CC2)s1 ; CS(=O)(=O)c1ccc(C(=O)NCc2ccc(C(F)(F)F)cn2)s1 ; CCS(=O)(=O)c1ccc(C(=O)NCc2ccc(C(F)(F)F)cc2)s1 ; CC(C)S(=O)(=O)c1ccc(C(=O)NCc2ccc(C(F)(F)F)cc2)s1 ; CS(=O)(=O)c1ccc(C(=O)NCc2ccc(C(F)F)cc2)s1] 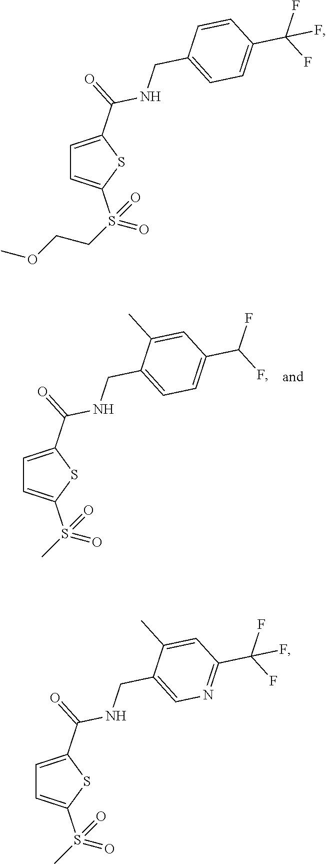 [SMILES: COCCS(=O)(=O)c1ccc(C(=O)NCc2ccc(C(F)(F)F)cc2)s1 ; Cc1cc(C(F)F)ccc1CNC(=O)c1ccc(S(C)(=O)=O)s1 ; Cc1cc(C(F)(F)F)ncc1CNC(=O)c1ccc(S(C)(=O)=O)s1]
or a pharmaceutically acceptable salt thereof.

54. A pharmaceutical composition comprising a compound of any one of claims 15-53 or a pharmaceutically acceptable salt thereof, and a pharmaceutically acceptable excipient.

55. A method of treating a neurological disease or disorder, wherein the method comprises administering to a subject in need thereof a compound of Formula A: 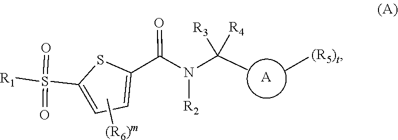
or a pharmaceutically acceptable salt thereof, wherein
A is phenyl or pyridyl;
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₂ is hydrogen or C₁₋₆alkyl;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
R₆ is C₁₋₆alkyl or C₁₋₆alkoxy;
t is 0, 1, 2, 3, or 4; and
m is 0, 1, or 2.

56. A method of treating a disease or condition associated with excessive neuronal excitability, wherein the method comprises administering to a subject in need thereof a compound of Formula A: 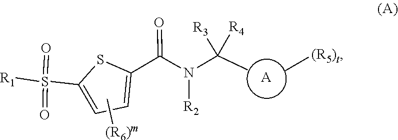
or a pharmaceutically acceptable salt thereof, wherein
A is phenyl or pyridyl;
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₂ is hydrogen or C₁₋₆alkyl;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
R₆ is C₁₋₆alkyl or C₁₋₆alkoxy;
t is 0, 1, 2, 3, or 4; and
m is 0, 1, or 2.

57. A method of treating a disease or condition associated with a gain-of-function mutation of a gene (e.g., KCNT1), wherein the method comprises administering to a subject in need thereof a compound of Formula A: 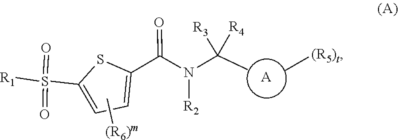
or a pharmaceutically acceptable salt thereof, wherein
A is phenyl or pyridyl;
R₁ is selected from the group consisting of C₁₋₆alkyl, C₃₋₈cycloalkyl, and —NHRₐ, wherein the C₁₋₆alkyl optionally substituted with C₁₋₆alkoxy;
Rₐ is selected from the group consisting of C₁₋₆alkyl, C₁₋₆alkylene-O—C₁₋₆alkyl, C₃₋₈cycloalkyl, or phenyl, wherein the C₃₋₈cycloalkyl or phenyl is optionally substituted with one or more halogen, C₁₋₆alkyl, C₁₋₆haloalkyl, or C₁₋₆alkoxy;
R₂ is hydrogen or C₁₋₆alkyl;
R₃ and R₄ are each independently selected from the group consisting of hydrogen, C₁₋₆alkyl, C₁₋₆alkylene-O—C₁₋₆alkyl, and C₁₋₆alkoxy;
R₅ is each independently selected from the group consisting of halogen, cyano, —OH, —NR_{c}R_{d}, C₁₋₆alkyl, C₁₋₆haloalkyl, C₁₋₆alkoxy, C₁₋₆haloalkoxy, C₁₋₆alkylene-O—C₁₋₆alkyl, C₃₋₈cycloalkyl, and 4-8 membered heterocyclyl, wherein the C₁₋₆alkyl, C₃₋₈cycloalkyl or 4-8 membered heterocyclyl is optionally substituted with one or more halogen, cyano, C₁₋₆alkyl, C₁₋₆haloalkyl, or C₁₋₆alkoxy;
R_{c} and R_{d} are each independently hydrogen or C₁₋₆alkyl;
R₆ is C₁₋₆alkyl or C₁₋₆alkoxy;
t is 0, 1, 2, 3, or 4; and
m is 0, 1, or 2.

58. A method of treating a neurological disease or disorder, wherein the method comprises administering to a subject in need thereof a compound of any one of claims 15-53 or a pharmaceutical composition of any one of claims 1-14 and 54.

59. A method of treating a disease or condition associated with excessive neuronal excitability, wherein the method comprises administering to a subject in need thereof a compound of any one of claims 15-53 or a pharmaceutical composition of any one of claims 1-14 and 54.

60. A method of treating a disease or condition associated with a gain-of-function mutation of a gene (e.g., KCNT1), wherein the method comprises administering to a subject in need thereof a compound of any one of claims 15-53 or a pharmaceutical composition of any one of claims 1-14 and 54.

61. The method of any one of claims 55-60, wherein the neurological disease or disorder, the disease or condition associated with excessive neuronal excitability, or the disease or condition associated with a gain-of-function mutation of a gene (e.g., KCNT1) is epilepsy, an epilepsy syndrome, or an encephalopathy.

62. The method of any one of claims 55-60, wherein the neurological disease or disorder, the disease or condition associated with excessive neuronal excitability, or the disease or condition associated with a gain-of-function mutation of a gene (e.g., KCNT1) is a genetic or pediatric epilepsy or a genetic or pediatric epilepsy syndrome.

63. The method of any one of claims 55-60, wherein the neurological disease or disorder, the disease or condition associated with excessive neuronal excitability, or the disease or condition associated with a gain-of-function mutation of a gene (e.g., KCNT1) is a cardiac dysfunction.

64. The method of any one of claims 55-60, wherein the neurological disease or disorder, the disease or condition associated with excessive neuronal excitability, or the disease or condition associated with a gain-of-function mutation of a gene (e.g., KCNT1) is selected from the group consisting of epilepsy and other encephalopathies (e.g., epilepsy of infancy with migrating focal seizures (MMFSI, EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox Gastaut syndrome, seizures (e.g., Generalized tonic clonic seizures, Asymmetric Tonic Seizures), leukodystrophy, leukoencephalopathy, intellectual disability, Multifocal Epilepsy, Drug resistant epilepsy, Temporal lobe epilepsy, or cerebellar ataxia).

65. The method of any one of claims 55-60, wherein the neurological disease or disorder, the disease or condition associated with excessive neuronal excitability, or the disease or condition associated with a gain-of-function mutation of a gene (e.g., KCNT1) is selected from the group consisting of cardiac arrhythmia, sudden unexpected death in epilepsy, Brugada syndrome, and myocardial infarction.

66. The method of any one of claims 55-60, wherein the neurological disease or disorder, the disease or condition associated with excessive neuronal excitability, or the disease or condition associated with a gain-of-function mutation of a gene (e.g., KCNT1) is selected from pain and related conditions (e.g. neuropathic pain, acute/chronic pain, migraine).

67. The method of any one of claims 55-60, the neurological disease or disorder, the disease or condition associated with excessive neuronal excitability, or the disease or condition associated with a gain-of-function mutation of a gene (e.g., KCNT1) is a muscle disorder (e.g. myotonia, neuromyotonia, cramp muscle spasms, spasticity).

68. The method of any one of claims 55-60, wherein the neurological disease or disorder, the disease or condition associated with excessive neuronal excitability, or the disease or condition associated with a gain-of-function mutation of a gene (e.g., KCNT1) is selected from itch and pruritis, ataxia and cerebellar ataxias.

69. The method of any one of claims 55-60, wherein the neurological disease or disorder, the disease or condition associated with excessive neuronal excitability, or the disease or condition associated with a gain-of-function mutation of a gene (e.g., KCNT1) is selected from psychiatric disorders (e.g. major depression, anxiety, bipolar disorder, schizophrenia).

70. The method of any one of claims 55-60, wherein the neurological disease or disorder or the disease or condition associated with excessive neuronal excitability and/or a gain-of-function mutation in a gene (e.g., KCNT1) is selected from the group consisting of learning disorders, Fragile X, neuronal plasticity, and autism spectrum disorders.

71. The method of any one of claims 55-60, wherein the neurological disease or disorder, the disease or condition associated with excessive neuronal excitability, or the disease or condition associated with a gain-of-function mutation of a gene (e.g., KCNT1) is selected from the group consisting of epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.
